SEQUENCE LISTING (223 sequences)

SEQ ID NO: 1 - protein, 1474 aa, synthetic construct
  REGION 1..1474: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 2 - DNA, 4425 bp, synthetic construct
  misc_feature 1..4425: note = -
  source 1..4425: mol_type = other DNA
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggaccccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggcattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaagctttcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtgaattcagcaggggtggctgaggtaggagtaacggtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagcacgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcggccgcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgcttga

SEQ ID NO: 3 - protein, 140 aa, synthetic construct
  REGION 1..140: note = -
EKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 4 - protein, 39 aa, synthetic construct
  REGION 1..39: note = -
PQLQQYEMHGPEGLRVGFYESDVMGRGHARLVHVEEPHT

SEQ ID NO: 5 - protein, 1636 aa, synthetic construct
  REGION 1..1636: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSGSGGSGGSGGSGGSGGSQVQLQESGGGLVQPGGSLRLSCAASGRTFSSYAMGWFRQAPGKEREFVAAIRWSGGYTYYTDSVKGRFTISRDNAKTTVYLQMNSLKPEDTAVYYCAATYLSSDYSRYALPQRPLDYDYWGQGTQVTVSSGSSGGSGGSGGSGGSNHVSRTEVSSNHVLIYLDKVSNQTLSLFFTVLQDVPVRDLKPAIVKVYDYYETDEFAIAEYNAPCSKDLGNA

SEQ ID NO: 6 - DNA, 4911 bp, synthetic construct
  misc_feature 1..4911: note = artificial
  source 1..4911: mol_type = other DNA
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggaccccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggcattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaagctttcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtgaattcagcaggggtggctgaggtaggagtaacggtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagcacgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcggccgcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctggaagcggcggaagcggcggaagcggcggaagcggcggaagcggcggatcccaggtgcagctgcaggagtctgggggaggattggtgcagccagggggctcgctgagactctcctgtgcagcctctggacgcaccttcagtagctatgccatgggctggttccgccaggctccagggaaggagcgtgagtttgtagcagctattaggtggagtggtggttacacatactatacagactccgtgaagggccgattcaccatctccagagacaacgccaagactacggtgtatctgcaaatgaacagcctgaaacctgaggacacggccgtttattactgtgcagcaacatacctctcctcggactatagccgctatgcgttgccccaaaggcccttggactatgactactggggccaggggacccaggtcaccgtctcctcaggctcgagcggcggaagcggcggaagcggcggaagcggcggaagcaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgcttga

SEQ ID NO: 7 - protein, 1745 aa, synthetic construct
  REGION 1..1745: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLAPTVAMLERSGSGGSGGSGGSGGSGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGSSGGSGGSGGSGGSNHVSRTEVSSNHVLIYLDKVSNQTLSLFFTVLQDVPVRDLKPAIVKVYDYYETDEFAIAEYNAPCSKDLGNA

SEQ ID NO: 8 - DNA, 5238 bp, synthetic construct
  misc_feature 1..5238: note = -
  source 1..5238: mol_type = other DNA
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggaccccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggcattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaagctttcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtgaattcagcaggggtggctgaggtaggagtaacggtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagcacgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcggccgcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctggccccaacagtggccatgcttgaaagatctggaagcggcggaagcggcggaagcggcggaagcggcggaagcggcggatccgaagtgcagctggttgaatctggcggaggactggttcagcctggcggatctctgagactgtcttgtgccgccagcggcttcaccttcagcgactcttggattcactgggtccgacaggcccctggcaaaggccttgaatgggttgcctggatcagcccttatggcggcagcacctactacgccgatagcgtgaagggcagattcaccatcagcgccgacaccagcaagaacaccgcctacctgcagatgaacagcctgagagccgaggacaccgccgtgtactattgtgccagaaggcattggcctggcggcttcgattattggggccagggaacactggtcaccgtttctggcggcggaggaagcggaggcggaggtagtggtggtggcggttctgacatccagatgacacagagccctagcagcctgtctgccagcgtgggagacagagtgaccatcacctgtagagccagccaggatgtgtctacagccgtggcctggtatcagcagaagcctggaaaggcccctaagctgctgatctacagcgccagctttctgtacagcggcgtgcccagcagattttctggcagcggctctggcaccgacttcaccctgaccatatctagcctgcagcctgaggacttcgccacctactattgccagcagtacctgtatcaccccgccacctttggccagggcaccaaggttgaaatcaaaggctcgagcggcggaagcggcggaagcggcggaagcggcggaagcaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgcttga

SEQ ID NO: 9 - protein, 1634 aa, synthetic construct
  REGION 1..1634: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLAPTVAMLERSGSGGSGGSGGSGGSGGSQVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVSSGSSGGSGGSGGSGGSNHVSRTEVSSNHVLIYLDKVSNQTLSLFFTVLQDVPVRDLKPAIVKVYDYYETDEFAIAEYNAPCSKDLGNA

SEQ ID NO: 10 - DNA, 4905 bp, synthetic construct
  misc_feature 1..4905: note = -
  source 1..4905: mol_type = other DNA
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggaccccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggcattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaagctttcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtgaattcagcaggggtggctgaggtaggagtaacggtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagcacgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcggccgcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctggccccaacagtggccatgcttgaaagatctggaagcggcggaagcggcggaagcggcggaagcggcggaagcggcggatcccaagtgcagctgcaggagagcggcggaggcctggtgcagccaggcggcagcctcagactgagctgcgccgcttctggaaagatgagctccaggcggtgcatggcctggttcagacaggcccctggcaaggaacgggaaagagtggccaagctgctgaccacaagcggatctacatatctggccgacagcgtgaagggcagattcaccatcagccagaacaatgccaaaagcacagtgtacctgcaaatgaacagcctgaagcctgaggacaccgccatgtactactgcgccgccgatagcttcgaggaccccacctgtaccctggtcacatctagcggcgcttttcagtactggggccagggcacccaggtgaccgtgtcctccggctcgagcggcggaagcggcggaagcggcggaagcggcggaagcaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgcttga

SEQ ID NO: 11 - protein, 1741 aa, synthetic construct
  REGION 1..1741: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSGSGGSGGSGGSGGSGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKGSSGGSGGSGGSGGSNHVSRTEVSSNHVLIYLDKVSNQTLSLFFTVLQDVPVRDLKPAIVKVYDYYETDEFAIAEYNAPCSKDLGNA

SEQ ID NO: 12 - DNA, 5226 bp, synthetic construct
  misc_feature 1..5226: note = -
  source 1..5226: mol_type = other DNA
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggaccccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggcattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaagctttcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtgaattcagcaggggtggctgaggtaggagtaacggtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagcacgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcggccgcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctggaagcggcggaagcggcggaagcggcggaagcggcggaagcggcggatcccaggtgcagctggtggagagcggcggcggcgtggtgcagcccggcaggagcctgaggctggactgcaaggccagcggcatcaccttcagcaacagcggcatgcactgggtgaggcaggcccccggcaagggcctggagtgggtggccgtgatctggtacgacggcagcaagaggtactacgccgacagcgtgaagggcaggttcaccatcagcagggacaacagcaagaacaccctgttcctgcagatgaacagcctgagggccgaggacaccgccgtgtactactgcgccaccaacgacgactactggggccagggcaccctggtgaccgtgagcagcggcggcggcggcagcggcggcggcggcagcggcggcggcggcagcgagatcgtgctgacccagagccccgccaccctgagcctgagccccggcgagagggccaccctgagctgcagggccagccagagcgtgagcagctacctggcctggtaccagcagaagcccggccaggcccccaggctgctgatctacgacgccagcaacagggccaccggcatccccgccaggttcagcggcagcggcagcggcaccgacttcaccctgaccatcagcagcctggagcccgaggacttcgccgtgtactactgccagcagagcagcaactggcccaggaccttcggccagggcaccaaggtggagatcaagggctcgagcggcggaagcggcggaagcggcggaagcggcggaagcaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgcttga

SEQ ID NO: 13 - protein, 1747 aa, synthetic construct
  REGION 1..1747: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSGSGGSGGSGGSGGSGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGGGSGGGGSGGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGSSGGSGGSGGSGGSNHVSRTEVSSNHVLIYLDKVSNQTLSLFFTVLQDVPVRDLKPAIVKVYDYYETDEFAIAEYNAPCSKDLGNA

SEQ ID NO: 14 - DNA, 5244 bp, synthetic construct
  misc_feature 1..5244: note = -
  source 1..5244: mol_type = other DNA
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggaccccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggcattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaagctttcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtgaattcagcaggggtggctgaggtaggagtaacggtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagcacgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcggccgcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctggaagcggcggaagcggcggaagcggcggaagcggcggaagcggcggatcccaggtgcagctggtggagagcggcggcggcgtggtgcagcccggcaggagcctgaggctgagctgcgccgccagcggcttcaccttcagcagctacaccatgcactgggtgaggcaggcccccggcaagggcctggagtgggtgaccttcatcagctacgacggcaacaacaagtactacgccgacagcgtgaagggcaggttcaccatcagcagggacaacagcaagaacaccctgtacctgcagatgaacagcctgagggccgaggacaccgccatctactactgcgccaggaccggctggctgggccccttcgactactggggccagggcaccctggtgaccgtgagcagcggcggcggcggcagcggcggcggcggcagcggcggcggcggcagcgagatcgtgctgacccagagccccggcaccctgagcctgagccccggcgagagggccaccctgagctgcagggccagccagagcgtgggcagcagctacctggcctggtaccagcagaagcccggccaggcccccaggctgctgatctacggcgccttcagcagggccaccggcatccccgacaggttcagcggcagcggcagcggcaccgacttcaccctgaccatcagcaggctggagcccgaggacttcgccgtgtactactgccagcagtacggcagcagcccctggaccttcggccagggcaccaaggtggagatcaagggctcgagcggcggaagcggcggaagcggcggaagcggcggaagcaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgcttga

SEQ ID NO: 15 - protein, 1747 aa, synthetic construct
  REGION 1..1747: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSGSGGSGGSGGSGGSGGSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGSSGGSGGSGGSGGSNHVSRTEVSSNHVLIYLDKVSNQTLSLFFTVLQDVPVRDLKPAIVKVYDYYETDEFAIAEYNAPCSKDLGNA

SEQ ID NO: 16 - DNA, 5244 bp, synthetic construct
  misc_feature 1..5244: note = -
  source 1..5244: mol_type = other DNA
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggaccccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggcattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaagctttcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtgaattcagcaggggtggctgaggtaggagtaacggtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagcacgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcggccgcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctggaagcggcggaagcggcggaagcggcggaagcggcggaagcggcggatcccaggtgcagctggtggagagcggcggcggcgtggtgcagcccggcaggagcctgaggctgagctgcgccgccagcggcttcaagttcagcggctacggcatgcactgggtgaggcaggcccccggcaagggcctggagtgggtggccgtgatctggtacgacggcagcaagaagtactacgtggacagcgtgaagggcaggttcaccatcagcagggacaacagcaagaacaccctgtacctgcagatgaacagcctgagggccgaggacaccgccgtgtactactgcgccaggcagatgggctactggcacttcgacctgtggggcaggggcaccctggtgaccgtgagcagcggcggcggcggcagcggcggcggcggcagcggcggcggcggcagcgagatcgtgctgacccagagccccgccaccctgagcctgagccccggcgagagggccaccctgagctgcagggccagccagagcgtgagcagctacctggcctggtaccagcagaagcccggccaggcccccaggctgctgatctacgacgccagcaacagggccaccggcatccccgccaggttcagcggcagcggcagcggcaccgacttcaccctgaccatcagcagcctggagcccgaggacttcgccgtgtactactgccagcagaggagcaactggccccccctgaccttcggcggcggcaccaaggtggagatcaagggctcgagcggcggaagcggcggaagcggcggaagcggcggaagcaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgcttga

SEQ ID NO: 17 - protein, 1749 aa, synthetic construct
  REGION 1..1749: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSGSGGSGGSGGSGGSGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGSSGGSGGSGGSGGSNHVSRTEVSSNHVLIYLDKVSNQTLSLFFTVLQDVPVRDLKPAIVKVYDYYETDEFAIAEYNAPCSKDLGNA

SEQ ID NO: 18 - DNA, 5250 bp, synthetic construct
  misc_feature 1..5250: note = -
  source 1..5250: mol_type = other DNA
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggaccccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggcattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaagctttcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtgaattcagcaggggtggctgaggtaggagtaacggtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagcacgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcggccgcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctggaagcggcggaagcggcggaagcggcggaagcggcggaagcggcggatccgacatcaagctgcagcagagcggcgccgagctggccaggcccggcgccagcgtgaagatgagctgcaagaccagcggctacaccttcaccaggtacaccatgcactgggtgaagcagaggcccggccagggcctggagtggatcggctacatcaaccccagcaggggctacaccaactacaaccagaagttcaaggacaaggccaccctgaccaccgacaagagcagcagcaccgcctacatgcagctgagcagcctgaccagcgaggacagcgccgtgtactactgcgccaggtactacgacgaccactactgcctggactactggggccagggcaccaccctgaccgtgagcagcgtggagggcggcagcggcggcagcggcggcagcggcggcagcggcggcgtggacgacatccagctgacccagagccccgccatcatgagcgccagccccggcgagaaggtgaccatgacctgcagggccagcagcagcgtgagctacatgaactggtaccagcagaagagcggcaccagccccaagaggtggatctacgacaccagcaaggtggccagcggcgtgccctacaggttcagcggcagcggcagcggcaccagctacagcctgaccatcagcagcatggaggccgaggacgccgccacctactactgccagcagtggagcagcaaccccctgaccttcggcgccggcaccaagctggagctgaagggctcgagcggcggaagcggcggaagcggcggaagcggcggaagcaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgcttga

SEQ ID NO: 19 - protein, 1752 aa, synthetic construct
  REGION 1..1752: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSGSGGSGGSGGSGGSGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFCGGTKVEIKRGSSGGSGGSGGSGGSNHVSRTEVSSNHVLIYLDKVSNQTLSLFFTVLQDVPVRDLKPAIVKVYDYYETDEFAIAEYNAPCSKDLGNA

SEQ ID NO: 20 - DNA, 5259 bp, synthetic construct
  misc_feature 1..5259: note = -
  source 1..5259: mol_type = other DNA
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggaccccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggcattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaagctttcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtgaattcagcaggggtggctgaggtaggagtaacggtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagcacgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcggccgcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctggaagcggcggaagcggcggaagcggcggaagcggcggaagcggcggatcccaggtgcagctgcagcagtggggcgccggcctgctgaagcccagcgagaccctgagcctgacctgcgccgtgtacggcggcagcttcagcggctactactggagctggatcaggcagagccccgagaagggcctggagtggatcggcgagatcaaccacggcggctacgtgacctacaaccccagcctggagagcagggtgaccatcagcgtggacaccagcaagaaccagttcagcctgaagctgagcagcgtgaccgccgccgacaccgccgtgtactactgcgccagggactacggccccggcaactacgactggtacttcgacctgtggggcaggggcaccctggtgaccgtgagcagcggcggcggcggcagcggcggcggcggcagcggcggcggcggcagcgagatcgtgctgacccagagccccgccaccctgagcctgagccccggcgagagggccaccctgagctgcagggccagccagagcgtgagcagctacctggcctggtaccagcagaagcccggccaggcccccaggctgctgatctacgacgccagcaacagggccaccggcatccccgccaggttcagcggcagcggcagcggcaccgacttcaccctgaccatcagcagcctggagcccgaggacttcgccgtgtactactgccagcagaggagcaactggccccccgccctgaccttctgcggcggcaccaaggtggagatcaagaggggctcgagcggcggaagcggcggaagcggcggaagcggcggaagcaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgcttga

SEQ ID NO: 21 - protein, 1749 aa, synthetic construct
  REGION 1..1749: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSGSGGSGGSGGSGGSGGSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGSSGGSGGSGGSGGSNHVSRTEVSSNHVLIYLDKVSNQTLSLFFTVLQDVPVRDLKPAIVKVYDYYETDEFAIAEYNAPCSKDLGNA

SEQ ID NO: 22 - DNA, 5250 bp, synthetic construct
  misc_feature 1..5250: note = -
  source 1..5250: mol_type = other DNA
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggaccccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggcattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaagctttcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtgaattcagcaggggtggctgaggtaggagtaacggtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagcacgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcggccgcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctggaagcggcggaagcggcggaagcggcggaagcggcggaagcggcggatccgaggtgcagctggtggagagcggcggcggcctggtgcagcccggcaggagcctgaggctgagctgcgccgccagcggcttcaccttcgacgactacgccatgcactgggtgaggcaggcccccggcaagggcctggagtgggtgagcgccatcacctggaacagcggccacatcgactacgccgacagcgtggagggcaggttcaccatcagcagggacaacgccaagaacagcctgtacctgcagatgaacagcctgagggccgaggacaccgccgtgtactactgcgccaaggtgagctacctgagcaccgccagcagcctggactactggggccagggcaccctggtgaccgtgagcagcggcggcggcggcagcggcggcggcggcagcggcggcggcggcagcgacatccagatgacccagagccccagcagcctgagcgccagcgtgggcgacagggtgaccatcacctgcagggccagccagggcatcaggaactacctggcctggtaccagcagaagcccggcaaggcccccaagctgctgatctacgccgccagcaccctgcagagcggcgtgcccagcaggttcagcggcagcggcagcggcaccgacttcaccctgaccatcagcagcctgcagcccgaggacgtggccacctactactgccagaggtacaacagggccccctacaccttcggccagggcaccaaggtggagatcaagggctcgagcggcggaagcggcggaagcggcggaagcggcggaagcaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgcttga

SEQ ID NO: 23 - protein, 1639 aa, synthetic construct
  REGION 1..1639: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSGSGGSGGSGGSGGSGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGSSGGSGGSGGSGGSNHVSRTEVSSNHVLIYLDKVSNQTLSLFFTVLQDVPVRDLKPAIVKVYDYYETDEFAIAEYNAPCSKDLGNA

SEQ ID NO: 24 - DNA, 4920 bp, synthetic construct
  misc_feature 1..4920: note = -
  source 1..4920: mol_type = other DNA
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggaccccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggcattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaagctttcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtgaattcagcaggggtggctgaggtaggagtaacggtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagcacgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcggccgcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctggaagcggcggaagcggcggaagcggcggaagcggcggaagcggcggatccgcccccaccagcagcagcaccaagaagacccagctgcagctggagcacctgctgctggacctgcagatgatcctgaacggcatcaacaactacaagaaccccaagctgaccaggatgctgaccttcaagttctacatgcccaagaaggccaccgagctgaagcacctgcagtgcctggaggaggagctgaagcccctggaggaggtgctgaacctggcccagagcaagaacttccacctgaggcccagggacctgatcagcaacatcaacgtgatcgtgctggagctgaagggcagcgagaccaccttcatgtgcgagtacgccgacgagaccgccaccatcgtggagttcctgaacaggtggatcaccttctgccagagcatcatcagcaccctgaccggctcgagcggcggaagcggcggaagcggcggaagcggcggaagcaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgcttga

SEQ ID NO: 25 - protein, 1631 aa, synthetic construct
  REGION 1..1631: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSGSGGSGGSGGSGGSGGSDSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEGSSGGSGGSGGSGGSNHVSRTEVSSNHVLIYLDKVSNQTLSLFFTVLQDVPVRDLKPAIVKVYDYYETDEFAIAEYNAPCSKDLGNA

SEQ ID NO: 26 - DNA, 4896 bp, synthetic construct
  misc_feature 1..4896: note = -
  source 1..4896: mol_type = other DNA
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggaccccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggcattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaagctttcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtgaattcagcaggggtggctgaggtaggagtaacggtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagcacgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcggccgcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctggaagcggcggaagcggcggaagcggcggaagcggcggaagcggcggatccgacagccccgacaggccctggaacccccccaccttcagccccgccctgctggtggtgaccgagggcgacaacgccaccttcacctgcagcttcagcaacaccagcgagagcttcgtgctgaactggtacaggatgagccccagcaaccagaccgacaagctggccgccttccccgaggacaggagccagcccggccaggacagcaggttcagggtgacccagctgcccaacggcagggacttccacatgagcgtggtgagggccaggaggaacgacagcggcacctacctgtgcggcgccatcagcctggcccccaaggcccagatcaaggagagcctgagggccgagctgagggtgaccgagaggagggccgagggctcgagcggcggaagcggcggaagcggcggaagcggcggaagcaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgcttga

SEQ ID NO: 27 - protein, 130 aa, synthetic construct
  REGION 1..130: note = -
QVQLQESGGGLVQPGGSLRLSCAASGRTFSSYAMGWFRQAPGKEREFVAAIRWSGGYTYYTDSVKGRFTISRDNAKTTVYLQMNSLKPEDTAVYYCAATYLSSDYSRYALPQRPLDYDYWGQGTQVTVSS

SEQ ID NO: 28 - protein, 239 aa, synthetic construct
  REGION 1..239: note = -
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 29 - protein, 128 aa, synthetic construct
  REGION 1..128: note = -
QVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVSS

SEQ ID NO: 30 - protein, 235 aa, synthetic construct
  REGION 1..235: note = -
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIK

SEQ ID NO: 31 - protein, 241 aa, synthetic construct
  REGION 1..241: note = -
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGGGSGGGGSGGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 32 - protein, 241 aa, synthetic construct
  REGION 1..241: note = -
QVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIK

SEQ ID NO: 33 - protein, 243 aa, synthetic construct
  REGION 1..243: note = -
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 34 - protein, 246 aa, synthetic construct
  REGION 1..246: note = -
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFCGGTKVEIKR

SEQ ID NO: 35 - protein, 243 aa, synthetic construct
  REGION 1..243: note = -
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIK

SEQ ID NO: 36 - protein, 133 aa, synthetic construct [sequence omitted]

SEQ ID NO: 37 - protein, 621 aa, synthetic construct [sequence omitted]

SEQ ID NO: 38 - protein, 221 aa, synthetic construct [sequence omitted]

SEQ ID NO: 39 - protein, 125 aa, synthetic construct
  REGION 1..125: note = -
DSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAE

SEQ ID NO: 40 - protein, 126 aa, synthetic construct [sequence omitted]

SEQ ID NO: 41 - protein, 203 aa, synthetic construct
  REGION 1..203: note = -
QSIKGNHLVKVYDYQEDGSVLLTCDAEAKNITWFKDGKMIGFLTEDKKKWNLGSNAKDPRGMYQCKGSQNKSKPLQVYYRMGGSGGSGGSGGSGGSGGSGGSGGSGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARV

SEQ ID NO: 42 - protein, 163 aa, synthetic construct
  REGION 1..163: note = -
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQ

SEQ ID NO: 43 - protein, 218 aa, synthetic construct
  REGION 1..218: note = -
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSALVTTTDFQIQTEMAATMETSIFTTEG

SEQ ID NO: 44 - protein, 231 aa, synthetic construct
  REGION 1..231: note = -
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 912 aa, synthetic construct
  REGION 1..912: note = -
MGKNKLLHPSLVLLLLVLLPTDASVSGWSHPQFEKGGGSGGGSGGSAWSHPQFEKGSLEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGSSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - DNA, 2739 bp, synthetic construct
  misc_feature 1..2739: note = -
  source 1..2739: mol_type = other DNA
atggggaagaacaaactccttcatccaagtctggttcttctcctcttggtcctcctgcccacagacgcctcagtctctggatggagccacccccagttcgagaagggcggcggcagcggcggcggcagcggcggcagcgcctggagccacccccagttcgagaagggatccctggaggagaagaaggtgtgccagggcaccagcaacaagctgacccagctgggcaccttcgaggaccacttcctgagcctgcagaggatgttcaacaactgcgaggtggtgctgggcaacctggagatcacctacgtgcagaggaactacgacctgagcttcctgaagaccatccaggaggtggccggctacgtgctgatcgccctgaacaccgtggagaggatccccctggagaacctgcagatcatcaggggcaacatgtactacgagaacagctacgccctggccgtgctgagcaactacgacgccaacaagaccggcctgaaggagctgcccatgaggaacctgcaggagatcctgcacggcgccgtgaggttcagcaacaaccccgccctgtgcaacgtggagagcatccagtggagggacatcgtgagcagcgacttcctgagcaacatgagcatggacttccagaaccacctgggcagctgccagaagtgcgaccccagctgccccaacggcagctgctggggcgccggcgaggagaactgccagaagctgaccaagatcatctgcgcccagcagtgcagcggcaggtgcaggggcaagagccccagcgactgctgccacaaccagtgcgccgccggctgcaccggccccagggagagcgactgcctggtgtgcaggaagttcagggacgaggccacctgcaaggacacctgcccccccctgatgctgtacaaccccaccacctaccagatggacgtgaaccccgagggcaagtacagcttcggcgccacctgcgtgaagaagtgccccaggaactacgtggtgaccgaccacggcagctgcgtgagggcctgcggcgccgacagctacgagatggaggaggacggcgtgaggaagtgcaagaagtgcgagggcccctgcaggaaggtgtgcaacggcatcggcatcggcgagttcaaggacagcctgagcatcaacgccaccaacatcaagcacttcaagaactgcaccagcatcagcggcgacctgcacatcctgcccgtggccttcaggggcgacagcttcacccacaccccccccctggacccccaggagctggacatcctgaagaccgtgaaggagatcaccggcttcctgctgatccaggcctggcccgagaacaggaccgacctgcacgccttcgagaacctggagatcatcaggggcaggaccaagcagcacggccagttcagcctggccgtggtgagcctgaacatcaccagcctgggcctgaggagcctgaaggagatcagcgacggcgacgtgatcatcagcggcaacaagaacctgtgctacgccaacaccatcaactggaagaagctgttcggcaccagcggccagaagaccaagatcatcagcaacaggggcgagaacagctgcaaggccaccggccaggtgtgccacgccctgtgcagccccgagggctgctggggccccgagcccagggactgcgtgagctgcaggaacgtgagcaggggcagggagtgcgtggacaagtgcaacctgctggagggcgagcccagggagttcgtggagaacagcgagtgcatccagtgccaccccgagtgcctgccccaggccatgaacatcacctgcaccggcaggggccccgacaactgcatccagtgcgcccactacatcgacggcccccactgcgtgaagacctgccccgccggcgtgatgggcgagaacaacaccctggtgtggaagtacgccgacgccggccacgtgtgccacctgtgccaccccaactgcacctacggctgcaccggccccggcctggagggctgccccaccaacggccccaagatccccagcggctcgagcccgaagagctgcgacaagacccacacatgtcctccttgccctgctcctgaactgctgggcggccctagcgtctttctgttcccccccaagcccaaggacaccctgatgatctctcggacccctgaggtgacctgcgtggtggtcgacgtgtcccacgaggaccccgaggtgaagttcaactggtacgtggacggcgtggaagtgcacaacgccaagaccaagcctagagaagagcagtacaactcaacatacagagtggtgtctgtgctgaccgtgctgcaccaggactggctgaacggaaaggaatacaagtgcaaggtgtccaacaaggccctgcccgcccctatcgagaagacaatcagcaaggccaagggccagcccagagagcctcaggtctacaccctgcctcctagcagagatgagctgaccaagaaccaggtgtccctgacctgtctggtgaaaggcttctaccccagcgacatcgccgtggaatgggagagcaacggccagcctgagaacaactacaagacgacccctccagtgctggacagcgatggcagcttcttcctgtacagcaagctgacagtggacaagtcccggtggcagcagggcaacgtgttcagctgcagcgtgatgcacgaggccctgcataatcactacacccagaagtctctgagcctcagccccggcaagtga

SEQ ID NO: 47 - protein, 512 aa, synthetic construct
  REGION 1..512: note = -
MGKNKLLHPSLVLLLLVLLPTDASVSGWSHPQFEKGGGSGGGSGGSAWSHPQFEKGSFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTGSSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - DNA, 1539 bp, synthetic construct
  misc_feature 1..1539: note = -
  source 1..1539: mol_type = other DNA
atggggaagaacaaactccttcatccaagtctggttcttctcctcttggtcctcctgcccacagacgcctcagtctctggatggagccacccccagttcgagaagggcggcggcagcggcggcggcagcggcggcagcgcctggagccacccccagttcgagaagggatcctttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggacccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactggctcgagcccgaagagctgcgacaagacccacacatgtcctccttgccctgctcctgaactgctgggcggccctagcgtctttctgttcccccccaagcccaaggacaccctgatgatctctcggacccctgaggtgacctgcgtggtggtcgacgtgtcccacgaggaccccgaggtgaagttcaactggtacgtggacggcgtggaagtgcacaacgccaagaccaagcctagagaagagcagtacaactcaacatacagagtggtgtctgtgctgaccgtgctgcaccaggactggctgaacggaaaggaatacaagtgcaaggtgtccaacaaggccctgcccgcccctatcgagaagacaatcagcaaggccaagggccagcccagagagcctcaggtctacaccctgcctcctagcagagatgagctgaccaagaaccaggtgtccctgacctgtctggtgaaaggcttctaccccagcgacatcgccgtggaatgggagagcaacggccagcctgagaacaactacaagacgacccctccagtgctggacagcgatggcagcttcttcctgtacagcaagctgacagtggacaagtcccggtggcagcagggcaacgtgttcagctgcagcgtgatgcacgaggccctgcataatcactacacccagaagtctctgagcctcagccccggcaagtga

SEQ ID NO: 49 - protein, 416 aa, synthetic construct
  REGION 1..416: note = -
MGKNKLLHPSLVLLLLVLLPTDASVSGWSHPQFEKGGGSGGGSGGSAWSHPQFEKGSDSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEGSSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - DNA, 1251 bp, synthetic construct
  misc_feature 1..1251: note = -
  source 1..1251: mol_type = other DNA
atggggaagaacaaactccttcatccaagtctggttcttctcctcttggtcctcctgcccacagacgcctcagtctctggatggagccacccccagttcgagaagggcggcggcagcggcggcggcagcggcggcagcgcctggagccacccccagttcgagaagggatccgacagccccgacaggccctggaacccccccaccttcagccccgccctgctggtggtgaccgagggcgacaacgccaccttcacctgcagcttcagcaacaccagcgagagcttcgtgctgaactggtacaggatgagccccagcaaccagaccgacaagctggccgccttccccgaggacaggagccagcccggccaggacagcaggttcagggtgacccagctgcccaacggcagggacttccacatgagcgtggtgagggccaggaggaacgacagcggcacctacctgtgcggcgccatcagcctggcccccaaggcccagatcaaggagagcctgagggccgagctgagggtgaccgagaggagggccgagggctcgagcccgaagagctgcgacaagacccacacatgtcctccttgccctgctcctgaactgctgggcggccctagcgtctttctgttcccccccaagcccaaggacaccctgatgatctctcggacccctgaggtgacctgcgtggtggtcgacgtgtcccacgaggaccccgaggtgaagttcaactggtacgtggacggcgtggaagtgcacaacgccaagaccaagcctagagaagagcagtacaactcaacatacagagtggtgtctgtgctgaccgtgctgcaccaggactggctgaacggaaaggaatacaagtgcaaggtgtccaacaaggccctgcccgcccctatcgagaagacaatcagcaaggccaagggccagcccagagagcctcaggtctacaccctgcctcctagcagagatgagctgaccaagaaccaggtgtccctgacctgtctggtgaaaggcttctaccccagcgacatcgccgtggaatgggagagcaacggccagcctgagaacaactacaagacgacccctccagtgctggacagcgatggcagcttcttcctgtacagcaagctgacagtggacaagtcccggtggcagcagggcaacgtgttcagctgcagcgtgatgcacgaggccctgcataatcactacacccagaagtctctgagcctcagccccggcaagtga

SEQ ID NO: 51 - protein, 417 aa, synthetic construct
  REGION 1..417: note = -
MGKNKLLHPSLVLLLLVLLPTDASVSGWSHPQFEKGGGSGGGSGGSAWSHPQFEKGSKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDGSSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 1254 aa, synthetic construct
  REGION 1..1254: note = -
ATGGGGAAGAACAAACTCCTTCATCCAAGTCTGGTTCTTCTCCTCTTGGTCCTCCTGCCCACAGACGCCTCAGTCTCTGGATGGAGCCACCCCCAGTTCGAGAAGGGCGGCGGCAGCGGCGGCGGCAGCGGCGGCAGCGCCTGGAGCCACCCCCAGTTCGAGAAGGGATCCAAGGCCATGCACGTGGCCCAGCCCGCCGTGGTGCTGGCCAGCAGCAGGGGCATCGCCAGCTTCGTGTGCGAGTACGCCAGCCCCGGCAAGGCCACCGAGGTGAGGGTGACCGTGCTGAGGCAGGCCGACAGCCAGGTGACCGAGGTGTGCGCCGCCACCTACATGATGGGCAACGAGCTGACCTTCCTGGACGACAGCATCTGCACCGGCACCAGCAGCGGCAACCAGGTGAACCTGACCATCCAGGGCCTGAGGGCCATGGACACCGGCCTGTACATCTGCAAGGTGGAGCTGATGTACCCCCCCCCCTACTACCTGGGCATCGGCAACGGCACCCAGATCTACGTGATCGACCCCGAGCCCTGCCCCGACAGCGACGGCTCGAGCCCGAAGAGCTGCGACAAGACCCACACATGTCCTCCTTGCCCTGCTCCTGAACTGCTGGGCGGCCCTAGCGTCTTTCTGTTCCCCCCCAAGCCCAAGGACACCCTGATGATCTCTCGGACCCCTGAGGTGACCTGCGTGGTGGTCGACGTGTCCCACGAGGACCCCGAGGTGAAGTTCAACTGGTACGTGGACGGCGTGGAAGTGCACAACGCCAAGACCAAGCCTAGAGAAGAGCAGTACAACTCAACATACAGAGTGGTGTCTGTGCTGACCGTGCTGCACCAGGACTGGCTGAACGGAAAGGAATACAAGTGCAAGGTGTCCAACAAGGCCCTGCCCGCCCCTATCGAGAAGACAATCAGCAAGGCCAAGGGCCAGCCCAGAGAGCCTCAGGTCTACACCCTGCCTCCTAGCAGAGATGAGCTGACCAAGAACCAGGTGTCCCTGACCTGTCTGGTGAAAGGCTTCTACCCCAGCGACATCGCCGTGGAATGGGAGAGCAACGGCCAGCCTGAGAACAACTACAAGACGACCCCTCCAGTGCTGGACAGCGATGGCAGCTTCTTCCTGTACAGCAAGCTGACAGTGGACAAGTCCCGGTGGCAGCAGGGCAACGTGTTCAGCTGCAGCGTGATGCACGAGGCCCTGCATAATCACTACACCCAGAAGTCTCTGAGCCTCAGCCCCGGCAAGTGA

SEQ ID NO: 53 - protein, 494 aa, synthetic construct
  REGION 1..494: note = -
MGKNKLLHPSLVLLLLVLLPTDASVSGWSHPQFEKGGGSGGGSGGSAWSHPQFEKGSQSIKGNHLVKVYDYQEDGSVLLTCDAEAKNITWFKDGKMIGFLTEDKKKWNLGSNAKDPRGMYQCKGSQNKSKPLQVYYRMGGSGGSGGSGGSGGSGGSGGSGGSGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVGSSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - DNA, 1485 bp, synthetic construct
  misc_feature 1..1485: note = -
  source 1..1485: mol_type = other DNA
atggggaagaacaaactccttcatccaagtctggttcttctcctcttggtcctcctgcccacagacgcctcagtctctggatggagccacccccagttcgagaagggcggcggcagcggcggcggcagcggcggcagcgcctggagccacccccagttcgagaagggatcccagagcatcaagggcaaccacctggtgaaggtgtacgactaccaggaggacggcagcgtgctgctgacctgcgacgccgaggccaagaacatcacctggttcaaggacggcaagatgatcggcttcctgaccgaggacaagaagaagtggaacctgggcagcaacgccaaggaccccaggggcatgtaccagtgcaagggcagccagaacaagagcaagcccctgcaggtgtactacaggatgggcggcagcggcggcagcggcggcagcggcggcagcggcggcagcggcggcagcggcggcagcggcggcagcggcagcgacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccaggggcagcaagcccgaggacgccaacttctacctgtacctgagggccagggtgggctcgagcccgaagagctgcgacaagacccacacatgtcctccttgccctgctcctgaactgctgggcggccctagcgtctttctgttcccccccaagcccaaggacaccctgatgatctctcggacccctgaggtgacctgcgtggtggtcgacgtgtcccacgaggaccccgaggtgaagttcaactggtacgtggacggcgtggaagtgcacaacgccaagaccaagcctagagaagagcagtacaactcaacatacagagtggtgtctgtgctgaccgtgctgcaccaggactggctgaacggaaaggaatacaagtgcaaggtgtccaacaaggccctgcccgcccctatcgagaagacaatcagcaaggccaagggccagcccagagagcctcaggtctacaccctgcctcctagcagagatgagctgaccaagaaccaggtgtccctgacctgtctggtgaaaggcttctaccccagcgacatcgccgtggaatgggagagcaacggccagcctgagaacaactacaagacgacccctccagtgctggacagcgatggcagcttcttcctgtacagcaagctgacagtggacaagtcccggtggcagcagggcaacgtgttcagctgcagcgtgatgcacgaggccctgcataatcactacacccagaagtctctgagcctcagccccggcaagtga

SEQ ID NO: 55 - protein, 454 aa, synthetic construct
  REGION 1..454: note = -
MGKNKLLHPSLVLLLLVLLPTDASVSGWSHPQFEKGGGSGGGSGGSAWSHPQFEKGSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQGSSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - DNA, 1365 bp, synthetic construct
  misc_feature 1..1365: note = -
  source 1..1365: mol_type = other DNA
atggggaagaacaaactccttcatccaagtctggttcttctcctcttggtcctcctgcccacagacgcctcagtctctggatggagccacccccagttcgagaagggcggcggcagcggcggcggcagcggcggcagcgcctggagccacccccagttcgagaagggatccctgcaggacccctgcagcaactgccccgccggcaccttctgcgacaacaacaggaaccagatctgcagcccctgcccccccaacagcttcagcagcgccggcggccagaggacctgcgacatctgcaggcagtgcaagggcgtgttcaggaccaggaaggagtgcagcagcaccagcaacgccgagtgcgactgcacccccggcttccactgcctgggcgccggctgcagcatgtgcgagcaggactgcaagcagggccaggagctgaccaagaagggctgcaaggactgctgcttcggcaccttcaacgaccagaagaggggcatctgcaggccctggaccaactgcagcctggacggcaagagcgtgctggtgaacggcaccaaggagagggacgtggtgtgcggccccagccccgccgacctgagccccggcgccagcagcgtgaccccccccgcccccgccagggagcccggccacagcccccagggctcgagcccgaagagctgcgacaagacccacacatgtcctccttgccctgctcctgaactgctgggcggccctagcgtctttctgttcccccccaagcccaaggacaccctgatgatctctcggacccctgaggtgacctgcgtggtggtcgacgtgtcccacgaggaccccgaggtgaagttcaactggtacgtggacggcgtggaagtgcacaacgccaagaccaagcctagagaagagcagtacaactcaacatacagagtggtgtctgtgctgaccgtgctgcaccaggactggctgaacggaaaggaatacaagtgcaaggtgtccaacaaggccctgcccgcccctatcgagaagacaatcagcaaggccaagggccagcccagagagcctcaggtctacaccctgcctcctagcagagatgagctgaccaagaaccaggtgtccctgacctgtctggtgaaaggcttctaccccagcgacatcgccgtggaatgggagagcaacggccagcctgagaacaactacaagacgacccctccagtgctggacagcgatggcagcttcttcctgtacagcaagctgacagtggacaagtcccggtggcagcagggcaacgtgttcagctgcagcgtgatgcacgaggccctgcataatcactacacccagaagtctctgagcctcagccccggcaagtga

SEQ ID NO: 57 - protein, 508 aa, synthetic construct
  REGION 1..508: note = -
MGKNKLLHPSLVLLLLVLLPTDASVSGWSHPQFEKGGGSGGGSGGSAWSHPQFEKGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSALVTTTDFQIQTEMAATMETSIFTTEGSSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - DNA, 1527 bp, synthetic construct
  misc_feature 1..1527: note = -
  source 1..1527: mol_type = other DNA
atggggaagaacaaactccttcatccaagtctggttcttctcctcttggtcctcctgcccacagacgcctcagtctctggatggagccacccccagttcgagaagggcggcggcagcggcggcggcagcggcggcagcgcctggagccacccccagttcgagaagggatccgagctgtgcgacgacgacccccccgagatcccccacgccaccttcaaggccatggcctacaaggagggcaccatgctgaactgcgagtgcaagaggggcttcaggaggatcaagagcggcagcctgtacatgctgtgcaccggcaacagcagccacagcagctgggacaaccagtgccagtgcaccagcagcgccaccaggaacaccaccaagcaggtgaccccccagcccgaggagcagaaggagaggaagaccaccgagatgcagagccccatgcagcccgtggaccaggccagcctgcccggccactgcagggagccccccccctgggagaacgaggccaccgagaggatctaccacttcgtggtgggccagatggtgtactaccagtgcgtgcagggctacagggccctgcacaggggccccgccgagagcgtgtgcaagatgacccacggcaagaccaggtggacccagccccagctgatctgcaccggcgagatggagaccagccagttccccggcgaggagaagccccaggccagccccgagggcaggcccgagagcgagaccagcgccctggtgaccaccaccgacttccagatccagaccgagatggccgccaccatggagaccagcatcttcaccaccgagggctcgagcccgaagagctgcgacaagacccacacatgtcctccttgccctgctcctgaactgctgggcggccctagcgtctttctgttcccccccaagcccaaggacaccctgatgatctctcggacccctgaggtgacctgcgtggtggtcgacgtgtcccacgaggaccccgaggtgaagttcaactggtacgtggacggcgtggaagtgcacaacgccaagaccaagcctagagaagagcagtacaactcaacatacagagtggtgtctgtgctgaccgtgctgcaccaggactggctgaacggaaaggaatacaagtgcaaggtgtccaacaaggccctgcccgcccctatcgagaagacaatcagcaaggccaagggccagcccagagagcctcaggtctacaccctgcctcctagcagagatgagctgaccaagaaccaggtgtccctgacctgtctggtgaaaggcttctaccccagcgacatcgccgtggaatgggagagcaacggccagcctgagaacaactacaagacgacccctccagtgctggacagcgatggcagcttcttcctgtacagcaagctgacagtggacaagtcccggtggcagcagggcaacgtgttcagctgcagcgtgatgcacgaggccctgcataatcactacacccagaagtctctgagcctcagccccggcaagtga

SEQ ID NO: 59 - protein, 214 aa, synthetic construct
  REGION 1..214: note = -
MGKNKLLHPSLVLLLLVLLPTDASVSGWSHPQFEKGGGSGGGSGGSAWSHPQFEKGSVRSSSRTPSDKPVAHVVANPQAEGQLQWLNDRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 60 - DNA, 648 bp, synthetic construct
  misc_feature 1..648: note = -
  source 1..648: mol_type = other DNA
atggggaagaacaaactccttcatccaagtctggttcttctcctcttggtcctcctgcccacagacgcctcagtctctggatggagccacccccagttcgagaagggcggcggcagcggcggcggcagcggcggcagcgcctggagccacccccagttcgagaagggatccgtgaggagcagcagcaggacccccagcgacaagcccgtggcccacgtggtggccaacccccaggccgagggccagctgcagtggctgaacgacagggccaacgccctgctggccaacggcgtggagctgagggacaaccagctggtggtgcccagcgagggcctgtacctgatctacagccaggtgctgttcaagggccagggctgccccagcacccacgtgctgctgacccacaccatcagcaggatcgccgtgagctaccagaccaaggtgaacctgctgagcgccatcaagagcccctgccagagggagacccccgagggcgccgaggccaagccctggtacgagcccatctacctgggcggcgtgttccagctggagaagggcgacaggctgagcgccgagatcaacaggcccgactacctggacttcgccgagagcggccaggtgtacttcggcatcatcgccctgtgatga

SEQ ID NO: 61 - protein, 688 aa, synthetic construct
  REGION 1..688: note = -
MGKNKLLHPSLVLLLLVLLPTDASVSGWSHPQFEKGGGSGGGSGGSAWSHPQFEKGSAPSPIIKFPGDVAPKTDKELAVQYLNTFYGCPKESCNLFVLKDTLKKMQKFFGLPQTGDLDQNTIETMRKPRCGNPDVANYNFFPRKPKWDKNQITYRIIGYTPDLDPETVDDAFARAFQVWSDVTPLRFSRIHDGEADIMINFGRWEHGDGYPFDGKDGLLAHAFAPGTGVGGDSHFDDDELWTLGEGQVVRVKYGNADGEYCKFPFLFNGKEYNSCTDTGRSDGFLWCSTTYNFEKDGKYGFCPHEALFTMGGNAEGQPCKFPFRFQGTSYDSCTTEGRTDGYRWCGTTEDYDRDKKYGFCPETAMSTVGGNSEGAPCVFPFTFLGNKYESCTSAGRSDGKMWCATTANYDDDRKWGFCPDQGYSLFLVAAHEFGHAMGLEHSQDPGALMAPIYTYTKNFRLSQDDIKGIQELYGASPDIDLGTGPTPTLGPVTPEICKQDIVFDGIAQIRGEIFFFKDRFIWRTVTPRDKPMGPLLVATFWPELPEKIDAVYEAPQEEKAVFFAGNEYWIYSASTLERGYPKPLTSLGLPPDVQRVDAAFNWSKNKKTYIFAGDKFWRYNEVKKKMDPGFPKLIADAWNAIPDNLDAVVDLQGGGHSYFFKGAYYLKLENQSLKSVKFGSIKSDWLGC

SEQ ID NO: 62 - DNA, 2067 bp, synthetic construct
  misc_feature 1..2067: note = -
  source 1..2067: mol_type = other DNA
atggggaagaacaaactccttcatccaagtctggttcttctcctcttggtcctcctgcccacagacgcctcagtctctggatggagccacccccagttcgagaagggcggcggcagcggcggcggcagcggcggcagcgcctggagccacccccagttcgagaagggatccgcgccgtcgcccatcatcaagttccccggcgatgtcgcccccaaaacggacaaagagttggcagtgcaatacctgaacaccttctatggctgccccaaggagagctgcaacctgtttgtgctgaaggacacactaaagaagatgcagaagttctttggactgccccagacaggtgatcttgaccagaataccatcgagaccatgcggaagccacgctgcggcaacccagatgtggccaactacaacttcttccctcgcaagcccaagtgggacaagaaccagatcacatacaggatcattggctacacacctgatctggacccagagacagtggatgatgcctttgctcgtgccttccaagtctggagcgatgtgaccccactgcggttttctcgaatccatgatggagaggcagacatcatgatcaactttggccgctgggagcatggcgatggatacccctttgacggtaaggacggactcctggctcatgccttcgccccaggcactggtgttgggggagactcccattttgatgacgatgagctatggaccttgggagaaggccaagtggtccgtgtgaagtatgggaacgccgatggggagtactgcaagttccccttcttgttcaatggcaaggagtacaacagctgcactgataccggccgcagcgatggcttcctctggtgctccaccacctacaactttgagaaggatggcaagtacggcttctgtccccatgaagccctgttcaccatgggcggcaacgctgaaggacagccctgcaagtttccattccgcttccagggcacatcctatgacagctgcaccactgagggccgcacggatggctaccgctggtgcggcaccactgaggactacgaccgcgacaagaagtatggcttctgccctgagaccgccatgtccactgttggtgggaactcagaaggtgccccctgtgtcttccccttcactttcctgggcaacaaatatgagagctgcaccagcgccggccgcagtgacggaaagatgtggtgtgcgaccacagccaactacgatgatgaccgcaagtggggcttctgccctgaccaagggtacagcctgttcctcgtggcagcccacgagtttggccacgccatggggctggagcactcccaagaccctggggccctgatggcacccatttacacctacaccaagaacttccgtctgtcccaggatgacatcaagggcattcaggagctctatggggcctctcctgacattgaccttggcaccggccccacccccacgctgggccctgtcactcctgagatctgcaaacaggacattgtatttgatggcatcgctcagatccgtggtgagatcttcttcttcaaggaccggttcatttggcggactgtgacgccacgtgacaagcccatggggcccctgctggtggccacattctggcctgagctcccggaaaagattgatgcggtatacgaggccccacaggaggagaaggctgtgttctttgcagggaatgaatactggatctactcagccagcaccctggagcgagggtaccccaagccactgaccagcctgggactgccccctgatgtccagcgagtggatgccgcctttaactggagcaaaaacaagaagacatacatctttgctggagacaaattctggagatacaatgaggtgaagaagaaaatggatcctggcttccccaagctcatcgcagatgcctggaatgccatccccgataacctggatgccgtcgtggacctgcagggcggcggtcacagctacttcttcaagggtgcctattacctgaagctggagaaccaaagtctgaagagcgtgaagtttggaagcatcaaatccgactggctaggctgctga

SEQ ID NO: 63 - protein, 1253 aa, synthetic construct
  REGION 1..1253: note = -
MGKNKLLHPSLVLLLLVLLPTDASVSGWSHPQFEKGGGSGGGSGGSAWSHPQFEKGSAIDAPKTCSPKQFACRDQITCISKGWRCDGERDCPDGSDEAPEICPQSKAQRCQPNEHNCLGTELCVPMSRLCNGVQDCMDGSDEGPHCRELQGNCSRLGCQHHCVPTLDGPTCYCNSSFQLQADGKTCKDFDECSVYGTCSQLCTNTDGSFICGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANAQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIVVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVGGAPPTVTLLRSERPPIFEIRMYDAQQQQVGTNKCRVNNGGCSSLCLATPGSRQCACAEDQVLDADGVTCLANPSYVPPPQCQPGEFACANSRCIQERWKCDGDNDCLDNSDEAPALCHQHTCPSDRFKCENNRCIPNRWLCDGDNDCGNSEDESNATCSARTCPPNQFSCASGRCIPISWTCDLDDDCGDRSDESASCAYPENLYFQGGSSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - DNA, 3762 bp, synthetic construct
  misc_feature 1..3762: note = -
  source 1..3762: mol_type = other DNA
atggggaagaacaaactccttcatccaagtctggttcttctcctcttggtcctcctgcccacagacgcctcagtctctggatggagccacccccagttcgagaagggcggcggcagcggcggcggcagcggcggcagcgcctggagccacccccagttcgagaagggatccgctatcgacgcccctaagacttgcagccccaagcagtttgcctgcagagatcaaataacctgtatctcaaagggctggcggtgcgacggtgagagggactgcccagacggatctgacgaggcccctgagatttgtccacagagtaaggcccagcgatgccagccaaacgagcataactgcctgggtactgagctgtgtgttcccatgtcccgcctctgcaatggggtccaggactgcatggacggctcagatgaggggccccactgccgagagctccaaggcaactgctctcgcctgggctgccagcaccattgtgtccccacactcgatgggcccacctgctactgcaacagcagctttcagcttcaggcagatggcaagacctgcaaagattttgatgagtgctcagtgtacggcacctgcagccagctatgcaccaacacagacggctccttcatatgtggctgtgttgaaggatacctcctgcagccggataaccgctcctgcaaggccaagaacgagccagtagaccggccccctgtgctgttgatagccaactcccagaacatcttggccacgtacctgagtggggcccaggtgtctaccatcacacctacgagcacgcggcagaccacagccatggacttcagctatgccaacgagaccgtatgctgggtgcatgttggggacagtgctgctcagacgcagctcaagtgtgcccgcatgcctggcctaaagggcttcgtggatgagcacaccatcaacatctccctcagtctgcaccacgtggaacagatggccatcgactggctgacaggcaacttctactttgtggatgacatcgatgataggatctttgtctgcaacagaaatggggacacatgtgtcacattgctagacctggaactctacaaccccaagggcattgccctggaccctgccatggggaaggtgtttttcactgactatgggcagatcccaaaggtggaacgctgtgacatggatgggcagaaccgcaccaagctcgtcgacagcaagattgtgtttcctcatggcatcacgctggacctggtcagccgccttgtctactgggcagatgcctatctggactatattgaagtggtggactatgagggcaagggccgccagaccatcatccagggcatcctgattgagcacctgtacggcctgactgtgtttgagaattatctctatgccaccaactcggacaatgccaatgcccagcagaagacgagtgtgatccgtgtgaaccgctttaacagcaccgagtaccaggttgtcacccgggtggacaagggtggtgccctccacatctaccaccagaggcgtcagccccgagtgaggagccatgcctgtgaaaacgaccagtatgggaagccgggtggctgctctgacatctgcctgctggccaacagccacaaggcgcggacctgccgctgccgttccggcttcagcctgggcagtgacgggaagtcatgcaagaagccggagcatgagctgttcctcgtgtatggcaagggccggccaggcatcatccggggcatggatatgggggccaaggtcccggatgagcacatgatccccattgaaaacctcatgaacccccgagccctggacttccacgctgagaccggcttcatctactttgccgacaccaccagctacctcattggccgccagaagattgatggcactgagcgggagaccatcctgaaggacggcatccacaatgtggagggtgtggccgtggactggatgggagacaatctgtactggacggacgatgggcccaaaaagacaatcagcgtggccaggctggagaaagctgctcagacccgcaagactttaatcgagggcaaaatgacacaccccagggctattgtggtggacccactcaatgggtggatgtactggacagactgggaggaggaccccaaggacagtcggcgtgggcggctggagagggcgtggatggatggctcacaccgagacatctttgtcacctccaagacagtgctttggcccaatgggctaagcctggacatcccggctgggcgcctctactgggtggatgccttctacgaccgcatcgagacgatactgctcaatggcacagaccggaagattgtgtatgaaggtcctgagctgaaccacgcctttggcctgtgtcaccatggcaactacctcttctggactgagtatcggagtggcagtgtctaccgcttggaacggggtgtaggaggcgcaccccccactgtgacccttctgcgcagtgagcggccccccatctttgagatccgaatgtatgatgcccagcagcagcaagttggcaccaacaaatgccgggtgaacaatggcggctgcagcagcctgtgcttggccacccctgggagccgccagtgcgcctgtgctgaggaccaggtgttggacgcagacggcgtcacttgcttggcgaacccatcctacgtgcctccaccccagtgccagccaggcgagtttgcctgtgccaacagccgctgcatccaggagcgctggaagtgtgacggagacaacgattgcctggacaacagtgatgaggccccagccctctgccatcagcacacctgcccctcggaccgattcaagtgcgagaacaaccggtgcatccccaaccgctggctctgcgacggggacaatgactgtgggaacagtgaagatgagtccaatgccacttgttcagcccgcacctgcccccccaaccagttctcctgtgccagtggccgctgcatccccatctcctggacgtgtgatctggatgacgactgtggggaccgctctgatgagtctgcttcgtgtgcctatcccgagaacctgtacttccagggcggctcgagcccgaagagctgcgacaagacccacacatgtcctccttgccctgctcctgaactgctgggcggccctagcgtctttctgttcccccccaagcccaaggacaccctgatgatctctcggacccctgaggtgacctgcgtggtggtcgacgtgtcccacgaggaccccgaggtgaagttcaactggtacgtggacggcgtggaagtgcacaacgccaagaccaagcctagagaagagcagtacaactcaacatacagagtggtgtctgtgctgaccgtgctgcaccaggactggctgaacggaaaggaatacaagtgcaaggtgtccaacaaggccctgcccgcccctatcgagaagacaatcagcaaggccaagggccagcccagagagcctcaggtctacaccctgcctcctagcagagatgagctgaccaagaaccaggtgtccctgacctgtctggtgaaaggcttctaccccagcgacatcgccgtggaatgggagagcaacggccagcctgagaacaactacaagacgacccctccagtgctggacagcgatggcagcttcttcctgtacagcaagctgacagtggacaagtcccggtggcagcagggcaacgtgttcagctgcagcgtgatgcacgaggccctgcataatcactacacccagaagtctctgagcctcagccccggcaagtga

SEQ ID NO: 65 - protein, 1474 aa, synthetic construct
  REGION 1..1474: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCGGSGGSGGSGGSGGSGGSGGSGGSGGSGGSGGSGGSGGSETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 66 - protein, 1474 aa, synthetic construct
  REGION 1..1474: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCGGSGGSGGSGGSGGKGGSGGSGGSGGSGGSGGSGGSGGSETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 67 - protein, 1474 aa, synthetic construct
  REGION 1..1474: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCGGSGGSGGSGGSGGSGHMHAALTAGGSGGSGGSGGSGGSETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 68 - protein, 1474 aa, synthetic construct
  REGION 1..1474: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCGGSGGSGGSGGSGGSGESLAYYTAGGSGGSGGSGGSGGSETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 69 - protein, 1474 aa, synthetic construct
  REGION 1..1474: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCGGSGGSGGSGGSGGSGRSLSRLTAGGSGGSGGSGGSGGSETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 70 - protein, 1474 aa, synthetic construct
  REGION 1..1474: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCGGSGGSGGSGGSGGGGSPQGIAGQGGSGGSGGSGGSGGSETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 71 - protein, 1467 aa, synthetic construct
  REGION 1..1467: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCGGSGGSGGSGGSGGGGSPQGIAGQGGSGGSGGETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 72 - protein, 1473 aa, synthetic construct
  REGION 1..1473: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCGGSGGSGGSGGSGGGGSPQGIAGQGGSGGLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 73 - protein, 1467 aa, synthetic construct
  REGION 1..1467: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCGGSGGSGGSGPQGIAGQSGGSGGSGGSGGSGGETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 74 - protein, 1738 aa, synthetic construct
  REGION 1..1738: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGICYTPVSSCNEKDMYSFLEDMGLKAFTNSKIRKPKMCGGSGGSGGSGPQGIAGQSGGSGGSGGSGGSGGETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLAPTVAMLERSGSGGSGGSGGSGGSGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGSSGGSGGSGGSGGSNHVSRTEVSSNHVLIYLDKVSNQTLSLFFTVLQDVPVRDLKPAIVKVYDYYETDEFAIAEYNAPCSKDLGNA

SEQ ID NO: 75 - DNA, 5217 bp, synthetic construct
  misc_feature 1..5217: note = -
  source 1..5217: mol_type = other DNA
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggaccccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggcattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatctgctatactccagtatcaagttgcaatgaaaaggatatgtacagcttcctagaggacatgggcttaaaagctttcaccaactcaaagattcgtaaacccaaaatgtgtggcggaagcggcggaagcggcggaagcggcccccagggcatcgccggccagagcggcggaagcggcggaagcggcggaagcggcggaagcggcggagagaccgtacgaaagtacttccctgagacatggatctgggatttggtggtggtgaattcagcaggggtggctgaggtaggagtaacggtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagcacgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcggccgcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctggccccaacagtggccatgcttgaaagatctggaagcggcggaagcggcggaagcggcggaagcggcggaagcggcggatccgaagtgcagctggttgaatctggcggaggactggttcagcctggcggatctctgagactgtcttgtgccgccagcggcttcaccttcagcgactcttggattcactgggtccgacaggcccctggcaaaggccttgaatgggttgcctggatcagcccttatggcggcagcacctactacgccgatagcgtgaagggcagattcaccatcagcgccgacaccagcaagaacaccgcctacctgcagatgaacagcctgagagccgaggacaccgccgtgtactattgtgccagaaggcattggcctggcggcttcgattattggggccagggaacactggtcaccgtttctggcggcggaggaagcggaggcggaggtagtggtggtggcggttctgacatccagatgacacagagccctagcagcctgtctgccagcgtgggagacagagtgaccatcacctgtagagccagccaggatgtgtctacagccgtggcctggtatcagcagaagcctggaaaggcccctaagctgctgatctacagcgccagctttctgtacagcggcgtgcccagcagattttctggcagcggctctggcaccgacttcaccctgaccatatctagcctgcagcctgaggacttcgccacctactattgccagcagtacctgtatcaccccgccacctttggccagggcaccaaggttgaaatcaaaggctcgagcggcggaagcggcggaagcggcggaagcggcggaagcaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgcttga

SEQ ID NO: 76 - protein, 1738 aa, synthetic construct
  REGION 1..1738: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGICYTPVSSCNEKDMYSFLEDMGLKAFTNSKIRKPKMCGGSGGSGGSGPSGIPGGSGGSGGSGGSGGSGGETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLAPTVAMLERSGSGGSGGSGGSGGSGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGSSGGSGGSGGSGGSNHVSRTEVSSNHVLIYLDKVSNQTLSLFFTVLQDVPVRDLKPAIVKVYDYYETDEFAIAEYNAPCSKDLGNA

SEQ ID NO: 77 - DNA, 5217 bp, synthetic construct
  misc_feature 1..5217: note = -
  source 1..5217: mol_type = other DNA
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggaccccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggcattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatctgctatactccagtatcaagttgcaatgaaaaggatatgtacagcttcctagaggacatgggcttaaaagctttcaccaactcaaagattcgtaaacccaaaatgtgtggcggaagcggcggaagcggcggaagcggccccagcggcatccccggcggaagcggcggaagcggcggaagcggcggaagcggcggaagcggcggagagaccgtacgaaagtacttccctgagacatggatctgggatttggtggtggtgaattcagcaggggtggctgaggtaggagtaacggtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagcacgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcggccgcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctggccccaacagtggccatgcttgaaagatctggaagcggcggaagcggcggaagcggcggaagcggcggaagcggcggatccgaagtgcagctggttgaatctggcggaggactggttcagcctggcggatctctgagactgtcttgtgccgccagcggcttcaccttcagcgactcttggattcactgggtccgacaggcccctggcaaaggccttgaatgggttgcctggatcagcccttatggcggcagcacctactacgccgatagcgtgaagggcagattcaccatcagcgccgacaccagcaagaacaccgcctacctgcagatgaacagcctgagagccgaggacaccgccgtgtactattgtgccagaaggcattggcctggcggcttcgattattggggccagggaacactggtcaccgtttctggcggcggaggaagcggaggcggaggtagtggtggtggcggttctgacatccagatgacacagagccctagcagcctgtctgccagcgtgggagacagagtgaccatcacctgtagagccagccaggatgtgtctacagccgtggcctggtatcagcagaagcctggaaaggcccctaagctgctgatctacagcgccagctttctgtacagcggcgtgcccagcagattttctggcagcggctctggcaccgacttcaccctgaccatatctagcctgcagcctgaggacttcgccacctactattgccagcagtacctgtatcaccccgccacctttggccagggcaccaaggttgaaatcaaaggctcgagcggcggaagcggcggaagcggcggaagcggcggaagcaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgcttga

SEQ ID NO: 78 - protein, 17 aa, synthetic construct
  REGION 1..17: note = -
GSGGSGGSGGSGGSGGS

SEQ ID NO: 79 - protein, 15 aa, synthetic construct
  REGION 1..15: note = -
GSSGGSGGSGGSGGS

SEQ ID NO: 80 - protein, 15 aa, synthetic construct
  REGION 1..15: note = -
GGSGGSGGSGGSGGS

SEQ ID NO: 81 - protein, 12 aa, synthetic construct
  REGION 1..12: note = -
GGSGGSGGSGGS

SEQ ID NO: 82 - protein, 18 aa, synthetic construct
  REGION 1..18: note = linker
GGSGGSGGSGGSGGSGGS

SEQ ID NO: 83 - protein, 15 aa, synthetic construct
  REGION 1..15: note = -
GSSGGSGGSGGSGGS

SEQ ID NO: 84 - protein, 1619 aa, synthetic construct
  REGION 1..1619: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPGGSGGSGGSGGSGGSQVQLQESGGGLVQPGGSLRLSCAASGRTFSSYAMGWFRQAPGKEREFVAAIRWSGGYTYYTDSVKGRFTISRDNAKTTVYLQMNSLKPEDTAVYYCAATYLSSDYSRYALPQRPLDYDYWGQGTQVTVSSGGSGGSGGSGGSHVSRTEVSSNHVLIYLDKVSNQTLSLFFTVLQDVPVRDLKPAIVKVYDYYETDEFAIAEYNAPCSKDLGNA

SEQ ID NO: 85 - DNA, 4860 bp, synthetic construct
  misc_feature 1..4860: note = -
  source 1..4860: mol_type = other DNA
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggatcccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggcattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaagctttcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtgaattcagcaggggtggctgaggtaggagtaacggtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagctcgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcggccgcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccagatatccctaagtgtcagttacacagggagccgctctgcctccaacatggcgatcgttgatgtgaagatggtctctggcttcattcccggcggaagcggcggaagcggcggaagcggcggaagcggcggaagccaggtgcagctgcaggagtctgggggaggattggtgcagccagggggctcgctgagactctcctgtgcagcctctggacgcaccttcagtagctatgccatgggctggttccgccaggctccagggaaggagcgtgagtttgtagcagctattaggtggagtggtggttacacatactatacagactccgtgaagggccgattcaccatctccagagacaacgccaagactacggtgtatctgcaaatgaacagcctgaaacctgaggacacggccgtttattactgtgcagcaacatacctctcctcggactatagccgctatgcgttgccccaaaggcccttggactatgactactggggccaggggacccaggtcaccgtctcctcaggcggaagcggcggaagcggcggaagcggcggaagccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgcttga

SEQ ID NO: 86 - protein, 1634 aa, synthetic construct
  REGION 1..1634: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLGGSGGSGGSGGSGGSGGSQVQLQESGGGLVQPGGSLRLSCAASGRTFSSYAMGWFRQAPGKEREFVAAIRWSGGYTYYTDSVKGRFTISRDNAKTTVYLQMNSLKPEDTAVYYCAATYLSSDYSRYALPQRPLDYDYWGQGTQVTVSSGSSGGSGGSGGSGGSVKMLERSNHVSRTEVSSNHVLIYLDKVSNQTLSLFFTVLQDVPVRDLKPAIVKVYDYYETDEFAIAEYNAPCSKDLGNA

SEQ ID NO: 87 - DNA, 4905 bp, synthetic construct
  misc_feature 1..4905: note = -
  source 1..4905: mol_type = other DNA
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggaccccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggcattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaagctttcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtgaattcagcaggggtggctgaggtaggagtaacggtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagcacgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcggccgcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgggcggaagcggcggaagcggcggaagcggcggaagcggcggaagcggcggatcccaggtgcagctgcaggagtctgggggaggattggtgcagccagggggctcgctgagactctcctgtgcagcctctggacgcaccttcagtagctatgccatgggctggttccgccaggctccagggaaggagcgtgagtttgtagcagctattaggtggagtggtggttacacatactatacagactccgtgaagggccgattcaccatctccagagacaacgccaagactacggtgtatctgcaaatgaacagcctgaaacctgaggacacggccgtttattactgtgcagcaacatacctctcctcggactatagccgctatgcgttgccccaaaggcccttggactatgactactggggccaggggacccaggtcaccgtctcctcaggctcgagcggcggaagcggcggaagcggcggaagcggcggaagcgtgaaaatgcttgaaagatctaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgcttga

SEQ ID NO: 88 - protein, 1740 aa, synthetic construct
  REGION 1..1740: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLGGSGGSGGSGGSGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGSSGGSGGSGGSGGSVKMLERSNHVSRTEVSSNHVLIYLDKVSNQTLSLFFTVLQDVPVRDLKPAIVKVYDYYETDEFAIAEYNAPCSKDLGNA

SEQ ID NO: 89 - DNA, 5223 bp, synthetic construct
  misc_feature 1..5223: note = -
  source 1..5223: mol_type = other DNA
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggaccccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggcattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaagctttcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtgaattcagcaggggtggctgaggtaggagtaacggtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagcacgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcggccgcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgggcggaagcggcggaagcggcggaagcggcggaagcggcggatccgaagtgcagctggttgaatctggcggaggactggttcagcctggcggatctctgagactgtcttgtgccgccagcggcttcaccttcagcgactcttggattcactgggtccgacaggcccctggcaaaggccttgaatgggttgcctggatcagcccttatggcggcagcacctactacgccgatagcgtgaagggcagattcaccatcagcgccgacaccagcaagaacaccgcctacctgcagatgaacagcctgagagccgaggacaccgccgtgtactattgtgccagaaggcattggcctggcggcttcgattattggggccagggaacactggtcaccgtttctggcggcggaggaagcggaggcggaggtagtggtggtggcggttctgacatccagatgacacagagccctagcagcctgtctgccagcgtgggagacagagtgaccatcacctgtagagccagccaggatgtgtctacagccgtggcctggtatcagcagaagcctggaaaggcccctaagctgctgatctacagcgccagctttctgtacagcggcgtgcccagcagattttctggcagcggctctggcaccgacttcaccctgaccatatctagcctgcagcctgaggacttcgccacctactattgccagcagtacctgtatcaccccgccacctttggccagggcaccaaggttgaaatcaaaggctcgagcggcggaagcggcggaagcggcggaagcggcggaagcgtgaaaatgcttgaaagatctaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgcttga

SEQ ID NO: 90 - protein, 1629 aa, synthetic construct
  REGION 1..1629: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLGGSGGSGGSGGSGGSQVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVSSGSSGGSGGSGGSGGSVKMLERSNHVSRTEVSSNHVLIYLDKVSNQTLSLFFTVLQDVPVRDLKPAIVKVYDYYETDEFAIAEYNAPCSKDLGNA

SEQ ID NO: 91 - DNA, 4890 bp, synthetic construct
  misc_feature 1..4890: note = -
  source 1..4890: mol_type = other DNA
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggaccccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggcattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaagctttcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtgaattcagcaggggtggctgaggtaggagtaacggtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagcacgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcggccgcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgggcggaagcggcggaagcggcggaagcggcggaagcggcggatcccaagtgcagctgcaggagagcggcggaggcctggtgcagccaggcggcagcctcagactgagctgcgccgcttctggaaagatgagctccaggcggtgcatggcctggttcagacaggcccctggcaaggaacgggaaagagtggccaagctgctgaccacaagcggatctacatatctggccgacagcgtgaagggcagattcaccatcagccagaacaatgccaaaagcacagtgtacctgcaaatgaacagcctgaagcctgaggacaccgccatgtactactgcgccgccgatagcttcgaggaccccacctgtaccctggtcacatctagcggcgcttttcagtactggggccagggcacccaggtgaccgtgtcctccggctcgagcggcggaagcggcggaagcggcggaagcggcggaagcgtgaaaatgcttgaaagatctaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgcttga

SEQ ID NO: 92 - protein, 1633 aa, synthetic construct
  REGION 1..1633: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVCKLKNSNHVSRTEVSSNHVLIYLDKVSNQTLSLFFTVLQDVPVRDLKPAIVKVYDYYETDEFAIAEYNAPCSKDLGNAGGSGGSGGSGGSGGSLEKEIAACEGSQVQLQESGGGLVQPGGSLRLSCAASGRTFSSYAMGWFRQAPGKEREFVAAIRWSGGYTYYTDSVKGRFTISRDNAKTTVYLQMNSLKPEDTAVYYCAATYLSSDYSRYALPQRPLDYDYWGQGTQVTVSSGSS

SEQ ID NO: 93 - DNA, 4902 bp, synthetic construct
  misc_feature 1..4902: note = -
  source 1..4902: mol_type = other DNA
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggaccccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggcattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaagctttcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtgaattcagcaggggtggctgaggtaggagtaacggtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagcacgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcggccgcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagcccaccgtgtgcaagctgaagaactctaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgctggcggaagcggcggaagcggcggaagcggcggaagcggcggaagcctggagaaggagatcgccgcctgcgagggatcccaggtgcagctgcaggagtctgggggaggattggtgcagccagggggctcgctgagactctcctgtgcagcctctggacgcaccttcagtagctatgccatgggctggttccgccaggctccagggaaggagcgtgagtttgtagcagctattaggtggagtggtggttacacatactatacagactccgtgaagggccgattcaccatctccagagacaacgccaagactacggtgtatctgcaaatgaacagcctgaaacctgaggacacggccgtttattactgtgcagcaacatacctctcctcggactatagccgctatgcgttgccccaaaggcccttggactatgactactggggccaggggacccaggtcaccgtctcctcaggctcgagctga

SEQ ID NO: 94 - protein, 1606 aa, synthetic construct
  REGION 1..1606: note = -
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNAGSQVQLQESGGGLVQPGGSLRLSCAASGRTFSSYAMGWFRQAPGKEREFVAAIRWSGGYTYYTDSVKGRFTISRDNAKTTVYLQMNSLKPEDTAVYYCAATYLSSDYSRYALPQRPLDYDYWGQGTQVTVSS

SEQ ID NO: 95 - DNA, 4821 bp, synthetic construct
  misc_feature 1..4821: note = -
  source 1..4821: mol_type = other DNA
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggaccccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggcattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaagctttcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtgaattcagcaggggtggctgaggtaggagtaacggtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagcacgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcggccgcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgctggaagccaggtgcagctgcaggagtctgggggaggattggtgcagccagggggctcgctgagactctcctgtgcagcctctggacgcaccttcagtagctatgccatgggctggttccgccaggctccagggaaggagcgtgagtttgtagcagctattaggtggagtggtggttacacatactatacagactccgtgaagggccgattcaccatctccagagacaacgccaagactacggtgtatctgcaaatgaacagcctgaaacctgaggacacggccgtttattactgtgcagcaacatacctctcctcggactatagccgctatgcgttgccccaaaggcccttggactatgactactggggccaggggacccaggtcaccgtctcctcatga

SEQ ID NO: 96 - protein, 8 aa, synthetic construct
  REGION 1..8: note = -
HMHAALTA

SEQ ID NO: 97 - protein, 8 aa, synthetic construct
  REGION 1..8: note = -
ESLAYYTA

SEQ ID NO: 98 - protein, 8 aa, synthetic construct
  REGION 1..8: note = -
RSLSRLTA

SEQ ID NO: 99 - protein, 7 aa, synthetic construct
  REGION 1..7: note = -
PQGIAGQ

SEQ ID NO: 100 - protein, 5 aa, synthetic construct
  REGION 1..5: note = -
PSGIP

SEQ ID NO: 101 - protein, 6 aa, synthetic construct
  REGION 1..6: note = -
GPEGLR

SEQ ID NO: 102 - protein, 8 aa, synthetic construct
  REGION 1..8: note = -
PRFKIIGG

SEQ ID NO: 103 - protein, 9 aa, synthetic construct
  REGION 1..9: note = -
SSRHRRALD

SEQ ID NO: 104 - protein, 14 aa, synthetic construct
  REGION 1..14: note = -
RKSSIIIRMRDVVL

SEQ ID NO: 105 - protein, 15 aa, synthetic construct
  REGION 1..15: note = -
SSSFDKGKYKKGDDA

SEQ ID NO: 106 - protein, 4 aa, synthetic construct
  REGION 1..4: note = -
IEGR

SEQ ID NO: 107 - protein, 4 aa, synthetic construct
  REGION 1..4: note = -
IDGR

SEQ ID NO: 108 - protein, 7 aa, synthetic construct
  REGION 1..7: note = -
GGSIDGR

SEQ ID NO: 109 - protein, 6 aa, synthetic construct
  REGION 1..6: note = -
PLGLWA

SEQ ID NO: 110 - protein, 8 aa, synthetic construct
  REGION 1..8: note = -
GPQGIAGQ

SEQ ID NO: 111 - protein, 8 aa, synthetic construct
  REGION 1..8: note = -
GPQGLLGA

SEQ ID NO: 112 - protein, 5 aa, synthetic construct
  REGION 1..5: note = -
GIAGQ

SEQ ID NO: 113 - protein, 8 aa, synthetic construct
  REGION 1..8: note = -
GPLGIAGI

SEQ ID NO: 114 - protein, 8 aa, synthetic construct
  REGION 1..8: note = -
YGAGLGVV

SEQ ID NO: 115 - protein, 8 aa, synthetic construct
  REGION 1..8: note = -
AGLGVVER

SEQ ID NO: 116 - protein, 8 aa, synthetic construct
  REGION 1..8: note = -
AGLGISST

SEQ ID NO: 117 - protein, 8 aa, synthetic construct
  REGION 1..8: note = -
DVAQFVLT

SEQ ID NO: 118 - protein, 8 aa, synthetic construct
  REGION 1..8: note = -
VAQFVLTE

SEQ ID NO: 119 - protein, 8 aa, synthetic construct
  REGION 1..8: note = -
AQFVLTEG

SEQ ID NO: 120 - protein, 8 aa, synthetic construct
  REGION 1..8: note = -
PVQPIGPQ

SEQ ID NO: 121 - protein, 13 aa, synthetic construct
  REGION 1..13: note = -
ISSGLLSGRSDNH

SEQ ID NO: 122 - protein, 6 aa, synthetic construct
  REGION 1..6: note = -
ISSGLL

SEQ ID NO: 123 - protein, 5 aa, synthetic construct
  REGION 1..5: note = -
RSDNH

SEQ ID NO: 124 - protein, 39 aa, synthetic construct
  REGION 1..39: note = Bait
GGSGGSGGSGGSGGSGGSGGSGGSGGSGGSGGSGGSGGS

SEQ ID NO: 125 - protein, 39 aa, synthetic construct
  REGION 1..39: note = Bait
GGSGGSGGSGGSGGKGGSGGSGGSGGSGGSGGSGGSGGS

SEQ ID NO: 126 - protein, 39 aa, synthetic construct
  REGION 1..39: note = Bait
GGSGGSGGSGGSGGSGHMHAALTAGGSGGSGGSGGSGGS

SEQ ID NO: 127 - protein, 39 aa, synthetic construct
  REGION 1..39: note = Bait
GGSGGSGGSGGSGGSGESLAYYTAGGSGGSGGSGGSGGS

SEQ ID NO: 128 - protein, 39 aa, synthetic construct
  REGION 1..39: note = BAIT
GGSGGSGGSGGSGGSGRSLSRLTAGGSGGSGGSGGSGGS

SEQ ID NO: 129 - protein, 39 aa, synthetic construct
  REGION 1..39: note = Bait
GGSGGSGGSGGSGGGGSPQGIAGQGGSGGSGGSGGSGGS

SEQ ID NO: 130 - protein, 39 aa, synthetic construct
  REGION 1..39: note = Bait
GGSGGSGGSGGSGGGGSPQGIAGQGGSGGSLVHVEEPHT

SEQ ID NO: 131 - protein, 32 aa, synthetic construct
  REGION 1..32: note = Bait
GGSGGSGGSGGSGGGGSPQGIAGQGGSGGSGG

SEQ ID NO: 132 - protein, 32 aa, synthetic construct
  REGION 1..32: note = Bait
GGSGGSGGSGPQGIAGQSGGSGGSGGSGGSGG

SEQ ID NO: 133 - protein, 32 aa, synthetic construct
  REGION 1..32: note = Bait
GGSGGSGGSGPSGIPGGSGGSGGSGGSGGSGG

SEQ ID NO: 134 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 135 - protein, 1744 aa, Homo sapiens
MRLLWGLIWASSFFTLSLQKPRLLLFSPSVVHLGVPLSVGVQLQDVPRGQVVKGSVFLRNPSRNNVPCSPKVDFTLSSERDFALLSLQVPLKDAKSCGLHQLLRGPEVQLVAHSPWLKDSLSRTTNIQGINLLFSSRRGHLFLQTDQPIYNPGQRVRYRVFALDQKMRPSTDTITVMVENSHGLRVRKKEVYMPSSIFQDDFVIPDISEPGTWKISARFSDGLESNSSTQFEVKKYVLPNFEVKITPGKPYILTVPGHLDEMQLDIQARYIYGKPVQGVAYVRFGLLDEDGKKTFFRGLESQTKLVNGQSHISLSKAEFQDALEKLNMGITDLQGLRLYVAAAIIESPGGEMEEAELTSWYFVSSPFSLDLSKTKRHLVPGAPFLLQALVREMSGSPASGIPVKVSATVSSPGSVPEVQDIQQNTDGSGQVSIPIIIPQTISELQLSVSAGSPHPAIARLTVAAPPSGGPGFLSIERPDSRPPRVGDTLNLNLRAVGSGATFSHYYYMILSRGQIVFMNREPKRTLTSVSVFVDHHLAPSFYFVAFYYHGDHPVANSLRVDVQAGACEGKLELSVDGAKQYRNGESVKLHLETDSLALVALGALDTALYAAGSKSHKPLNMGKVFEAMNSYDLGCGPGGGDSALQVFQAAGLAFSDGDQWTLSRKRLSCPKEKTTRKKRNVNFQKAINEKLGQYASPTAKRCCQDGVTRLPMMRSCEQRAARVQQPDCREPFLSCCQFAESLRKKSRDKGQAGLQRALEILQEEDLIDEDDIPVRSFFPENWLWRVETVDRFQILTLWLPDSLTTWEIHGLSLSKTKGLCVATPVQLRVFREFHLHLRLPMSVRRFEQLELRPVLYNYLDKNLTVSVHVSPVEGLCLAGGGGLAQQVLVPAGSARPVAFSVVPTAAAAVSLKVVARGSFEFPVGDAVSKVLQIEKEGAIHREELVYELNPLDHRGRTLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRDSSTWLTAFVLKVLSLAQEQVGGSPEKLQETSNWLLSQQQADGSFQDPCPVLDRSMQGGLVGNDETVALTAFVTIALHHGLAVFQDEGAEPLKQRVEASISKANSFLGEKASAGLLGAHAAAITAYALTLTKAPVDLLGVAHNNLMAMAQETGDNLYWGSVTGSQSNAVSPTPAPRNPSDPMPQAPALWIETTAYALLHLLLHEGKAEMADQASAWLTRQGSFQGGFRSTQDTVIALDALSAYWIASHTTEERGLNVTLSSTGRNGFKSHALQLNNRQIRGLEEELQFSLGSKINVKVGGNSKGTLKVLRTYNVLDMKNTTCQDLQIEVTVKGHVEYTMEANEDYEDYEYDELPAKDDPDAPLQPVTPLQLFEGRRNRRRREAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 136 - protein, 1744 aa, Homo sapiens
MRLLWGLIWASSFFTLSLQKPRLLLFSPSVVHLGVPLSVGVQLQDVPRGQVVKGSVFLRNPSRNNVPCSPKVDFTLSSERDFALLSLQVPLKDAKSCGLHQLLRGPEVQLVAHSPWLKDSLSRTTNIQGINLLFSSRRGHLFLQTDQPIYNPGQRVRYRVFALDQKMRPSTDTITVMVENSHGLRVRKKEVYMPSSIFQDDFVIPDISEPGTWKISARFSDGLESNSSTQFEVKKYVLPNFEVKITPGKPYILTVPGHLDEMQLDIQARYIYGKPVQGVAYVRFGLLDEDGKKTFFRGLESQTKLVNGQSHISLSKAEFQDALEKLNMGITDLQGLRLYVAAAIIESPGGEMEEAELTSWYFVSSPFSLDLSKTKRHLVPGAPFLLQALVREMSGSPASGIPVKVSATVSSPGSVPEVQDIQQNTDGSGQVSIPIIIPQTISELQLSVSAGSPHPAIARLTVAAPPSGGPGFLSIERPDSRPPRVGDTLNLNLRAVGSGATFSHYYYMILSRGQIVFMNREPKRTLTSVSVFVDHHLAPSFYFVAFYYHGDHPVANSLRVDVQAGACEGKLELSVDGAKQYRNGESVKLHLETDSLALVALGALDTALYAAGSKSHKPLNMGKVFEAMNSYDLGCGPGGGDSALQVFQAAGLAFSDGDQWTLSRKRLSCPKEKTTRKKRNVNFQKAINEKLGQYASPTAKRCCQDGVTRLPMMRSCEQRAARVQQPDCREPFLSCCQFAESLRKKSRDKGQAGLQRALEILQEEDLIDEDDIPVRSFFPENWLWRVETVDRFQILTLWLPDSLTTWEIHGLSLSKTKGLCVATPVQLRVFREFHLHLRLPMSVRRFEQLELRPVLYNYLDKNLTVSVHVSPVEGLCLAGGGGLAQQVLVPAGSARPVAFSVVPTAATAVSLKVVARGSFEFPVGDAVSKVLQIEKEGAIHREELVYELNPLDHRGRTLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRGSSTWLTAFVLKVLSLAQEQVGGSPEKLQETSNWLLSQQQADGSFQDLSPVIHRSMQGGLVGNDETVALTAFVTIALHHGLAVFQDEGAEPLKQRVEASISKASSFLGEKASAGLLGAHAAAITAYALTLTKAPADLRGVAHNNLMAMAQETGDNLYWGSVTGSQSNAVSPTPAPRNPSDPMPQAPALWIETTAYALLHLLLHEGKAEMADQAAAWLTRQGSFQGGFRSTQDTVIALDALSAYWIASHTTEERGLNVTLSSTGRNGFKSHALQLNNRQIRGLEEELQFSLGSKINVKVGGNSKGTLKVLRTYNVLDMKNTTCQDLQIEVTVKGHVEYTMEANEDYEDYEYDELPAKDDPDAPLQPVTPLQLFEGRRNRRRREAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 137 - protein, 1676 aa, Homo sapiens
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 138 - protein, 1474 aa, Homo sapiens
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDNEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 139 - protein, 1482 aa, Homo sapiens
MRKDRLLHLCLVLLLILLSASDSNSTEPQYMVLVPSLLHTEAPKKGCVLLSHLNETVTVSASLESGRENRSLFTDLVAEKDLFHCVSFTLPRISASSEVAFLSIQIKGPTQDFRKRNTVLVLNTQSLVFVQTDKPMYKPGQTVRFRVVSVDENFRPRNELIPLIYLENPRRNRIAQWQSLKLEAGINQLSFPLSSEPIQGSYRVVVQTESGGRIQHPFTVEEFVLPKFEVKVQVPKIISIMDEKVNITVCGEYTYGKPVPGLATVSLCRKLSRVLNCDKQEVCEEFSQQLNSNGCITQQVHTKMLQITNTGFEMKLRVEARIREEGTDLEVTANRISEITNIVSKLKFVKVDSHFRQGIPFFAQVLLVDGKGVPIPNKLFFISVNDANYYSNATTNEQGLAQFSINTTSISVNKLFVRVFTVHPNLCFHYSWVAEDHQGAQHTANRVFSLSGSYIHLEPVAGTLPCGHTETITAHYTLNRQAMGELSELSFHYLIMAKGVIVRSGTHTLPVESGDMKGSFALSFPVESDVAPIARMFIFAILPDGEVVGDSEKFEIENCLANKVDLSFSPAQSPPASHAHLQVAAAPQSLCALRAVDQSVLLMKPEAELSVSSVYNLLTVKDLTNFPDNVDQQEEEQGHCPRPFFIHNGAIYVPLSSNEADIYSFLKGMGLKVFTNSKIRKPKSCSVIPSVSAGAVGQGYYGAGLGVVERPYVPQLGTYNVIPLNNEQSSGPVPETVRSYFPETWIWELVAVNSSGVAEVGVTVPDTITEWKAGAFCLSEDAGLGISSTASLRAFQPFFVELTMPYSVIRGEVFTLKATVLNYLPKCIRVSVQLKASPAFLASQNTKGEESYCICGNERQTLSWTVTPKTLGNVNFSVSAEAMQSLELCGNEVVEVPEIKRKDTVIKTLLVEAEGIEQEKTFSSMTCASGANVSEQLSLKLPSNVVKESARASFSVLGDILGSAMQNIQNLLQMPYGCGEQNMVLFAPNIYVLNYLNETQQLTQEIKAKAVGYLITGYQRQLNYKHQDGSYSTFGERYGRNQGNTWLTAFVLKTFAQARSYIFIDEAHITQSLTWLSQMQKDNGCFRSSGSLLNNAIKGGVEDEATLSAYVTIALLEIPLPVTNPIVRNALFCLESAWNVAKEGTHGSHVYTKALLAYAFSLLGKQNQNREILNSLDKEAVKEDNLVHWERPQRPKAPVGHLYQTQAPSAEVEMTSYVLLAYLTAQPAPTSGDLTSATNIVKWIMKQQNAQGGFSSTQDTVVALHALSRYGAATFTRTEKTAQVTVQDSQTFSTNFQVDNNNLLLLQQISLPELPGEYVITVTGERCVYLQTSMKYNILPEKEDSPFALKVQTVPQTCDGHKAHTSFQISLTISYTGNRPASNMVIVDVKMVSGFIPLKPTVKMLERSSSVSRTEVSNNHVLIYVEQVTNQTLSFSFMVLQDIPVGDLKPAIVKVYDYYETDESVVAEYIAPCSTDTEHGNV

SEQ ID NO: 140 - protein, 1445 aa, Homo sapiens
MQGPPLLTAAHLLCVCTAALAVAPGPRFLVTAPGIIRPGGNVTIGVELLEHCPSQVTVKAELLKTASNLTVSVLEAEGVFEKGSFKTLTLPSLPLNSADEIYELRVTGRTQDEILFSNSTRLSFETKRISVFIQTDKALYKPKQEVKFRIVTLFSDFKPYKTSLNILIKDPKSNLIQQWLSQQSDLGVISKTFQLSSHPILGDWSIQVQVNDQTYYQSFQVSEYVLPKFEVTLQTPLYCSMNSKHLNGTITAKYTYGKPVKGDVTLTFLPLSFWGKKKNITKTFKINGSANFSFNDEEMKNVMDSSNGLSEYLDLSSPGPVEILTTVTESVTGISRNVSTNVFFKQHDYIIEFFDYTTVLKPSLNFTATVKVTRADGNQLTLEERRNNVVITVTQRNYTEYWSGSNSGNQKMEAVQKINYTVPQSGTFKIEFPILEDSSELQLKAYFLGSKSSMAVHSLFKSPSKTYIQLKTRDENIKVGSPFELVVSGNKRLKELSYMVVSRGQLVAVGKQNSTMFSLTPENSWTPKACVIVYYIEDDGEIISDVLKIPVQLVFKNKIKLYWSKVKAEPSEKVSLRISVTQPDSIVGIVAVDKSVNLMNASNDITMENVVHELELYNTGYYLGMFMNSFAVFQECGLWVLTDANLTKDYIDGVYDNAEYAERFMEENEGHIVDIHDFSLGSSPHVRKHFPETWIWLDTNMGYRIYQEFEVTVPDSITSWVATGFVISEDLGLGLTTTPVELQAFQPFFIFLNLPYSVIRGEEFALEITIFNYLKDATEVKVIIEKSDKFDILMTSNEINATGHQQTLLVPSEDGATVLFPIRPTHLGEIPITVTALSPTASDAVTQMILVKAEGIEKSYSQSILLDLTDNRLQSTLKTLSFSFPPNTVTGSERVQITAIGDVLGPSINGLASLIRMPYGCGEQNMINFAPNIYILDYLTKKKQLTDNLKEKALSFMRQGYQRELLYQREDGSFSAFGNYDPSGSTWLSAFVLRCFLEADPYIDIDQNVLHRTYTWLKGHQKSNGEFWDPGRVIHSELQGGNKSPVTLTAYIVTSLLGYRKYQPNIDVQESIHFLESEFSRGISDNYTLALITYALSSVGSPKAKEALNMLTWRAEQEGGMQFWVSSESKLSDSWQPRSLDIEVAAYALLSHFLQFQTSEGIPIMRWLSRQRNSLGGFASTQDTTVALKALSEFAALMNTERTNIQVTVTGPSSPSPVKFLIDTHNRLLLQTAELAVVQPTAVNISANGFGFAICQLNVVYNVKASGSSRRRRSIQNQEAFDLDVAVKENKDDLNHVDLNVCTSFSGPGRSGMALMEVNLLSGFMVPSEAISLSETVKKVEYDHGKLNLYLDSVNETQFCVNIPAVRNFKVSNTQDASVSIVDYYEPRRQAVRSYNSEVKLSSCDLCSDVQGCRPCEDGASGSHHHSSVIFIFCFKLLYFMELWL

SEQ ID NO: 141 - protein, 1885 aa, Homo sapiens
MSGALLWPLLPLLLLLLSARDGVRAAQPQAPGYLIAAPSVFRAGVEEVISVTIFNSPREVTVQAQLVAQGEPVVQSQGAILDKGTIKLKVPTGLRGQALLKVWGRGWQAEEGPLFHNQTSVTVDGRGASVFIQTDKPVYRPQHRVLISIFTVSPNLRPVNEKLEAYILDPRGSRMIEWRHLKPFCCGITNMSFPLSDQPVLGEWFIFVEMQGHAYNKSFEVQKYVLPKFELLIDPPRYIQDLDACETGTVRARYTFGKPVAGALMINMTVNGVGYYSHEVGRPVLRTTKILGSRDFDICVRDMIPADVPEHFRGRVSIWAMVTSVDGSQQVAFDDSTPVQRQLVDIRYSKDTRKQFKPGLAYVGKVELSYPDGSPAEGVTVQIKAELTPKDNIYTSEVVSQRGLVGFEIPSIPTSAQHVWLETKVMALNGKPVGAQYLPSYLSLGSWYSPSQCYLQLQPPSHPLQVGEEAYFSVKSTCPCNFTLYYEVAARGNIVLSGQQPAHTTQQRSKRAAPALEKPIRLTHLSETEPPPAPEAEVDVCVTSLHLAVTPSMVPLGRLLVFYVRENGEGVADSLQFAVETFFENQVSVTYSANETQPGEVVDLRIRAARGSCVCVAAVDKSVYLLRSGFRLTPAQVFQELEDYDVSDSFGVSREDGPFWWAGLTAQRRRRSSVFPWPWGITKDSGFAFTETGLVVMTDRVSLNHRQDGGLYTDEAVPAFQPHTGSLVAVAPSRHPPRTEKRKRTFFPETWIWHCLNISDPSGEGTLSVKVPDSITSWVGEAVALSTSQGLGIAEPSLLKTFKPFFVDFMLPALIIRGEQVKIPLSVYNYMGTCAEVYMKLSVPKGIQFVGHPGKRHVTKKMCVAPGEAEPIWVVLSFSDLGLNNITAKALAYGDTNCCRDGRSSKHPEENHADRRVPIGVDHVRRSVMVEAEGVPRAYTYSAFFCPSERVHISTPNKYEFQYVQRPLRLTRFDVAVRAHNDARVALSSGPQDTAGMIEIVLGGHQNTRSWISTSKMGEPVASAHTAKILSWDEFRTFWISWRGGLIQVGHGPEPSNESVIVAWTLPRPPEVQFIGFSTGWGSMGEFRIWRKMEVDESYSEAFTLGVPHGAIPGSERATASIIGDVMGPTLNHLNNLLRLPFGCGEQNMIHFAPNVFVLKYLQKTQQLSPEVERETTDYLVQGYQRQLTYKRQDGSYSAFGERDASGSMWLTAFVLKSFAQARSFIFVDPRELAAAKSWIIQQQQADGSFLAVGRVLNKDIQGGIHGTVPLTAYVVVALLETGTASEEERGSTDKARHFLESAAPLAMDPYSCALTTYALTLLRSPAAPEALRKLRSLAIMRDGVTHWSLSNSWDVDKGTFLSFSDRVSQSVVSAEVEMTAYALLTYTLLGDVAAALPVVKWLSQQRNALGGFSSTQDTCVALQALAEYAILSYAGGINLTVSLASTNLDYQETFELHRTNQKVLQTAAIPSLPTGLFVSAKGDGCCLMQIDVTYNVPDPVAKPAFQLLVSLQEPEAQGRPPPMPASAAEGSRGDWPPADDDDPAADQHHQEYKVMLEVCTRWLHAGSSNMAVLEVPLLSGFRADIESLEQLLLDKHMGMKRYEVAGRRVLFYFDEIPSRCLTCVRFRALRECVVGRTSALPVSVYDYYEPAFEATRFYNVSTHSPLARELCAGPACNEVERAPARGPGWFPGESGPAVAPEEGAAIARCGCDHDCGAQGNPVCGSDGVVYASACRLREAACRQAAPLEPAPPSCCALEQRLPASSSSTYGDDLASVAPGPLQQDVKLNGAGLEVEDSDPEPEGEAEDRVTAGPRPPVSSGNLESSTQSASPFHRWGQTPAPQRHSGRVVGAHRPGLLSPVFVYSPAFQSGGEEGLWMSNTCTLR

SEQ ID NO: 142 - protein, 1454 aa, Homo sapiens
MWAQLLLGMLALSPAIAEELPNYLVTLPARLNFPSVQKVCLDLSPGYSDVKFTVTLETKDKTQKLLEYSGLKKRHLHCISFLVPPPAGGTEEVATIRVSGVGNNISFEEKKKVLIQRQGNGTFVQTDKPLYTPGQQVYFRIVTMDSNFVPVNDKYSMVELQDPNSNRIAQWLEVVPEQGIVDLSFQLAPEAMLGTYTVAVAEGKTFGTFSVEEYVLPKFKVEVVEPKELSTVQESFLVKICCRYTYGKPMLGAVQVSVCQKANTYWYREVEREQLPDKCRNLSGQTDKTGCFSAPVDMATFDLIGYAYSHQINIVATVVEEGTGVEANATQNIYISPQMGSMTFEDTSNFYHPNFPFSGKIRVRGHDDSFLKNHLVFLVIYGTNGTFNQTLVTDNNGLAPFTLETSGWNGTDVSLEGKFQMEDLVYNPEQVPRYYQNAYLHLRPFYSTTRSFLGIHRLNGPLKCGQPQEVLVDYYIDPADASPDQEISFSYYLIGKGSLVMEGQKHLNSKKKGLKASFSLSLTFTSRLAPDPSLVIYAIFPSGGVVADKIQFSVEMCFDNQVSLGFSPSQQLPGAEVELQLQAAPGSLCALRAVDESVLLLRPDRELSNRSVYGMFPFWYGHYPYQVAEYDQCPVSGPWDFPQPLIDPMPQGHSSQRSIIWRPSFSEGTDLFSFFRDVGLKILSNAKIKKPVDCSHRSPEYSTAMGAGGGHPEAFESSTPLHQAEDSQVRQYFPETWLWDLFPIGNSGKEAVHVTVPDAITEWKAMSFCTSQSRGFGLSPTVGLTAFKPFFVDLTLPYSVVRGESFRLTATIFNYLKDCIRVQTDLAKSHEYQLESWADSQTSSCLCADDAKTHHWNITAVKLGHINFTISTKILDSNEPCGGQKGFVPQKGRSDTLIKPVLVKPEGVLVEKTHSSLLCPKGKVASESVSLELPVDIVPDSTKAYVTVLGDIMGTALQNLDGLVQMPSGCGEQNMVLFAPIIYVLQYLEKAGLLTEEIRSRAVGFLEIGYQKELMYKHSNGSYSAFGERDGNGNTWLTAFVTKCFGQAQKFIFIDPKNIQDALKWMAGNQLPSGCYANVGNLLHTAMKGGVDDEVSLTAYVTAALLEMGKDVDDPMVSQGLRCLKNSATSTTNLYTQALLAYIFSLAGEMDIRNILLKQLDQQAIISGESIYWSQKPTPSSNASPWSEPAAVDVELTAYALLAQLTKPSLTQKEIAKATSIVAWLAKQHNAYGGFSSTQDTVVALQALAKYATTAYMPSEEINLVVKSTENFQRTFNIQSVNRLVFQQDTLPNVPGMYTLEASGQGCVYVQTVLRYNILPPTNMKTFSLSVEIGKARCEQPTSPRSLTLTIHTSYVGSRSSSNMAIVEVKMLSGFSPMEGTNQLLLQQPLVKKVEFGTDTLNIYLDELIKNTQTYTFTISQSVLVTNLKPATIKVYDYYLPDEQATIQYSDPCE

SEQ ID NO: 143 - protein, 1185 aa, Homo sapiens
MHVHVCVCLCVCIYTSSCVCACVHMCMRDALLAEGRGGGLAAADDFLYLECCKCFSQESQIAMVCQERSQNETYEVKMNNDTEACRATLNLEERRSVAIRSRENVVFVQTDKPTYKPGQKDVNGIAQFFLDTYTFTYPNITLKDPQNNRIFQRQNVTSFRNITQLSFQLISEPMFGDYWIVVKRNSRETVTHQFAVKRYVLPKFEVTVNAPQTVTISDDEFQVDVCAYNFGQPVQGETQIRVCREYFSSSNCEKNENEICEQFIAQVQTNLDIFTLLCSSFLTVMQISEKTSVFITQLLGTVNFENMDTFYRRGISYFGQLKFSDPNNVPMVNKLLQLELNDEFIGNYTTDENGEAQFSIDTSDIFDPEFNLKVRHQRTEECYLPSWLTPQYLDAHFLVSRFYSRTNSFLKIVPEPKQLECNQQKVVTVHYSLNSEAYEDDSNVKFFYLNGNFSFPISISADLAPAAVLFVYTLHPSGEIVADSVRFQVDKCFKHKVNIKFSNEQGLPGSNASLCLQAAPVLFCALRAVDRNVLLLKSEQQLSAESVSSLYNMVPSIEPYGYFYHGLNLDDGKEDPCIPQRDMFYNGLYYTPVSNYGDGDIYNIVRVRSLRILENIIQTVRTNFPETWMWDLVSVSSSGSANLSFLIPDTITQWEASGFCVNGDVGFGISSTTTLEVSQPFFIEIASPFSVVQNEQFDLIVNVFSYRNTCVEVSYIWECLPGKVNITVVAESKQSSACPNEGMEQQKLNWKDTVVQSFLVEFLFLGDILGLALQNLVVLQMPYGSGEQNAALLASDTYVLDYLKSTEQLTEEVQSKAFFLSILGYQRQLSFKNSDGSYSVFWQQSQKGSIWLSALTFKTLERMKKYVFIDENVQKQTLIWLSSQQKTSGCFKNDGQLFNHALRNALFCLEAALDSGVTNGYNHAILAYAFALAGKEKQVESLLQTLDQSAPKLSKRYYWERERKPKTEEFPSFIPWAPSAQTEKSCYVLLAVISRKIPDLTYASKIVQWLAQRMNSHGGFSSNQVINVGLILIAARGEEGLFSKDQNTVTFSSEGSSEIFQVNGHNRLLVQRSEVTQAPGEYTVDVEGHGCTFIQIFRYTGIRNKSSMVVIDVKMLSGFTPTMSSIEEVNNRSLIFQHKDSYIEYKRADSFPFSVEQSNLVFNIQPAPAMVYDYYEKGRQATAMP

SEQ ID NO: 144 - protein, 1432 aa, Homo sapiens
MTGSHLSHDSWQYVLLIPSVLQEGSLDKACAQLFNLTESVVLTVSLNYGEVQTKIFEENVTGENFFKCISFEVPQARSDPLAFITFSAKGATLNLEERRSVAIRSRENVVFVQTDKPTYKPGQKDVNGIAQFFLDTYTFTYPNITLKDPQNNRIFQWQNVTSFRNITQLSFQLISEPMFGDYWIVVKRNSRKTVTHQFAVKRYVLPKFEVTVNAPQTVTISDDEFQVDVCAKYTFGQPVQGKTQIRVCREYFSSSNCEKNDNEICEQFIAQLENGCVSQIVNTKVFQLYRSGLFMTFHVAVIVTEFGTGNYCMQISEKTSVFITQLLGTVNFENMDTFYRRGISYFGTLKFSDPNNVPMVNKLLQLELNDEFIGNYTTDENGEAQFSIDTSDIFDPEFNLKATYVRPKSCYLPSWLTPQYLDAHFLVSRFYSRTNSFLKIVPEPKQLECNHQKVVTVHYSLNSEAYEDDSNVKFFYLVSLSHDSGNFQHRISSSPIYTAWNGNFSFPLSISADLAPAAVLFVYTLHPSGEIVADSVRFQVDKCFKHKVNIKFSNEQGLPGSNASLYLQAAPVLFCALGAVDGNVLLLKSEQQLSAESVYNMVPSIEPYGYFYHGLNLDDGKEDPCIPQRDMFYNGLYYTPVSNYGDGDIYNIVRNMGLKVFTNLHYRKPEVCVMERRLPLPKPLYLETENYGPMHSVPSRIACRGENADYVEQAIIQTVRTNFPETWMWDLVSVDSSGSANLSFLIPDTITQWEASGFCVNGDVGFGISSTTTLEVSQPFFIEIASPFSVVQNEQFDLIVNAFSYLNTCVEISVQVEESQNYEANINTWKINGSEVIQAGGRKTNIWTIIPKKLGKVNITVVAESKQSSACPNEGMEQQKLNWKDTVVKSFLVEPEGIEKERTQSFLICTEGAKASKQGVLDLPNDVVEGSARGFFTVVGDILGLAMQNLVVLQMPYGGGEQNAALLASDTYVLDYLKSTEQLTEEVQSKAFFLLSNGYQRQLSFKNSDGSYSVFWQQNQKGSIWLSALTFKTLERMKKFVFIDENVQKQTLIWLSSQQKTSGCFKNDGQLFNHAWEGGDEEDISLTAYVVGMFFEAGLNSTFPALRNALFCLEAALDSGVTNGYNHAILAYAFALAGKEKQVESLLQTLDQSATKLNNVIYWERERKPKTEEFPSFIPWAPSAQTEKSCYVLLAVISRKIPDLTYASKIVQWLAQQMNSHGGFSSNQVINVGLILIAICGEEGLFSKNQNTVTFSSEGSSEIQFNGHNRLLVQRSEVTQAPGQYTVDVEGRGCTFIQATLKYNVLLPKKASGFSLSLEIVKNYSLTVFDLTVNLKYTGIRNKSSMVVIDVKMLSGFTPTMSSIEELENKGQVMKTEVKNDHVLFYLENVFGRADSFTFSVEQSNLVFNIQPAPGMVYDYYEKDGEAFLLTN

SEQ ID NO: 145 - protein, 7 aa, Homo sapiens
DQDATMS

SEQ ID NO: 146 - protein, 16 aa, Homo sapiens
DTDDLKQLANGVDRYI

SEQ ID NO: 147 - protein, 7 aa, Homo sapiens
DKVSNQT

SEQ ID NO: 148 - protein, 8 aa, Homo sapiens
YAYYNLEE

SEQ ID NO: 149 - protein, 12 aa, Homo sapiens
WRNGKVGLSGMA

SEQ ID NO: 150 - protein, 16 aa, Homo sapiens
LRADLEKLTSLSDRYV

SEQ ID NO: 151 - protein, 7 aa, Homo sapiens
DSVPTSR

SEQ ID NO: 152 - protein, 8 aa, Homo sapiens
YDYYNPER

SEQ ID NO: 153 - protein, 12 aa, Homo sapiens
WRNGKVGLSGMA

SEQ ID NO: 154 - protein, 16 aa, Homo sapiens
LRADLEKLTSLSDRYV

SEQ ID NO: 155 - protein, 7 aa, Homo sapiens
DSVPTSR

SEQ ID NO: 156 - protein, 8 aa, Homo sapiens
YDYYNPER

SEQ ID NO: 157 - protein, 12 aa, Homo sapiens
SREESSSGSSHA

SEQ ID NO: 158 - protein, 16 aa, Homo sapiens
NEEDLKALVEGVDQLF

SEQ ID NO: 159 - protein, 7 aa, Homo sapiens
NSIPSSD

SEQ ID NO: 160 - protein, 8 aa, Homo sapiens
YEYHRPDK

SEQ ID NO: 161 - protein, 12 aa, Homo sapiens
SYTGSRSASNMA

SEQ ID NO: 162 - protein, 14 aa, Homo sapiens
LKPTVKMLERSNHV

SEQ ID NO: 163 - protein, 7 aa, Homo sapiens
DKVSNQT

SEQ ID NO: 164 - protein, 8 aa, Homo sapiens
YDYYETDE

SEQ ID NO: 165 - protein, 12 aa, Homo sapiens
SYTGNRPASNMV

SEQ ID NO: 166 - protein, 14 aa, Homo sapiens
LKPTVKMLERSSSV

SEQ ID NO: 167 - protein, 7 aa, Homo sapiens
EQVTNQT

SEQ ID NO: 168 - protein, 8 aa, Homo sapiens
YDYYETDE

SEQ ID NO: 169 - protein, 11 aa, Homo sapiens
SFSGPGRSGMA

SEQ ID NO: 170 - protein, 13 aa, Homo sapiens
MVPSEAISLSETV

SEQ ID NO: 171 - protein, 7 aa, Homo sapiens
DSVNETQ

SEQ ID NO: 172 - protein, 8 aa, Homo sapiens
VDYYEPRR

SEQ ID NO: 173 - protein, 11 aa, Homo sapiens
RWLHAGSSNMA

SEQ ID NO: 174 - protein, 16 aa, Homo sapiens
DIESLEQLLLDKHMGM

SEQ ID NO: 175 - protein, 7 aa, Homo sapiens
DEIPSRC

SEQ ID NO: 176 - protein, 8 aa, Homo sapiens
YDYYEPAF

SEQ ID NO: 177 - protein, 12 aa, Homo sapiens
SYVGSRSSSNMA

SEQ ID NO: 178 - protein, 14 aa, Homo sapiens
MEGTNQLLLQQPLV

SEQ ID NO: 179 - protein, 7 aa, Homo sapiens
DELIKNT

SEQ ID NO: 180 - protein, 8 aa, Homo sapiens
YDYYLPDE

SEQ ID NO: 181 - protein, 12 aa, Homo sapiens
RYTGIRNKSSMV

SEQ ID NO: 182 - protein, 14 aa, Homo sapiens
TMSSIEEVNNRSLI

SEQ ID NO: 183 - protein, 5 aa, Homo sapiens
EYKRA

SEQ ID NO: 184 - protein, 8 aa, Homo sapiens
YDYYEKGR

SEQ ID NO: 185 - protein, 12 aa, Homo sapiens
KYTGIRNKSSMV

SEQ ID NO: 186 - protein, 14 aa, Homo sapiens
TMSSIEELENKGQV

SEQ ID NO: 187 - protein, 5 aa, Homo sapiens
EYKRA

SEQ ID NO: 188 - protein, 8 aa, Homo sapiens
YDYYEKGR

SEQ ID NO: 189 - protein, 144 aa, Homo sapiens
AKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHP

SEQ ID NO: 190 - protein, 89 aa, Homo sapiens
SVQLTEKRMDKVGKYPKELRKCCEDGMRENPMRFSCQRRTRFISLGEACKKVFLDCCNYITELRRQHARASHLGLARSNLDEDIIAEEN

SEQ ID NO: 191 - protein, 185 aa, Homo sapiens
DMKNTTCQDLQIEVTVKGHVEYTMEANEDYEDYEYDELPAKDDPDAPLQPVTPLQLFEGRRNRRRREAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGA

SEQ ID NO: 192 - protein, 92 aa, Homo sapiens
NVNFQKAINEKLGQYASPTAKRCCQDGVTRLPMMRSCEQRAARVQQPDCREPFLSCCQFAESLRKKSRDKGQAGLQRALEILQEEDLIDEDD

SEQ ID NO: 193 - protein, 185 aa, Homo sapiens; identical to SEQ ID NO: 191

SEQ ID NO: 194 - protein, 92 aa, Homo sapiens; identical to SEQ ID NO: 192

SEQ ID NO: 195 - protein, 143 aa, Homo sapiens
STSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYST

SEQ ID NO: 196 - protein, 85 aa, Homo sapiens
TLQKKIEEIAAKYKHSVVKKCCYDGACVNNDETCEQRAARISLGPRCIKAFTECCVVASQLRANISHKDMQLGRLHMKTLLPVSK

SEQ ID NO: 197 - protein, 140 aa, Homo sapiens; identical to SEQ ID NO: 3

SEQ ID NO: 198 - protein, 39 aa, Homo sapiens; identical to SEQ ID NO: 4

SEQ ID NO: 199 - protein, 142 aa, Homo sapiens
EKEDSPFALKVQTVPQTCDGHKAHTSFQISLTISYTGNRPASNMVIVDVKMVSGFIPLKPTVKMLERSSSVSRTEVSNNHVLIYVEQVTNQTLSFSFMVLQDIPVGDLKPAIVKVYDYYETDESVVAEYIAPCSTDTEHGNV

SEQ ID NO: 200 - protein, 49 aa, Homo sapiens
SVIPSVSAGAVGQGYYGAGLGVVERPYVPQLGTYNVIPLNNEQSSGPVP

SEQ ID NO: 201 - protein, 147 aa, Homo sapiens
SIQNQEAFDLDVAVKENKDDLNHVDLNVCTSFSGPGRSGMALMEVNLLSGFMVPSEAISLSETVKKVEYDHGKLNLYLDSVNETQFCVNIPAVRNFKVSNTQDASVSIVDYYEPRRQAVRSYNSEVKLSSCDLCSDVQGCRPCEDGA

SEQ ID NO: 202 - protein, 31 aa, Homo sapiens
GVYDNAEYAERFMEENEGHIVDIHDFSLGSS

SEQ ID NO: 203 - protein, 171 aa, Homo sapiens
PVAKPAFQLLVSLQEPEAQGRPPPMPASAAEGSRGDWPPADDDDPAADQHHQEYKVMLEVCTRWLHAGSSNMAVLEVPLLSGFRADIESLEQLLLDKHMGMKRYEVAGRRVLFYFDEIPSRCLTCVRFRALRECVVGRTSALPVSVYDYYEPAFEATRFYNVSTHSPLARE

SEQ ID NO: 204 - protein, 32 aa, Homo sapiens
GGLYTDEAVPAFQPHTGSLVAVAPSRHPPRTE

SEQ ID NO: 205 - protein, 134 aa, Homo sapiens
PTNMKTFSLSVEIGKARCEQPTSPRSLTLTIHTSYVGSRSSSNMAIVEVKMLSGFSPMEGTNQLLLQQPLVKKVEFGTDTLNIYLDELIKNTQTYTFTISQSVLVTNLKPATIKVYDYYLPDEQATIQYSDPCE

SEQ ID NO: 206 - protein, 33 aa, Homo sapiens
SHRSPEYSTAMGAGGGHPEAFESSTPLHQAEDS

SEQ ID NO: 207 - protein, 90 aa, Homo sapiens
IFRYTGIRNKSSMVVIDVKMLSGFTPTMSSIEEVNNRSLIFQHKDSYIEYKRADSFPFSVEQSNLVFNIQPAPAMVYDYYEKGRQATAMP

SEQ ID NO: 208 - skipped

SEQ ID NO: 209 - protein, 125 aa, Homo sapiens
PKKASGFSLSLEIVKNYSLTVFDLTVNLKYTGIRNKSSMVVIDVKMLSGFTPTMSSIEELENKGQVMKTEVKNDHVLFYLENVFGRADSFTFSVEQSNLVFNIQPAPGMVYDYYEKDGEAFLLTN

SEQ ID NO: 210 - protein, 44 aa, Homo sapiens
VMERRLPLPKPLYLETENYGPMHSVPSRIACRGENADYVEQAII

SEQ ID NO: 211 - protein, 14 aa, Homo sapiens
YSDASDCHGEDSQA

SEQ ID NO: 212 - protein, 21 aa, Homo sapiens
NYKDRSPCYGYQWVSEEHEEA

SEQ ID NO: 213 - protein, 22 aa, Homo sapiens
NRHNVYINGITYTPVSSTNEKD

SEQ ID NO: 214 - protein, 11 aa, Homo sapiens
LSRVLNCDKQE

SEQ ID NO: 215 - protein, 21 aa, Homo sapiens
FTVHPNLCFHYSWVAEDHQGA

SEQ ID NO: 216 - protein, 20 aa, Homo sapiens
RPFFIHNGAIYVPLSSNEAD

SEQ ID NO: 217 - protein, 13 aa, Homo sapiens
YFSSSNCEKNENE

SEQ ID NO: 218 - protein, 21 aa, Homo sapiens
RHQRTEECYLPSWLTPQYLDA

SEQ ID NO: 219 - protein, 22 aa, Homo sapiens
PQRDMFYNGLYYTPVSNYGDGD

SEQ ID NO: 220 - protein, 13 aa, Homo sapiens
YFSSSNCEKNENE

SEQ ID NO: 221 - protein, 21 aa, Homo sapiens
TYVRPKSCYLPSWLTPQYLDA

SEQ ID NO: 222 - protein, 22 aa, Homo sapiens
PQRDMFYNGLYYTPVSNYGDGD

SEQ ID NO: 223 - protein, 5 aa, synthetic construct
  REGION 1..5: note = sequence can be repeated 1, 2, 3, 4, 5, or 6 times
GGGGS

(19) United States
(12) Patent Application Publication
(10) Pub. No.: US 2024/0374745 A1
(43) Pub. Date: Nov. 14, 2024

(54) BIOPHARMACEUTICAL PRODRUG PLATFORM BASED ON PROTEIN CONFORMATIONAL CHANGE

(71) Applicant: Aarhus Universitet, Aarhus C (DK)

(72) Inventors: Seandean Lykke Harwood, Aarhus C (DK); Jan Johannes Enghild, Ronde (DK)

(21) Appl. No.: 18/790,586

(22) Filed: Jul. 31, 2024

Related U.S. Application Data
(63) Continuation of application No. PCT/EP2023/052280, filed on Jan. 31, 2023.

(30) Foreign Application Priority Data
Jan. 31, 2022 (EP) 22154258.2

(51) Int. Cl. A61K 47/64; A61K 47/65

(52) U.S. Cl. CPC A61K 47/64; A61K 47/65

(57) ABSTRACT

The present invention relates to proteinaceous prodrug constructs, e.g., proteinaceous fusion constructs that comprise a complement 3- and pregnancy zone protein-like, alpha-2-macroglobulin domain-containing (CPAMD) protein (e.g., A2M) and one or more drugs and function as protease-activatable prodrugs.

### CROSS-REFERENCE TO RELATED APPLICATIONS

This application is a Continuation application of International Patent Application No. PCT/EP2023/052280, filed on Jan. 31, 2023, which claims the benefit of, and priority to, European Patent Application Serial No. 22154258.2, filed on Jan. 31, 2022, the contents of each of which is incorporated herein in their entireties for all purposes.

### SEQUENCE LISTING

This application is being filed with an electronically submitted Sequence Listing file in XML format; the file is named SEQUENCE LIST 77582PC01 26 01, created on Jan. 1, 2023, which has a size of 426 kilobytes; the contents of which are incorporated herein by reference in their entirety.

### TECHNICAL FIELD OF THE INVENTION

The present invention relates to proteinaceous prodrug constructs, e.g., proteinaceous fusion constructs that comprise a complement 3- and pregnancy zone protein-like, alpha-2-macroglobulin domain-containing (CPAMD) protein (e.g., A2M) and one or more drugs and function as protease-activatable prodrugs.

### BACKGROUND OF THE INVENTION

When a biopharmaceutical drug is administered to patients in a form that is initially active, it can exert its biological effect both in diseased and healthy tissues. Drug effects in healthy tissue may be detrimental to patient health, quality-of-life, and/or treatment efficacy. One strategy to minimize these side effects is the derivation of drugs into a prodrug form that is initially inactive and first becomes active in the diseased environment.

Proteases are enzymes that catalyze the hydrolysis of peptide bonds in other proteins. There are over 600 known human proteases and the majority are closely regulated under normal circumstances. However, in many diseases specific proteases become dysregulated and have an increased activity relative to healthy conditions. Many protease-activated prodrug technologies for biopharmaceutical drugs have been developed, where most are based on antibodies. Several technologies use masking moieties which block the antibody's antigen-binding region (paratope), preventing it from binding to its cognate epitope on the target antigen and thus rendering it inactive. The masking moiety is attached to the antibody by a linker incorporating a protease-cleavable site. Cleavage of the linker by proteases separates the antibody from the masking moiety, liberating the paratope and restoring the antibody's activity. Masking moieties may be designed to specifically bind to the antibody paratope (e.g., using phage display-derived peptides as in CytomX's Probody technology) or may sterically encompass the paratope sufficiently to sequester it without any specific interactions (e.g. using long, bulky peptides as in Amunix's XPAT technology). Another approach prevents functional VH/VL domain pairing in the prodrug by incorporating inactive VH and VL domains, attached to the antibody by a protease-sensitive linker (e.g. Maverick's COBRA technology). The linker's cleavage removes the inactive domains and enables the correct VH/VL pairing in the activated prodrug.

These diverse prodrug technologies all have their advantages and disadvantages. For example, CytomX's Probody minimizes the use of non-human and potentially immunogenic sequences, but is not modular and requires the identification of affine masking moieties for every antibody incorporated into their platform. Amunix's XPAT platform does not require specific mask/antibody interactions and furthermore has a difference in circulatory half-life before and after the prodrug's activation, but this is accomplished by using long non-human peptides. Hence, new prodrug technologies combining the key advantages of multiple technologies are needed and would constitute meaningful advancement of the field.

### SUMMARY OF THE INVENTION

The present invention relates to provision of proteinaceous prodrug constructs (e.g., proteinaceous fusion constructs) that address one or more of the above-mentioned problems associated with existing proteinaceous prodrug platforms.

In particular, the proteinaceous prodrug constructs (e.g., the proteinaceous fusion constructs) described herein enable specific drug delivery and controllable activity. This is achieved by the proteinaceous prodrug construct undergoing a conformational change through which it is able to control the activity of one or more drugs comprised in it. In the native (uncleaved) state of the proteinaceous prodrug construct, the one or more drugs are not exposed and thus, inactive. In the active (cleaved) state of the proteinaceous prodrug construct, the drug is exposed and able to interact with its target.

The conformational change is triggered by the cleavage of a protease cleavage site comprised in the proteinaceous prodrug constructs of the invention. A cleavage site comprised within a proteinaceous prodrug construct can modified to control where the drug becomes exposed. Depending on the cleavage site, the drug can be exposed only at the location where proteases recognizing that cleavage site are present. When a specific protease is present and cleaves the cleavage site, the conformation of the proteinaceous prodrug construct is changed from “native” to “active”. Accordingly, the present invention provides proteinaceous prodrug constructs (e.g., proteinaceous fusion constructs), where the activity and specificity can be controlled and directed towards a specific area (e.g., a particular tissue) of a subject in need of treatment with such constructs.

Specifically, the present invention relates to a proteinaceous prodrug construct comprising (a) a complement 3- and pregnancy zone protein-like, alpha-2-macroglobulin domain-containing (CPAMD) protein or a fragment thereof, and (b) one or more drugs, wherein (i) the CPAMD protein or fragment thereof comprises (1) a bait region with at least one protease cleavage site, and (2) a Receptor Binding Domain (RBD), (ii) the one or more drugs are positioned inside or in the vicinity of the RBD, and (iii) the CPAMD protein or fragment thereof shields the one or more drugs and is capable of altering conformation upon proteolytic cleavage of the at least one protease cleavage site, making the one or more drugs accessible.

In some embodiments, the one or more drugs is positioned inside or in the vicinity of any one of loops 1-4 of the RBD. In some embodiments, the proteinaceous prodrug construct is a fusion protein. In some embodiments, the one or more drugs are positioned inside any one of loops 1-4 of the RBD. In some embodiments, the loop is loop 1. In some embodiments, the loop is loop 2. In some embodiments, the loop is loop 3. In some embodiments, the loop is loop 4. In some embodiments, the loop is modified, in relation to a wildtype loop sequence, by addition, substitution or deletion of one or more amino acids to accommodate the one or more drugs. In some embodiments, the one or more drugs replace one or more amino acids of the loop.

In some embodiments, the one or more drugs is positioned in the vicinity of any one of loops 1-4 of the RBD. In some embodiments, the loop is loop 1. In some embodiments, the loop is loop 2. In some embodiments, the loop is loop 3. In some embodiments, the loop is loop 4. In some embodiments, the proteinaceous prodrug construct comprises a first interaction domain and the one or more drugs comprise a second interaction domain, wherein the first and second interaction domains form a complex positioning the one or more drugs in the vicinity of the loop. In some embodiments, the first interaction domain and the second interaction domain form a coiled coil structure. In some embodiments, the first interaction domain is a tag or epitope sequence within the loop and the second interaction domain is a functional fragment of a receptor or antibody that is capable of binding specifically to the tag or epitope sequence.

In some embodiments, the proteinaceous prodrug construct is capable of forming a multimer (e.g., a dimer or tetramer). In some embodiments, multimer formation occurs via a LNK region of the CPAMD protein. In some embodiments, the multimer is a tetramer formed by two disulfide-bridged dimers.

In some embodiments, the CPAMD protein is human CPAMD protein, or a functional variant, fragment, or homolog thereof, e.g., a mammalian CPAMD protein. In some embodiments, the CPAMD protein, or a functional variant, fragment, or homolog thereof, has an amino acid sequence that is at least about 70%, at least about 80%, or at least about 90% identical to any one of the full-length CPAMD protein sequences set forth in Table 1.

In some embodiments, the CPAMD protein, or a functional variant, fragment, or homolog thereof, has an amino acid sequence that is at least about 91%, at least about 92%, at least about 93%, at least about 94%, at least about 95%, at least about 96%, at least about 97%, at least about 98%, at least about 99% identical to, or identical to, any one of the full-length CPAMD protein sequences set forth in Table 1.

In some embodiments, the CPAMD protein is selected from A2M, PZP, Ovostatin 1, Ovostatin 2, CPAMD1, CPAMD2, CPAMD3, CPAMD4, CPAMD7, CPAMD8, CPAMD9, and functional homolog thereof. In particular embodiments, the CPAMD protein is selected from A2M, PZP, Ovostatin 1, and Ovostatin 2, and functional homolog thereof.

In some embodiments, the CPAMD protein is human A2M, or a functional homolog thereof, e.g., a mammalian A2M. In some embodiments, the functional homolog of human A2M has an amino acid sequence that is at least about 70%, at least about 80%, or at least about 90% identical to the amino acid sequence set forth in SEQ ID NO: 1.

In some embodiments, the human A2M has an amino acid sequence that is at least about 91%, at least about 92%, at least about 93%, at least about 94%, at least about 95%, at least about 96%, at least about 97%, at least about 98%, at least about 99% identical to, or identical to, the amino acid sequence set forth in SEQ ID NO: 1.

In some embodiments, the one or more drugs are positioned within a region comprising amino acid 1368-1379, 1392-1404, 1420-1426 or 1450-1457 of human A2M. In some embodiments, the one or more drugs are positioned between amino acids 1402 and 1403 of human A2M. In some embodiments, the one or more drugs replace amino acids 1392-1403, 1393-1395, or 1393-1402 of human A2M.

In some embodiments, the one or more drugs are selected from the group consisting of: an antigen-targeting moiety (e.g., a single-chain or domain antibody), a receptor ligand (e.g., a cytokine), the extracellular region of a cell surface receptor, the extracellular region of a cell surface ligand, and a receptor agonist.

In some embodiments, the bait region is modified to be selectively cleaved by one or more proteases. In some embodiments, the one or more proteases are selected from one or more serine-, cysteine-, aspartic- and/or metalloproteinases. In some embodiments, the bait region has been modified to be free from protease cleavage sites recognized by human proteases except for a single cleavage site.

In some embodiments, the modified bait region comprises an engineered amino acid sequence that is flexible and/or hydrophilic. In some embodiments, the engineered amino acid sequence comprises a sequence of glycine, serine, alanine, threonine, and/or proline residues. In some embodiments, the engineered amino acid sequence comprises a combination of glycine, serine, and/or alanine residues. In some embodiments, the engineered amino acid sequence replaces all or a portion of a wildtype bait region. In some embodiments, the engineered amino acid sequence replaces all of the wildtype bait region and has a length equivalent to the wildtype bait region.

In some embodiments, the one or more drugs is an antibody, or antigen-binding fragment thereof, that specifically binds to an antigen as an antagonist. In some embodiments, the one or more drugs is an antibody, or antigen-binding fragment thereof, that specifically binds to an antigen as an agonist.

In some embodiments, the one or more drugs is an antibody, or antigen-binding fragment thereof, that specifically binds to antigen selected from the group consisting of IL-2, EGFR, PDL-1, PD-1, CTLA-4, CD3γε, 4-1BB, IL-2Rα, and TNFα.

In some embodiments, the one or more drugs is an antibody, or antigen-binding fragment thereof, that specifically binds to an antigen selected from the group consisting of BTLA, OX40, LAG3, NRP1, VEGF, HER2, CEA, CD19, CD20, Amyloid beta, HER3, IGF-1R, MUC1, EpCAM, CD22, VEGFR-2, PSMA, GM-CSF, CXCR4, CD30, CD70, FGFR2, BCMA, CD44, ICAM-1, Notch1, MHC, CD28, IL-1R1, TCR, Notch3, FGFR3, TGF-β, TGFBR1, TGFBR2, CD109, GITR, CD47, Alpha-synuclein, CD26, LRP1, CD52, IL-4Rα, VAP-1, EPO Receptor, Integrin av, TIM-3, Grp78, LIGHT, TLR2, TLR3, PAR-2, NRP2, GLP-1 receptor, Hedgehog, and Syndecan 1.

In some embodiments, the one or more drugs are selected from the group consisting of Atezolizumab, EgA1, Ipilimumab, Nivolumab, KN035, Urelumab, Foralumab, Muromonab, Adalimumab, and therapeutically active antigen-binding fragments or variants of each. In some embodiments, the one or more drugs are selected from the group consisting of ANB032, rosnilimab, LY3361237, Encelimab, Cobolimab, Imsidolimab, Dostarlimab, and therapeutically active antigen-binding fragments or variants of each.

In some embodiments, the one or more drugs is a cytokine, or a therapeutically active fragment or variant thereof, selected from the group consisting of IL1, IL1alpha, IL1beta, IL2, IL3, IL4, IL6, IL7, IL8, IL9, IL10, IL11, IL12, IL13, IL14, IL15, IL16, IL17, 118, IL19, IL20, IL21, IL22, IL23, IL24, IL25, IL26, IL27, IL28, IL29, IL30, IL31, IL32, IL33, IL34, IL35, IL36, GM-CSF, TGF-β, CSF-1, insulin, GLP-1, HGH, VEGF, PDGF, BMP, EPO, G-CSF, IL-11, IFN-α, IFN-β and IFN-γ.

In some embodiments, the proteinaceous prodrug construct is encoded by an amino acid sequence selected from the group consisting of: SEQ ID NO: 5, SEQ ID NO:7, SEQ ID NO: 9, SEQ ID NO:11, SEQ ID NO:13, SEQ ID NO: 15, and SEQ ID NO:17, SEQ ID NO: 19, SEQ ID NO:21, SEQ ID NO:23, or SEQ ID NO:25.

The invention also provides nucleic acids encoding a proteinaceous prodrug construct according to the invention. Vectors comprising these nucleic acids are also provided. In such vectors, the nucleic acid encoding the proteinaceous prodrug construct may be operatively linked to a promotor, and optionally, additional regulatory sequences that regulate expression of the nucleic acid. Host cell comprising such vectors are also provided. In some embodiments, the host cell is a bacteria or eukaryote, e.g., a mammalian cell.

The invention also relates to the therapeutic use of the proteinaceous prodrug constructs of the invention as well as their use in the manufacture of a medicament for treating a disease or disorder in a subject in need of such treatments.

In some embodiments, the proteinaceous prodrug constructs of the invention find use in methods of treating or preventing a disease or disorder in a subject in need of such treatment, wherein the method comprises administering a therapeutically effective amount of a proteinaceous prodrug construct, a nucleic acid, a vector, or a host cell of the invention to the subject.

In some embodiments, the disease or disorder is a disease or disorder of the nervous system, the eye, the circulatory system, the respiratory system, the digestive system, or the skin. In some embodiments, the disease or disorder is a neoplasm, a blood disorder, a metabolic disorder, an autoimmune disease, an immunodeficiency, or an infectious disease. In some embodiments, the neoplasm is a cancer selected from brain cancer, glioblastoma, lung cancer, colorectal cancer, skin cancer, malignant melanoma, pancreas cancer, bladder cancer, liver cancer, breast cancer, eye cancer, and prostate cancer, the cancer is a haematological cancer, such as selected from the group consisting of multiple myeloma, acute myeloblastic leukemia, chronic myelogenic leukemia, acute lymphoblastic leukemia, and chronic lymphocytic leukemia, or the cancer is malignant melanoma, breast cancer, non-small cell lung cancer, pancreatic cancer, head & neck cancer, liver cancer, sarcoma, and B cell lymphoma. In some embodiments, the autoimmune disease is selected from arthritis (e.g., rheumatoid arthritis or psoriatic arthritis), multiple sclerosis, systemic lupus erythematosus, and inflammatory bowel disease.

The invention also provides methods for producing a proteinaceous prodrug construct of the invention. Such methods comprise (i) introducing into a host cell an expression vector comprising a nucleic acid encoding the proteinaceous prodrug construct, (ii) growing the host cell under conditions that allow for expression of the proteinaceous prodrug construct from the vector, and (iii) purifying the proteinaceous prodrug construct. The nucleic acid is typically operatively linked to a promotor, and optionally, one or more additional regulatory sequences that regulate expression of the nucleic acid.

### BRIEF DESCRIPTION OF THE FIGURES

The following figures illustrate the invention with proteinaceous prodrug constructs that comprise alpha-2-macroglobulin (A2M) as a CPAMD protein. A person of skill in the art of proteinaceous prodrug design will appreciate that other CPAMD proteins can take the place of A2M.

FIG. 1A shows a schematic overview of a proteinaceous prodrug construct (1), e.g., a fusion protein, comprising a CPAMD protein (2), e.g., A2M, fused to one or more drugs (e.g., one or more nanobodies) (3) positioned inside or in the vicinity of the RBD domain of the CPAMD protein. The one or more drugs (3) are inaccessible when the bait region of the CPAMD protein has not been proteolytically cleaved (inactive or “native” conformation I). The one or more drugs (3) are accessible when the bait region is cleaved by a protease (4) (active conformation II). When the protease (4) cleaves the “bait region”, the protease (4) is trapped inside the proteinaceous prodrug construct (1). FIG. 1B shows a schematic overview of the different fusion strategies of the CPAMD protein (e.g., A2M) and the drug.

FIG. 2 shows native PAGE (A) and SDS-PAGE (B) analysis of wildtype A2M and fusions constructs with A2M and antibody scFvs from Atezolizumab, Ipilimumab, and Nivolumab, as noted. Before analysis, samples were treated with methylamine (MA) or thermolysin, as indicated. (C) A schematic of the domain organization of A2M-antibody constructs, showing the size of products generated by thiol ester autolysis and bait region cleavage.

FIG. 3 shows conformational dependence of antigen binding by A2M-antibodies, measured by biolayer interferometry. (A) The interaction between A2M-Atezolizumab (purified by one round of depletion using a PD-L1 resin, see Example 4) and immobilized PD-L1-hFc. The control and methylamine-treated A2M-Atezolizumab show a ≈149-fold difference in their effective concentration, calculated from the fitted k_{obs} values for their association. (B) The interaction between A2M-EgA1 (purified by two rounds of depletion using an LRP1 resin, see Example 4) and immobilized EGFR-hFc. The control and methylamine-treated or thermolysin-treated samples show a ≈63-fold difference in their effective concentration. (C) The interaction between A2M-Ipilimumab (purified by three rounds of depletion using an LRP1 resin) and immobilized CTLA-4-hFc. (D) The interaction between A2M-Nivolumab (purified by three rounds of depletion using an LRP1 resin) and immobilized PD-1-hFc. (E) The interaction between A2M-KN035 (not enriched for native A2M) and immobilized PD-L1-hFc. (F) The interaction between A2M-Urelumab (purified by three rounds of depletion using an LRP1 resin) and immobilized 4-1BB-hFc. (G) The interaction between A2M-Foralumab (not enriched for native A2M) and immobilized CD3γε-hFc. (H) The interaction between A2M-Muromonab (not enriched) and immobilized CD3γε-hFc. In panels G and H, the +thermolysin sensorgram has the signal from a biosensor associating with thermolysin only subtracted, due to the low-intensity responses. (I) The interaction between A2M-Adalimumab (not enriched) and immobilized TNFα.

FIG. 4 shows enrichment of native A2M-antibodies using affinity depletion. (A) A2M-Atezolizumab was depleted using a resin coated with its cognate antigen, PD-L1. One round of depletion was performed. Biolayer interferometry was then used to compare antigen binding of the untreated sample before and after depletion. (B-D) A2M-Nivolumab, A2M-Ipilimumab, and A2M-Urelumab were depleted using LRP1-coated resin. Three rounds of depletion were performed for each A2M-antibody, after which biolayer interferometry was used to compare their antigen binding before and after depletion. (E) A2M-Ipilimumab was depleted by three rounds with Protein L resin and biolayer interferometry was used to compare its antigen binding before and after.

FIG. 5 shows immune checkpoint blockade by A2M-Atezolizumab in a cell bioassay of PD-1/PD-L1 blockade. PD-1⁺ Jurkat T cells with a NFAT-driven luciferase gene to report NFκB signaling were co-cultured with PD-L1⁺ CHO-K1 cells expressing a TCR agonist, in the presence of a dilution series of A2M-Atezolizumab in its native and methylamine-treated conformations, or Atezolizumab scFv fused to a human Fc region. The luminescence response, with the background from control cells subtracted and the subsequent response normalized to the maximum response, is shown. EC₅₀ curves were fitted using linear regression and the maximum response and EC₅₀ values from fitting are shown for each antibody.

FIG. 6 shows the conformation and functionality of tabula rasa A2M which comprises a bait region that cannot be cleaved by proteases. (A) Sequences of the wildtype, tabula rasa (TR) and TR K704 bait regions. Basic residues (i.e. cleavage sites for trypsin or LysC) are highlighted. (B) Pore-limited native PAGE of A2M incorporating the three given bait region sequences. All constructs originally demonstrated the slow electrophoretic mobility that is characteristic of A2M's native conformation; upon methylamine aminolysis or bait region cleavage, A2M collapses and demonstrates a faster electrophoretic mobility. Wildtype A2M and A2M TR K704 were both collapsed by trypsin, while only A2M TR K704 was collapsed by LysC; A2M TR was not collapsed by either protease. (C) Reducing SDS-PAGE of the same A2M samples as in panel B. The thiol ester-dependent heat-fragmentation bands (TE 120 and TE 60) disappeared upon methylamine treatment. Bait region cleavage of A2M gives its ˜85 and ˜95 N- and C-terminal fragment bands; the C-terminal fragment additionally forms high-MW multimer products through thiol ester-mediated conjugation. When the bait region is not cleavable by trypsin or LysC, A2M can be cleaved outside of the bait region without any activation of its thiol ester. A2M TR K704 forms an intense ˜250 kDa band upon proteolytic activation due to thiol ester-mediated conjugation of the bait region lysine residue.

FIG. 7 shows incorporation of MMP2 substrate sites into tabula rasa A2M. (A) Bait region sequences for wildtype A2M, TR A2M, and four TR bait regions each incorporating a different MMP2 substrate sequence (A21A, B74, C9, and S1). The MMP2 recognition sequence is highlighted in each sequence; cleavage occurs at the N-terminus of the bolded hydrophobic residue. (B) A2Ms with these 6 bait regions were digested by MMP2 and nine other human proteases and cleavage was assessed by SDS-PAGE. Proteases that cleave a bait region are indicated with a + in the case of full cleavage and (+) in the case of partial cleavage (relative to wildtype A2M). The TR bait region was not cleaved by any tested protease, whereas each MMP2 substrate was cleaved by every tested MMP. The TR S1 bait region was not cleaved by proteases other than MMPs, indicating an increased selectivity of inhibition relative to the wildtype bait region. (C-D) Pore-limited native PAGE and reducing SDS-PAGE, respectively, of the six A2Ms with and without MMP2 cleavage. All constructs are similarly bait region-cleaved by MMP2, resulting in a conformational collapse and the appearance of high-MW multimer products in SDS-PAGE, with the exception of A2M TR.

FIG. 8 shows optimization of the production and inhibitory capacity of A2M TR S1. (A) Several modifications of the MMP2 substrate bait region, tabula rasa S1, were tested for their ability to improve the formation of native A2M and its inhibitory capacity towards MMP2. TR S1 QRT4 re-introduces the fourth quarter of the wildtype bait region. Two different S1 positions (with cleavage at position 710 or 703) were tested in TRΔ7, which shortens the TR bait region by seven residues. (B) Pore-limited native PAGE of A2Ms with the indicated bait regions. A2M TR S1 is expressed with a substantial amount of non-native A2M. This non-native A2M could be removed by depletion using LRP1-conjugated resin. Alternatively, the native content was improved in TRΔ7 and TR QRT4. (C) The ability of the indicated A2Ms to inhibit MMP2's digestion of DQ-gelatin was determined. Fitted curves calculated from the experimental data points by linear regression are shown as dotted lines. Error bars show the standard; n=3.

FIG. 9 shows A2M-antibodies incorporating engineered bait regions. (A) Bait region sequences for the wildtype A2M bait region, the shortened MMP2 substrate bait region “TRΔ7 S1 I703” that was described in Example 6, and an additional engineered bait region “TRΔ7 S1 I703 P704.” (B) Pore limited native PAGE and (C) reducing SDS-PAGE of wildtype A2M, A2M-Atezolizumab with a wildtype bait region, and A2M-Atezolizumab with the TRΔ7 S1 I703 bait region. The A2Ms were analyzed untreated, methylamine-treated, or treated by a 0.5:1 or 4:1 molar ratio of MMP2:A2M, as indicated. (D) Biolayer interferometry was used to assess PD-L1 binding by A2M-Atezolizumab with the 3 bait regions shown in panel A, before and after MMP2 cleavage. A biosensor associating with MMP2 only, without A2M-Atezolizumab, is included to account for this background binding. A2M-Atezolizumab with wildtype bait region was additionally cleaved with thermolysin for comparison.

FIG. 10 shows: (A) Reducing SDS-PAGE analysis of purified A2M-PD1. A2M-PD1 is expressed and purified by the same protocol as wildtype A2M or A2M-antibodies, to a high purity. The formation of an internal thiol ester in A2M-PD1 causes heat-induced fragmentation at the thiol ester site under denaturing conditions, generating an N-terminal and C-terminal product band. (B) A2M-PD1 binding to immobilized PD-L1, as assessed by biolayer interferometry. PD-L1 binding by A2M-PD1 without any treatment to change its conformation or after methylamine- or thermolysin-treatment to collapse its conformation is shown. A reference biosensor where thermolysin was added without any A2M-PD1 was included to account for non-specific binding of thermolysin to the biosensor surface, and has been subtracted from the A2M-PD1+thermolysin sensorgram. A2M-PD1 after LRP1 depletion was also included, without treatment and after methylamine treatment.

FIG. 11 shows: (A) Reducing SDS-PAGE analysis of purified A2M-IL2. A2M-IL2 is expressed and purified by the same protocol as wildtype A2M or A2M-antibodies, to a high purity. The formation of an internal thiol ester in A2M-IL2 causes heat-induced fragmentation at the thiol ester site under denaturing conditions, generating an N-terminal and C-terminal product band. (B) A2M-IL2 binding to immobilized IL-2Rα, as assessed by biolayer interferometry. IL-2Rα binding by A2M-IL2 without any treatment to change its conformation or after methylamine- or thermolysin-treatment to collapse its conformation is shown. A2M-IL2 after three rounds of LRP1 depletion was assessed in the same manner.

FIG. 12 shows: (A) The interaction between 5 nM A2M-fusion-EgA1, before and after methylamine treatment, with immobilized human EGFR, measured during one hour association and one hour dissociation using biolayer interferometry. (B) The interaction between 5 nM A2M-iRBD-EgA1, before and after methylamine or thermolysin treatment, with immobilized human EGFR, measured during one hour association and one hour dissociation using biolayer interferometry. (C-E) The interactions between 5 nM of A2M-miRBD-EgA1, A2M-miRBD-KN035, and A2M-miRBD-Atezolizumab, before and after methylamine treatment, with immobilized EGFR or PD-L1, measured during one hour association and one hour dissociation (or two hours of association and 10 minutes dissociation, in the case of A2M-miRBD-Atezolizumab) using biolayer interferometry. (F) The interaction between 10 nM of A2M-tRBD-EgA1, before and after methylamine treatment, with immobilized EGFR, measured during one hour association and dissociation.

FIG. 13 shows: The RBD domain (residues 1335-1474 of SEQ ID NO: 1) of A2M, with emphasis on four proposed sites that can be used for the insertion of drugs to achieve conformation-dependent binding. These sites are residues 1392-1404 or 1391-1405 (loop 2), as demonstrated by the ciRBD, iRBD, miRBD, and tRBD fusion approaches, as well as residues 1368-1379 (loop 1), 1420-1426 (loop 3), and 1450-1457 (loop 4), all of which are flexible linkers between beta strands that are spatially close to 1392-1404 and facing the same direction on the RBD domain. In contrast, residue 1468 defines the position of the inserted drug in the A2M-fusion-EgA1 construct, where conformation-dependent binding was not obtained, indicating that this opposite side of the RBD domain is unsuited to achieve conformation-dependent binding. The RBD domain structure (from PDB accession code 7VON) is represented as a cartoon, while the Ca atoms of the indicated residues are shown as spheres. The RBD domain is shown from two different angles, as indicated.

The present invention will now be described in more detail in the following.

### General

In order for the present invention to be more readily understood, certain terms are first defined below. Additional definitions for the following terms and other terms are set forth throughout the specification.

As used in this specification and the appended claims, the singular forms “a,” “an” and “the” include plural referents unless the context clearly dictates otherwise. For example, “a ribonucleotide” is understood to represent one or more ribonucleotides. As such, the terms “a” (or “an”), “one or more”, and “at least one” can be used interchangeably herein.

Unless specifically stated or obvious from context, as used herein, the term “or” is understood to be inclusive and covers both “or” and “and”. Furthermore, “and/or” where used herein is to be taken as specific disclosure of each of the two specified features or components with or without the other. Thus, the term “and/or” as used in a phrase such as “A and/or B” herein is intended to include “A and B”, “A or B”, “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this specification and embodiments, the words “have” and “comprise”, or variations such as “has”, “having”, “comprises”, or “comprising” will be understood to imply the inclusion of a stated element, feature, or integer, or group of elements, features, or integers, but not the exclusion of any other elements, features, or integers or group of elements, features, or integers. It is further understood that wherever embodiments are described herein with the language “comprising” or “having” of grammatical equivalents thereof, otherwise analogous embodiments described in terms of “consisting of” and/or “consisting essentially of” are also provided.

As used herein, the term “about” refers to an interval of accuracy that a person skilled in the art will understand to still ensure the technical effect of the feature in question. The term indicates a deviation from the indicated numerical value of ±10%. In some embodiments, the deviation is ±5% of the indicated numerical value. In certain embodiments, the deviation is ±1% of the indicated numerical value.

The terms “variant” and “homolog” are used interchangeable to refer to proteins in which at least one function of the reference protein is preserved (e.g., to undergo a conformational change upon cleavage by a protease). In some embodiments, a variant or homolog is at least 70%, at least 80%, at least 85%, at least 90%, at least 91%, at least 92%, at least 93%, at least 94%, at least 95%, at least 96%, at least 97%, at least 98%, or 99% identical to a wildtype version of the reference protein (e.g., a CPAMD protein such as A2M, e.g., human A2M comprising or consisting of the amino acid sequence set forth in SEQ ID NO: 1).

As used herein, the term “fragment” refers to a protein that is truncated (e.g., N-terminally and/or C-terminally) by one or more amino acids or comprises one or more deletions of amino acids while preserving at least one function of the reference protein (e.g., to specifically bind an antigen or receptor, for instance in case of an antibody or cytokine, or to undergo a conformational change upon cleavage by a protease, for instance in case of a CPAMD protein such as A2M).

As used herein, the terms “therapeutic” and “therapeutically active” refer to any pharmaceutical, drug or composition that can be used to treat or prevent a disease, illness, condition or disorder or bodily function.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

### Definitions

Prior to discussing the present invention in further detail, the following terms and conventions will first be defined:

### Alpha-2-Macroglobulin (A2M)

The term “A2M” is to be understood as the human protein A2M (NCBI #9606, Uniprot P01023), or variants or fragments thereof, that comprise (1) a bait region with at least one protease cleavage site, and (2) a Receptor Binding Domain (RBD), and are capable of altering conformation upon proteolytic cleavage of the at least one protease cleavage site. A2M is also known as C3 and PZP-like alpha-2-macroglobulin domain-containing protein 5 (CPAMD5). The amino acid sequence of human A2M is given in SEQ ID NO: 1, with the naturally occurring polymorphisms I1000V and N639D. Unless indicated otherwise, residue numbers that are provided herein to identify specific amino acids or regions of A2M refer to the residues as set forth in SEQ ID NO:1. It will be apparent to the skilled person that the numbering may differ in A2M variants that comprise one or more of the modifications described herein.

### Antigen-Targeting Moiety

The term “antigen-targeting moiety” of the invention includes single chain variable fragment, monoclonal, recombinant, chimeric, humanized, fully human, single-chain, single-domain and/or bispecific antibodies including antibody fragments. Examples of such fragments include Fab F(ab′), F(ab)′, Fv, and sFv fragments. The antibodies may be generated by enzymatic cleavage of full-length antibodies or by recombinant DNA techniques, such as expression of recombinant plasmids containing nucleic acid sequences encoding antibody variable regions.

A “Single-chain Fv”, “sFv” or “scFv” antibody comprises a VH domain and a VL domain in a single polypeptide chain. The VH and VL are typically linked by a peptide linker. Any suitable linker may be used. In some embodiments, the linker is a (GGGGS)n (SEQ ID NO: 223) or a (GGS)n. In some embodiments, n=1, 2, 3, 4, 5, or 6.

The term “single-domain antibody” refers to an antigen-targeting moiety in which one variable domain of an antibody specifically binds to an antigen without the presence of another variable domain. Single domain antibodies include nanobodies.

An antigen is a molecule or a portion of a molecule capable of being bound by an antibody, which is additionally capable of inducing an animal to produce antibody capable of binding to an epitope of that antigen. An antigen can have one or more epitopes. The specific reaction referred to above is meant to indicate that the antigen will react, in a highly selective manner, with its corresponding antibody and not with the multitude of other antibodies, which can be evoked by other antigens.

Monoclonal antibodies (mAbs) contain a substantially homogeneous population of antibodies specific to antigens, which population contains substantially similar epitope binding sites. Such antibodies may be of any immunoglobulin class including IgG, IgM, IgE, IgA, IgD and any subclass thereof. A hybridoma producing a monoclonal antibody of the present invention may be cultivated in vitro, in situ, or in vivo. Production of high titers in vivo or in situ is a preferred method of production.

Chimeric antibodies are molecules in which different portions are derived from different animal species, such as those having a variable region derived from a murine monoclonal antibody and a human immunoglobulin constant region.

The term “chimeric antibody”, as used herein, includes monovalent, divalent or polyvalent immunoglobulins. A monovalent chimeric antibody is a dimer (HL) formed by a chimeric H chain associated through disulfide bridges with a chimeric L chain. A divalent chimeric antibody is tetramer (H2L2) formed by two HL dimers associated through at least one disulfide bridge. A polyvalent chimeric antibody can also be produced, for example, by employing a CH region that aggregates (e.g., from an IgM H chain, or [micro] chain).

Murine and chimeric antibodies, fragments and regions of the present invention may comprise individual heavy (H) and/or light (L) immunoglobulin chains.

Selective binding agents, such as antibodies, fragments, or derivatives, having chimeric H chains and L chains of the same or different variable region binding specificity, can also be prepared by the appropriate association of the individual polypeptide chains.

In some embodiments, the term “antibody” as used herein refers to a single-chain or single-domain antibody.

### CPAMD

The term “CPAMD” is to be understood as the C3 and PZP-like alpha-2-macroglobulin domain-containing protein (CPAMD) family, to which A2M belongs, or a member of such family. An illustrative list of CPAMD proteins is provided in Table 1. In some embodiments, a proteinaceous prodrug construct of the invention may comprise a variant or fragment of a naturally occurring CPAMD protein. Such variants or fragments retain the capability of shielding the one or more drugs and altering their conformation upon proteolytic cleavage of the at least one protease cleavage site comprised in them to make the one or more drugs comprised in the proteinaceous prodrug construct accessible.

### RBD Domain

The terms “RBD” or “RBD domain” is to be understood as the receptor-binding domain of a CPAMD protein (e.g., A2M). In the native human A2M protein, the RBD is located at its C-terminus and spans amino acids 1335-1474 of A2M. Y1452 and Y1453 are involved in the formation of thiol ester groups. The thiol ester groups stabilize the molecule in its “native” conformation.

The amino acid sequence of the RBD domain of native human A2M is given in SEQ ID NO: 3. The RBD domain is also known as the macroglobulin 8 (MG8) domain. In the proteinaceous prodrug constructs described herein, one or more drugs (e.g., a therapeutic peptide, polypeptide or protein) is positioned inside or in the vicinity of the RBD such that the CPAMD protein (e.g., A2M) remains capable of altering conformation upon proteolytic cleavage of at least one protease cleavage site comprised in the bait region of the CPAMD protein.

### Inaccessible

The term “inaccessible” is to be understood as the drug of the proteinaceous prodrug construct possessing a decreased ability to interact with its binding partner when the construct is in a “closed” conformation (not proteolytically cleaved). Thus, the drug is “inaccessible” to its binding partner (e.g., in the case the drug is an antibody such as scFv or nanobody).

Thus, the term “inaccessible” may also be understood as the drug being “inactive”, in an “inactivated state”, or “shielded”.

Thus, in an embodiment,
a. the one or more drugs is inaccessible when the bait region in the CPAMD protein (e.g., A2M) has not been proteolytically cleaved; and
b. the one or more drugs is accessible when the bait region in the CPAMD protein (e.g., A2M) has been proteolytically cleaved.

### Bait Region

The term “bait region” is to be understood as the region of a CPAMD protein (e.g., A2M) that comprises at least one protease cleavage site. In the native human A2M protein, the bait region spans amino acids 690-728 of A2M. The sequence of the bait region of native human A2M is given in SEQ ID NO: 4. The bait region of native human A2M is preferentially cleaved by most proteases, and bait region cleavage triggers A2M's conformational change. The bait region sequence may be modified in order to change the selection of proteases that are able to cleave the bait region and trigger conformational change of the CPAMD protein.

### Biopharmaceutical Moiety

The term “Biopharmaceutical moiety” is to be understood as a protein or fragment of a protein (e.g., a peptide or polypeptide) with therapeutic properties that can be incorporated into a proteinaceous prodrug construct with a CPAMD protein (e.g., A2M) in order to produce a proteolytically activatable prodrug. The term is used interchangeably herein with the term “drug”. Examples of biopharmaceutical moieties include antibody fragments such as single-domain antibodies (e.g. nanobodies) or single-chain variable fragments (scFvs), cytokines, or fragments of cell surface receptors or ligands. Example sequences are given for the EGFR-binding nanobody EgA1 (SEQ ID NO: 27), the scFv from PDL1-binding Atezolizumab (SEQ ID NO: 28), the PDL1-binding nanobody KN035 (SEQ ID NO: 29), the scFv from PD1-binding Nivolumab (SEQ ID NO: 30), the scFv from CTLA-4-binding Ipilimumab (SEQ ID NO: 31), the scFv from CD3-binding Foralumab (SEQ ID NO: 32), the scFv from CD3-binding Muromonab (SEQ ID NO: 33), the scFv from 4-1BB-binding Urelumab (SEQ ID NO: 34), the scFv from TNFα-binding Nivolumab (SEQ ID NO: 35), the IL2 cytokine (SEQ ID NO: 36), or the extracellular region of the PD1 receptor (SEQ ID NO: 39).

Terms such as “biopharmaceutical moiety”, “drug”, “therapeutic peptide”, “therapeutic polypeptide” or “therapeutic protein”, “active agent” are used herein to refer to proteinaceous compounds that can be used to treat or prevent a disease, illness, condition, or disorder of bodily function.

ciRBD

The term “ciRBD” is to be understood as proteinaceous fusion constructs between a CPAMD protein (e.g., A2M) and a biopharmaceutical moiety (e.g., a therapeutic peptide, polypeptide or protein), where the biopharmaceutical moiety is placed into the RBD domain at a position between the residues that correspond to residues 1402 and 1403 of native human A2M, without removing any of residues of the CPAMD protein. Linker sequences may be used to connect the N-terminus of the biopharmaceutical moiety with the carboxyl end of residue 1402 (SEQ ID NO: 78) and to connect the C-terminus of the biopharmaceutical moiety with the amino end of residue 1403 (SEQ ID NO: 79). An example of a ciRBD fusion construct incorporating the EgA1 nanobody (SEQ ID NO: 27) into A2M is given in SEQ ID NO: 5-6.

iRBD

The term “iRBD” is to be understood as proteinaceous fusion constructs between a CPAMD protein (e.g., A2M) and a biopharmaceutical moiety (e.g., a therapeutic peptide, polypeptide or protein), where the biopharmaceutical moiety replaces the residues of the RBD domain corresponding to the residues spanning from and including position 1392, to and including 1403 in native human A2M. The biopharmaceutical moiety is connected to residue 1391 by an N-terminal linker (SEQ ID NO: 80) and to residue 1404 by a C-terminal linker (SEQ ID NO: 81). An example of an iRBD fusion construct incorporating the EgA1 nanobody (SEQ ID NO: 27) into A2M is given in SEQ ID NO: 84-85.

miRBD

The term “miRBD” is to be understood as proteinaceous fusion constructs between a CPAMD protein (e.g., A2M) and a biopharmaceutical moiety (e.g., a therapeutic peptide, polypeptide or protein), where the biopharmaceutical moiety replaces the residues of the RBD domain corresponding to the residues spanning from and including position 1393, to and including 1395 of native human A2M. The biopharmaceutical moiety is connected to residue 1392 by an N-terminal linker (SEQ ID NO: 82) and to residue 1396 by a C-terminal linker (SEQ ID NO: 83). An example of a miRBD fusion construct incorporating the EgA1 nanobody (SEQ ID NO: 27) into A2M is given in SEQ ID NO: 86-87.

tRBD

The term “tRBD” is to be understood as proteinaceous fusion constructs between a CPAMD protein (e.g., A2M) and a biopharmaceutical moiety (e.g., a therapeutic peptide, polypeptide or protein), where the biopharmaceutical moiety is incorporated into a position C-terminal to the RBD domain. Furthermore, residues 1393 to 1402 of the RBD domain, or the corresponding residues of the RBD domain of another CPAMD protein, are modified to enable the formation of an α-helix with a sequence that is complementary to that of another α-helix that is positioned at the N-terminus of the biopharmaceutical moiety. The RBD domain α-helix and the α-helix at the N-terminus of the biopharmaceutical moiety are designed to interact with each with coiled-coil interactions. These coiled-coil interactions bring the biopharmaceutical moiety into a position relative to the RBD domain which facilitates shielding of the biopharmaceutical moiety by the CPAMD protein (e.g., A2M). The biopharmaceutical moiety is connected at its N-terminus to the C-terminus of its adjacent a-helix by a 2-residue linker, and the α-helix itself is connected at its N-terminus to the C-terminus of the RBD domain by a 15-residue linker. An example of a tRBD fusion construct incorporating the EgA1 nanobody (SEQ ID NO: 27) into A2M is given in SEQ ID NO: 92-93.

### Epitope

In the present context, the term “epitope” refers to the part of an antigen that is recognized by the immune system.

### Eukaryotic Expression Vector

In the present context, the term “eukaryotic expression vector” refers to a tool used to introduce a specific coding polynucleotide sequence into a target cell, comprising expression control sequences (e.g., a suitable promoter sequence) operatively linked to a nucleotide sequence to be expressed.

### Sequence Identity

In the present context, the term “sequence identity” is here defined as the sequence identity between genes or proteins at the nucleotide, base or amino acid level, respectively. Specifically, a DNA and an RNA sequence are considered identical if the transcript of the DNA sequence can be transcribed to the corresponding RNA sequence.

Thus, in the present context, “sequence identity” is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned.

To determine the percent identity of two amino acid sequences or of two nucleic acids, the sequences are aligned for optimal comparison purposes (e.g., gaps may be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide at the corresponding position in the second sequence, then the molecules are identical in that position. The percent identity between the two sequences is a function of the number of identical positions shared by the sequences (i.e., % identity=#of identical positions/total #of positions (e.g., overlapping positions)×100). In one embodiment, the two sequences are the same length.

In another embodiment, the two sequences are of different length and gaps are seen as different positions. One may manually align the sequences and count the number of identical amino acids. Alternatively, alignment of two sequences for the determination of percent identity may be accomplished using a mathematical algorithm. Such an algorithm is incorporated into the BLASTN and BLASTX programs of (Altschul et al. 1990). BLAST nucleotide searches may be performed with the NBLAST program, to obtain nucleotide sequences homologous to a nucleic acid molecule of the invention. BLAST protein searches may be performed with the BLASTX program, to obtain amino acid sequences homologous to a protein molecule of the invention.

To obtain gapped alignments for comparison purposes, Gapped BLAST may be utilized. Alternatively, PSI-Blast may be used to perform an iterated search that detects distant relationships between molecules. When utilizing the BLASTN, BLASTX, and Gapped BLAST programs, the default parameters of the respective programs may be used. See http://www.ncbi.nlm.nih.gov. Alternatively, sequence identity may be calculated after the sequences have been aligned e.g. by the BLAST program in the EMBL database (www.ncbi.nlm.gov/cgi-bin/BLAST). Generally, the default settings with respect to e.g. “scoring matrix” and “gap penalty” may be used for alignment. In the context of the present invention, the BLASTN and PSI BLAST default settings may be advantageous.

The percent identity between two sequences may be determined using techniques similar to those described above, with or without allowing gaps. In calculating percent identity, only exact matches are counted. An embodiment of the present invention thus relates to sequences of the present invention that has some degree of sequence variation.

### Subject

The term “subject” comprises humans of all ages, other primates (e.g., cynomolgus monkeys, rhesus monkeys); mammals in general, including commercially relevant mammals, such as cattle, pigs, horses, sheep, goats, mink, ferrets, hamsters, cats and dogs, as well as birds. Preferred subjects are humans.

The term “subject” also includes healthy subjects of the population and, in particular, healthy subjects, who are exposed to pathogens and in need of protection against infection, such as health personnel.

It should be noted that embodiments and features described in the context of one of the aspects of the present invention also apply to the other aspects of the invention.

All patent and non-patent references cited in the present application, are hereby incorporated by reference in their entirety.

### DETAILED DESCRIPTION OF THE INVENTION

### CPAMD Proteins

The proteinaceous prodrug construct described herein comprise a complement 3- and pregnancy zone protein-like, alpha-2-macroglobulin domain-containing (CPAMD) protein, or a variant or fragment thereof. The CPAMD protein, or the variant or fragment thereof, comprises a bait region with at least one protease cleavage site and a Receptor Binding Domain (RBD).

In some embodiments, the one or more drugs are positioned inside or in the vicinity of any one of loops 1-4 of the RBD (e.g., loop 1, loop 2, loop 3, or loop 4). In one specific embodiment, the one or more drugs are positioned inside loop 2. In another specific embodiment, the one or more drugs are positioned inside loop 4. In a further specific embodiment, the one or more drugs is positioned in the vicinity of loop 2 of the RBD.

In a proteinaceous prodrug construct of the invention, the CPAMD protein, or the variant or fragment thereof, shields the one or more drugs. The CPAMD protein, or the variant or fragment thereof, is capable of altering conformation upon proteolytic cleavage of the at least one protease cleavage site, making the one or more drugs accessible.

While the invention is described in more detail in reference to proteinaceous prodrug constructs in which the CPAMD protein is an alpha-2-macroglobulin (A2M), or a variant or functional homolog thereof, a person of skill in the art of proteinaceous prodrug design will appreciate that other CPAMD proteins can take the place of A2M.

In some embodiments, the proteinaceous prodrug construct is a fusion protein. In one embodiment, the proteinaceous fusion construct, comprises a member of the CPAMD family fused to one or more drugs; or a modified member of the CPAMD family (2) fused to one or more drugs; wherein the one or more drugs are positioned inside or in the vicinity of the RBD domain of A2M. Proteinaceous fusion construct and proteinaceous prodrug are used interchangeably herein.

In some embodiments, the one or more drugs are inserted in any one of loops 1-4 of the RBD. In some embodiments, the loop is modified by addition, substitution, or deletion of one or more amino acids to accommodate the one or more drugs. In some embodiments, the one or more drugs replace one or more amino acids of the loop. In some embodiments, the loop is loop 2 of the RDB. In some embodiments, the loop is loop 4 of the RBD.

As discussed herein, placing one or more drugs in the vicinity of loop 2 of the RBD can be accomplished by insertion of the one or more drugs inside or within 5 amino acid residues of loop 2 (e.g., by replacing one or more residues, or by direct insertion). Similarly, this can be accomplished by insertion of the one or more drugs inside or within 5 amino acid residues of loop 1, loop 3, or loop 4 (e.g., by replacing one or more residues, or by direct insertion). Loops 1, 3 and 4 have respective distances of 27 Å, 21 Å, and 25 Å to loop 2, as calculated from their centers of mass. In the ciRBD fusion approach described herein, the shortest restraint between the drug and loop 2 is the 15-residue C-terminal linker. From an average length of 3.5 Å per amino acid residue, it can be calculated that the one or more drugs can be positioned about 52 Å (e.g., about 50 Å, about 40 Å, about 30 Å, or about 20 Å) away from loop 2 and occupy a position where its accessibility is dependent on the conformation of the CPAMD protein (e.g., A2M).

As an alternative to direct fusion, approaches can be designed to place the drug within an equivalent distance to loop 2 and with a similar orientation relative to the RBD domain as achieved by the direct fusion approach, through other means. For example, as described herein, coiled coil interactions or high-affinity interactions can be used to anchor a drug to loop 2 (e.g., as in the tRBD approach described herein).

Table 1 provides an illustrative list of CPAMD proteins that may be used to implement the invention and also indicated the positions and sequence of each loop with the CPAMD protein.

TABLE 1
Positions of loops 1-4 in various CPAMD proteins

| | NCBI or | | | |
|---|---|---|---|---|
| CPAMD | UniProtKB/Swiss-Prot | | | |
| protein | reference sequence | RBD | Position | Sequence |
| CPAMD1 | NP_000055.2 | Loop 1 | 1395- | DQDATMS |
| (a.k.a. C3) | (SEQ ID NO: 134) | | 1401 | (SEQ ID NO: 145) |
| | | Loop 2 | 1414- | DTDDLKQLANGVDRYI |
| | | | 1429 | (SEQ ID NO: 146) |
| | | Loop 3 | 1449- | DKVSNQT |
| | | | 1455 | (SEQ ID NO: 147) |
| | | Loop 4 | 1480- | YAYYNLEE |
| | | | 1487 | (SEQ ID NO: 148) |
| CPAMD2 | NP_009224.2 | Loop 1 | 1473- | WRNGKVGLSGMA |
| (a.k.a. C4A) | (SEQ ID NO: 135) | | 1484 | (SEQ ID NO: 149) |
| | | Loop 2 | 1497- | LRADLEKLTSLSDRYV |
| | | | 1512 | (SEQ ID NO: 150) |
| | | Loop 3 | 1527- | DSVPTSR |
| | | | 1533 | (SEQ ID NO: 151) |
| | | Loop 4 | 1557- | YDYYNPER |
| | | | 1564 | (SEQ ID NO: 152) |
| CPAMD3 | NP_001002029.3 | Loop 1 | 1473- | WRNGKVGLSGMA |
| (a.k.a. C4B) | (SEQ ID NO: 136) | | 1484 | (SEQ ID NO: 153) |
| | | Loop 2 | 1497- | LRADLEKLTSLSDRYV |
| | | | 1512 | (SEQ ID NO: 154) |
| | | Loop 3 | 1527- | DSVPTSR |
| | | | 1533 | (SEQ ID NO: 155) |
| | | Loop 4 | 1557- | YDYYNPER |
| | | | 1564 | (SEQ ID NO: 156) |
| CPAMD4 | NP_001726.2 | Loop 1 | 1411- | SREESSSGSSHA |
| (a.k.a. C5) | (SEQ ID NO: 137) | | 1422 | (SEQ ID NO: 157) |
| | | Loop 2 | 1435- | NEEDLKALVEGVDQLF |
| | | | 1450 | (SEQ ID NO: 158) |
| | | Loop 3 | 1465- | NSIPSSD |
| | | | 1471 | (SEQ ID NO: 159) |
| | | Loop 4 | 1496- | YEYHRPDK |
| | | | 1503 | (SEQ ID NO: 160) |
| CPAMD5 | NP_000005.3 | Loop 1 | 1368- | SYTGSRSASNMA |
| (a.k.a. A2M) | (SEQ ID NO: 138) | | 1379 | (SEQ ID NO: 161) |
| | | Loop 2 | 1392- | LKPTVKMLERSNHV |
| | | | 1405 | (SEQ ID NO: 162) |
| | | Loop 3 | 1420- | DKVSNQT |
| | | | 1426 | (SEQ ID NO: 163) |
| | | Loop 4 | 1450- | YDYYETDE |
| | | | 1457 | (SEQ ID NO: 164) |
| CPAMD6 | NP_002855.2 | Loop 1 | 1374- | SYTGNRPASNMV |
| (a.k.a. PZP) | (SEQ ID NO: 139) | | 1385 | (SEQ ID NO: 165) |
| | | Loop 2 | 1398- | LKPTVKMLERSSSV |
| | | | 1411 | (SEQ ID NO: 166) |
| | | Loop 3 | 1426- | EQVTNQT |
| | | | 1432 | (SEQ ID NO: 167) |
| | | Loop 4 | 1456- | YDYYETDE |
| | | | 1463 | (SEQ ID NO: 168) |
| CPAMD7 | NP_598000.2 | Loop 1 | 1304- | SFSGPGRSGMA |
| (a.k.a. CD109) | (SEQ ID NO: 140) | | 1314 | (SEQ ID NO: 169) |
| | | Loop 2 | 1325- | MVPSEAISLSETV |
| | | | 1337 | (SEQ ID NO: 170) |
| | | Loop 3 | 1352- | DSVNETQ |
| | | | 1358 | (SEQ ID NO: 171) |
| | | Loop 4 | 1382- | VDYYEPRR |
| | | | 1389 | (SEQ ID NO: 172) |
| CPAMD8 | NP_056507.3 | Loop 1 | 1562- | RWLHAGSSNMA |
| | (SEQ ID NO: 141) | | 1572 | (SEQ ID NO: 173) |
| | | Loop 2 | 1585- | DIESLEQLLLDKHMGM |
| | | | 1600 | (SEQ ID NO: 174) |
| | | Loop 3 | 1615- | DEIPSRC |
| | | | 1621 | (SEQ ID NO: 175) |
| | | Loop 4 | 1646- | YDYYEPAF |
| | | | 1653 | (SEQ ID NO: 176) |
| CPAMD9 | NP_653271.3 | Loop 1 | 1354- | SYVGSRSSSNMA |
| (a.k.a. A2ML1) | (SEQ ID NO: 142) | | 1365 | (SEQ ID NO: 177) |
| | | Loop 2 | 1378- | MEGTNQLLLQQPLV |
| | | | 1391 | (SEQ ID NO: 178) |
| | | Loop 3 | 1406- | DELIKNT |
| | | | 1412 | (SEQ ID NO: 179) |
| | | Loop 4 | 1436- | YDYYLPDE |
| | | | 1443 | (SEQ ID NO: 180) |
| Ovostatin 1 | Q6IE37.2 | Loop 1 | 1098- | RYTGIRNKSSMV |
| | (SEQ ID NO: 143) | | 1109 | (SEQ ID NO: 181) |
| | | Loop 2 | 1122- | TMSSIEEVNNRSLI |
| | | | 1135 | (SEQ ID NO: 182) |
| | | Loop 3 | 1144- | EYKRA |
| | | | 1148 | (SEQ ID NO: 183) |
| | | Loop 4 | 1172- | YDYYEKGR |
| | | | 1179 | (SEQ ID NO: 184) |
| Ovostatin 2 | Q6IE36.2 | Loop 1 | 1336- | KYTGIRNKSSMV |
| | (SEQ ID NO: 144) | | 1347 | (SEQ ID NO: 185) |
| | | Loop 2 | 1360- | TMSSIEELENKGQV |
| | | | 1373 | (SEQ ID NO: 186) |
| | | Loop 3 | 1388- | EYKRA |
| | | | 1394 | (SEQ ID NO: 187) |
| | | Loop 4 | 1418- | YDYYEKGR |
| | | | 1425 | (SEQ ID NO: 188) |

In some embodiments, the CPAMD protein is selected from the group consisting of C3, C4A, C4B, C5, PZP, A2ML1, CD109, CPAMD8, Ovostatin homologue 1, Ovostatin homologue 2, and A2M. In some embodiments, the CPAMD protein is selected from A2M, PZP, Ovostatin 1, and Ovostatin 2, and functional homologs thereof. In some embodiments, the CPAMD protein is human A2M, or a functional homolog thereof, e.g., a mammalian A2M. In a particular embodiment, the CPAMD protein is A2M.

In one embodiment, the CPAMD protein is a human CPAMD protein, such as one of the proteins listed in Table 1, or a variant thereof. In some embodiments, the human CPAMD protein is a variant that has been modified as described herein, e.g., the variant may comprise a modified bait region.

In some embodiments, the CPAMD protein has at least about 70% sequence identity to at least one of the full-length CPAMD protein sequences listed in Table 1. In some embodiments, the CPAMD protein has at least about 75% sequence identity to at least one of the full-length CPAMD protein sequences listed in Table 1. In one embodiment, the CPAMD protein has at least about 80% sequence identity to at least one of the full-length CPAMD protein sequences listed in Table 1. In one embodiment, the CPAMD protein has at least about 85% sequence identity to at least one of the full-length CPAMD protein sequences listed in Table 1. In one embodiment, the CPAMD protein has at least about 90% sequence identity to at least one of the full-length CPAMD protein sequences listed in Table 1.

In one embodiment, the CPAMD protein has at least about 91% sequence identity to at least one of the full-length CPAMD protein sequences listed in Table 1. In one embodiment, the CPAMD protein has at least about 92% sequence identity to at least one of the full-length CPAMD protein sequences listed in Table 1. In one embodiment, the CPAMD protein has at least about 93% sequence identity to at least one of the full-length CPAMD protein sequences listed in Table 1. In one embodiment, the CPAMD protein has at least about 94% sequence identity to at least one of the full-length CPAMD protein sequences listed in Table 1. In one embodiment, the CPAMD protein has at least 95% sequence identity to at least one of the full-length CPAMD protein sequences listed in Table 1. In one embodiment, the CPAMD protein has at least about 96% sequence identity to at least one of the full-length CPAMD protein sequences listed in Table 1. In one embodiment, the CPAMD protein has at least about 97% sequence identity to at least one of the full-length CPAMD protein sequences listed in Table 1. In one embodiment, the CPAMD protein has at least about 98% sequence identity to at least one of the full-length CPAMD protein sequences listed in Table 1. In one embodiment, the CPAMD protein has at least about 99% sequence identity to at least one of the full-length CPAMD protein sequences listed in Table 1.

In further embodiments, the CPAMD protein is a human CPAMD protein, such as the proteins listed in Table 1, with the proviso that
a. the bait region is modified as described herein; and/or
b. one or more drugs (e.g., a therapeutic peptide, polypeptide or protein) are inserted into the RBD region, e.g., into loop 2, such as by removing one or more of the residues of loop 2 as described above.

In another embodiment, the CPAMD protein has at least about 70% sequence identity to at least one of the full-length CPAMD protein sequences listed in Table 1, with the proviso that
a. the bait region is modified as described herein; and/or
b. one or more drugs (e.g., a therapeutic peptide, polypeptide or protein) are inserted into the RBD region, e.g., into loop 2, such as by removing one or more of the residues of loop 2 as described above.

In one embodiment, the CPAMD protein has at least about 80% sequence identity to at least one of the full-length CPAMD protein sequences listed in Table 1, with the proviso that
a. the bait region is modified as described herein; and/or
b. one or more drugs (e.g., a therapeutic peptide, polypeptide or protein) are inserted into the RBD region, e.g., into loop 2, such as by removing one or more of the residues of loop 2 as described above.

In one embodiment, the CPAMD protein has at least about 85% sequence identity to at least one of the full-length CPAMD protein sequences listed in Table 1, with the proviso that
a. the bait region is modified as described herein; and/or
b. one or more drugs (e.g., a therapeutic peptide, polypeptide or protein) are inserted into the RBD region, e.g., into loop 2, such as by removing one or more of the residues of loop 2 as described above.

In one embodiment, the CPAMD protein has at least about 90% (e.g., at least 91%, at least 92%, at least 93% or at least 95%) sequence identity to at least one of the full-length CPAMD protein sequences listed in Table 1, with the proviso that
a. the bait region is modified as described herein; and/or
b. one or more drugs (e.g., a therapeutic peptide, polypeptide or protein) are inserted into the RBD region, e.g., into loop 2, such as by removing one or more of the residues of loop 2 as described above.

In one embodiment, the CPAMD protein has at least about 95% (e.g., at least 96%, at least 97%, at least 98% or about 99%) sequence identity to at least one of the full-length CPAMD protein sequences listed in Table 1, with the proviso that
a. the bait region is modified as described herein; and/or
b. one or more drugs (e.g., a therapeutic peptide, polypeptide or protein) are inserted into the RBD region, e.g., into loop 2, such as by removing one or more of the residues of loop 2 as described above.

The RBD domains and bait regions of the CPAMD proteins listed in Table 1 are described in Table 2. The RBD domains (also referred to as the “MG8 domain”) and bait regions (also referred to as “anaphylactic domain” in some CPAMD proteins) were identified on the basis of their functional equivalence to the corresponding domain/region of human A2M.

TABLE 2
Positions of the RBD and bait regions in various CPAMD proteins

| Protein name | Feature | Position | Sequence |
|---|---|---|---|
| CPAMD1 | RBD domain | 1352-1495 | AKDQLTCNKFDLKVTIKPAPETEKR |
| (a.k.a. C3) | | | PQDAKNTMILEICTRYRGDQDATM |
| | | | SILDISMMTGFAPDTDDLKQLANG |
| | | | VDRYISKYELDKAFSDRNTLIIYLD |
| | | | KVSHSEDDCLAFKVHQYFNVELIQP |
| | | | GAVKVYAYYNLEESCTRFYHP |
| | | | (SEQ ID NO: 189) |
| | Bait region | 672-760 | SVOLTEKRMDKVGKYPKELRKCCE |
| | | | DGMRENPMRFSCQRRTRFISLGEAC |
| | | | KKVFLDCCNYITELRRQHARASHL |
| | | | GLARSNLDEDIIAEEN (SEQ ID NO: |
| | | | 190) |
| CPAMD2 | RBD domain | 1388-1572 | DMKNTTCQDLQIEVTVKGHVEYT |
| (a.k.a. C4A) | | | MEANEDYEDYEYDELPAKDDPDAP |
| | | | LQPVTPLQLFEGRRNRRRREAPKV |
| | | | VEEQESRVHYTVCIWRNGKVGLSG |
| | | | MAIADVTLLSGFHALRADLEKLTSL |
| | | | SDRYVSHFETEGPHVLLYFDSVPTS |
| | | | RECVGFEAVQEVPVGLVQPASATL |
| | | | YDYYNPERRCSVFYGA (SEQ ID |
| | | | NO: 191) |
| | Bait region | 680-771 | NVNFQKAINEKLGQYASPTAKRCC |
| | | | QDGVTRLPMMRSCEQRAARVQQP |
| | | | DCREPFLSCCQFAESLRKKSRDKGQ |
| | | | AGLQRALEILQEEDLIDEDD (SEQ |
| | | | ID NO: 192) |
| CPAMD3 | RBD domain | 1388-1572 | DMKNTTCQDLQIEVTVKGHVEYT |
| (a.k.a. C4B) | | | MEANEDYEDYEYDELPAKDDPDAP |
| | | | LQPVTPLQLFEGRRNRRRREAPKV |
| | | | VEEQESRVHYTVCIWRNGKVGLSG |
| | | | MAIADVTLLSGFHALRADLEKLTSL |
| | | | SDRYVSHFETEGPHVLLYFDSVPTS |
| | | | RECVGFEAVQEVPVGLVQPASATL |
| | | | YDYYNPERRCSVFYGA (SEQ ID |
| | | | NO: 193) |
| | Bait region | 680-771 | NVNFQKAINEKLGQYASPTAKRCC |
| | | | QDGVTRLPMMRSCEQRAARVQQP |
| | | | DCREPFLSCCQFAESLRKKSRDKGQ |
| | | | AGLQRALEILQEEDLIDEDD (SEQ |
| | | | ID NO: 194) |
| CPAMD4 | RBD domain | 1369-1511 | STSEEVCSFYLKIDTQDIEASHYRG |
| (a.k.a. C5) | | | YGNSDYKRIVACASYKPSREESSSG |
| | | | SSHAVMDISLPTGISANEEDLKALV |
| | | | EGVDQLFTDYQIKDGHVILQLNSIP |
| | | | SSDFLCVRFRIFELFEVGFLSPATFT |
| | | | VYEYHRPDKQCTMFYST (SEQ ID |
| | | | NO: 195) |
| | Bait region | 678-762 | TLQKKIEEIAAKYKHSVVKKCCYD |
| | | | GACVNNDETCEQRAARISLGPRCIK |
| | | | AFTECCVVASQLRANISHKDMQLG |
| | | | RLHMKTLLPVSK (SEQ ID NO: 196) |
| CPAMD5 | RBD domain | 1335-1474 | EKEEFPFALGVQTLPQTCDEPKAHT |
| (a.k.a. A2M) | | | SFQISLSVSYTGSRSASNMAIVDVK |
| | | | MVSGFIPLKPTVKMLERSNHVSRTE |
| | | | VSSNHVLIYLDKVSNQTLSLFFTVL |
| | | | QDVPVRDLKPAIVKVYDYYETDEF |
| | | | AIAEYNAPCSKDLGNA (SEQ ID NO: |
| | | | 197) |
| | Bait region | 690-728 | PQLQQYEMHGPEGLRVGFYESDV |
| | | | MGRGHARLVHVEEPHT (SEQ ID |
| | | | NO: 198) |
| CPAMD6 | RBD domain | 1341-1482 | EKEDSPFALKVQTVPQTCDGHKAH |
| (a.k.a. PZP) | | | TSFQISLTISYTGNRPASNMVIVDVK |
| | | | MVSGFIPLKPTVKMLERSSSVSRTE |
| | | | VSNNHVLIYVEQVTNQTLSFSFMVL |
| | | | QDIPVGDLKPAIVKVYDYYETDESV |
| | | | VAEYIAPCSTDTEHGNV (SEQ ID |
| | | | NO: 199) |
| | Bait region | 686-734 | SVIPSVSAGAVGQGYYGAGLGVVE |
| | | | RPYVPQLGTYNVIPLNNEQSSGPVP |
| | | | (SEQ ID NO: 200) |
| CPAMD7 | RBD domain | 1274-1420 | SIQNQEAFDLDVAVKENKDDLNHV |
| (a.k.a. CD109) | | | DLNVCTSFSGPGRSGMALMEVNLL |
| | | | SGFMVPSEAISLSETVKKVEYDHGK |
| | | | LNLYLDSVNETQFCVNIPAVRNFKV |
| | | | SNTQDASVSIVDYYEPRRQAVRSY |
| | | | NSEVKLSSCDLCSDVQGCRPCEDG |
| | | | A (SEQ ID NO: 201) |
| | Bait region | 653-683 | GVYDNAEYAERFMEENEGHIVDIH |
| | | | DFSLGSS (SEQ ID NO: 202) |
| CPAMD8 | RBD domain | 1500-1670 | PVAKPAFQLLVSLQEPEAQGRPPPM |
| | | | PASAAEGSRGDWPPADDDDPAAD |
| | | | QHHQEYKVMLEVCTRWLHAGSSN |
| | | | MAVLEVPLLSGFRADIESLEQLLLD |
| | | | KHMGMKRYEVAGRRVLFYFDEIPS |
| | | | RCLTCVRFRALRECVVGRTSALPVS |
| | | | VYDYYEPAFEATRFYNVSTHSPLA |
| | | | RE (SEQ ID NO: 203) |
| | Bait region | 709-740 | GGLYTDEAVPAFQPHTGSLVAVAP |
| | | | SRHPPRTE (SEQ ID NO: 204) |
| CPAMD9 | RBD domain | 1321-1454 | PTNMKTFSLSVEIGKARCEQPTSPR |
| (a.k.a. A2ML1) | | | SLTLTIHTSYVGSRSSSNMAIVEVK |
| | | | MLSGFSPMEGTNQLLLQQPLVKKV |
| | | | EFGTDTLNIYLDELIKNTQTYTFTIS |
| | | | QSVLVTNLKPATIKVYDYYLPDEQ |
| | | | ATIQYSDPCE (SEQ ID NO: 205) |
| | Bait region | 695-727 | SHRSPEYSTAMGAGGGHPEAFESST |
| | | | PLHQAEDS (SEQ ID NO: 206) |
| Ovostatin 1* | RBD domain | 1096-1185 | IFRYTGIRNKSSMVVIDVKMLSGFT |
| | | | PTMSSIEEVNNRSLIFQHKDSYIEYK |
| | | | RADSFPFSVEQSNLVFNIQPAPAMV |
| | | | YDYYEKGRQATAMP (SEQ ID NO: |
| | | | 207) |
| | Bait region | 616-617 | II (SEQ ID NO: 208) |
| Ovostatin 2 | RBD domain | 1308-1432 | PKKASGFSLSLEIVKNYSLTVFDLT |
| | | | VNLKYTGIRNKSSMVVIDVKMLSG |
| | | | FTPTMSSIEELENKGQVMKTEVKN |
| | | | DHVLFYLENVFGRADSFTFSVEQSN |
| | | | LVFNIQPAPGMVYDYYEKDGEAFL |
| | | | LTN (SEQ ID NO: 209) |
| | Bait region | 673-716 | VMERRLPLPKPLYLETENYGPMHS |
| | | | VPSRIACRGENADYVEQAII (SEQ ID |
| | | | NO: 210) |

*The available Ovostatin1 sequence (Q6IE37.2) is of poor quality and likely incomplete.

When one or more drugs (e.g., a therapeutic peptide, polypeptide or protein) are introduced into the RBD, they are sterically hindered from interacting with other proteins such as their therapeutics targets. The RBD domain is itself a small domain (˜16 kDa). Without wishing to be bound by any particular theory, the inventors believe that it is unlikely that the RBD domain is able to sterically hinder the one or more drugs (e.g., a therapeutic peptide, polypeptide or protein) on its own, especially considering that linkers are typically present between the one or more drugs and the RBD domain. Without wishing to be bound by any particular theory, the inventors therefore believe that other portions or multiple copies of the CPAMD protein contribute to the surrounding and sequestering of the one or more drugs. For example, naturally occurring CPAMD proteins (e.g., A2M) form homotetramers.

In some embodiments, CPAMD protein (e.g., A2M) forms a multimer (e.g., a dimer or tetramer). In some embodiments, the multimer comprises identical subunits (e.g., homodimers or homotetramers). Without wishing to be bound by any particular theory, the inventors believe it to be possible that contributions from one or more adjacent subunits contribute to the sequestering of the one or more drugs.

Two human CPAMD proteins are known to form dimers (typically stabilized by one or more disulfide bridges), namely A2M and pregnancy zone protein (PZP, a.k.a. CPAMD6). In A2M, the disulfide-bridged dimer participates in additional non-covalent interactions with another disulfide-bridged dimer, primarily through their LNK regions, to form a tetramer. This tetramer formation is also seen in ovostatins, such as those that have been characterized in ducks, chickens, and frogs. The two human ovostatins, ovostatin 1 and ovostatin 2 are also predicted to be tetramers.

Accordingly, in some embodiments, a proteinaceous prodrug construct in accordance with the invention is capable of forming a multimer, e.g., a dimer or a tetramer. In some embodiments, the multimer is a heteromultimer (e.g., a heterodimer or heterotetramer). More typically, the multimer is a homodimer or homotetramer.

In some embodiments, the multimer (e.g., dimer or tetramer) formation occurs via a LNK region of the CPAMD protein. In some embodiments, a tetramer is formed by two disulfide-bridged dimers (e.g., two homodimers).

The cysteines which form the inter-subunit disulfide bonds that are responsible for the disulfide-bridged dimer are found in two loops, one of which is located on the MG3 domain of the CPAMD protein and one of which is located on the MG4 domain of the CPAMD protein. These loops are defined in Table 3. The LNK region that has been shown to participate in interactions between the two disulfide-bridged dimers in tetramer-forming CPAMD proteins is also defined in Table 3.

TABLE 3
Regions for multimer formation in various CPAMD proteins.

| Protein name | Feature name | Position | Sequence |
|---|---|---|---|
| CPAMD5 (a.k.a. | MG3 loop with | 271- | YSDASDCHGEDSQA |
| A2M) | cysteine | 285 | (SEQ ID NO: 211) |
| | MG4 loop with | 424- | NYKDRSPCYGYQWVSEEHEEA |
| | cysteine | 444 | (SEQ ID NO: 212) |
| | LNK loop | 644- | NRHNVYINGITYTPVSSTNEKD |
| | | 665 | (SEQ ID NO: 213) |
| CPAMD6 (a.k.a. | MG3 loop with | 271- | LSRVLNCDKQE |
| PZP) | cysteine | 281 | (SEQ ID NO: 214) |
| | MG4 loop with | 420- | FTVHPNLCFHYSWVAEDHQGA |
| | cysteine | 440 | (SEQ ID NO: 215) |
| | LNK loop | 642- | RPFFIHNGAIYVPLSSNEAD |
| | | 661 | (SEQ ID NO: 216) |
| Ovostatin 1 | MG3 loop with | 246- | YFSSSNCEKNENE |
| | cysteine | 258 | (SEQ ID NO: 217) |
| | MG4 loop with | 375- | RHQRTEECYLPSWLTPQYLDA |
| | cysteine | 395 | (SEQ ID NO: 218) |
| | LNK loop | 579- | PQRDMFYNGLYYTPVSNYGDGD |
| | | 600 | (SEQ ID NO: 219) |
| Ovostatin 2 | MG3 loop with | 251- | YFSSSNCEKNENE |
| | cysteine | 263 | (SEQ ID NO: 220) |
| | MG4 loop with | 403- | TYVRPKSCYLPSWLTPQYLDA |
| | cysteine | 423 | (SEQ ID NO: 221) |
| | LNK loop | 627- | PQRDMFYNGLYYTPVSNYGDGD |
| | | 648 | (SEQ ID NO: 222) |

iRBD, miRBD, ciRBD, and tRBD as described herein create proteinaceous prodrug constructs by “locking” the location of a drug (e.g., a peptide, polypeptide or protein) in the vicinity of loop 2 (residues 1392-1405) on the RBD of CPAMD protein (e.g., A2M), either by direct fusion in the iRBD/miRBD/ciRBD approaches or by anchoring of the drug to this location with coiled-coil interactions in the tRBD approach. Other approaches that are able to anchor the drug in this general location relative to the RBD domain will be apparent to the skilled person.

In some embodiments, the proteinaceous prodrug construct comprises a first interaction domain and the one or more drugs comprise a second interaction domain, wherein the first and second interaction domains form a complex positioning the one or more drugs in the vicinity of any one of loops 1-4 (e.g., loop 1, loop 2, loop 3, or loop 4) of the RBD. In one specific embodiment, the first and second interaction domains form a complex positioning the one or more drugs in the vicinity of loop 2. In another specific embodiment, the first and second interaction domains form a complex positioning the one or more drugs in the vicinity of loop 4. In some embodiments, the first interaction domain and the second interaction domain form a coiled coil structure.

Without wishing to be bound by any particular theory, the inventors believe that the use of first and second interaction domains to position the one or more drugs in the vicinity of loop 2 of the RBD allows the CPAMD protein to take on its “native” conformation, thereby sequestering the one or more drugs inside it (thus, shielding it from interactions with one or more targets). Spatial proximity may be achieved, e.g., by inserting the first interaction domain in loop 2 of the RBD, or in one of loops 1-3 (e.g., loop 4) of the RBD.

One approach is the “docking” of a drug into the CPAMD protein (e.g., A2M). This can be done using a molecule that has an inherent affinity for loop 2 of the RBD, such as a functional fragment of the LRP1 receptor or an antibody (e.g., a nanobody) which recognizes a loop 2 epitope.

Alternatively, the RBD of the CPAMD protein (e.g., A2M) could be modified to facilitate such docking. For example, a tag sequence could be introduced into the RBD (e.g., in the “ciRBD” position), and the drug could be fused to an antibody (e.g., a nanobody or similar small binding domain) which recognizes the tag.

Accordingly, in some embodiments, the first interaction domain is a tag or epitope sequence within loop 2 of the RBD and the second interaction domain is a functional fragment of a receptor or antibody that is capable of binding specifically to the tag or epitope sequence.

### A2M

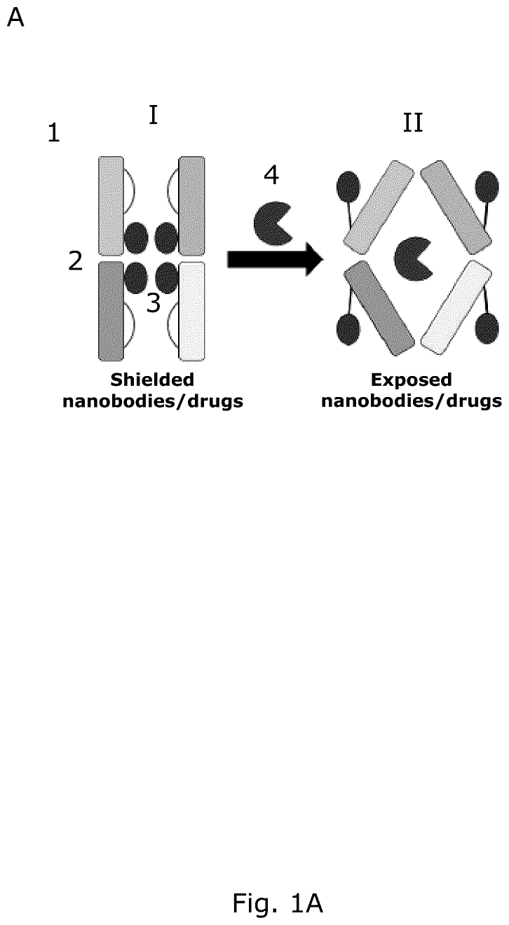

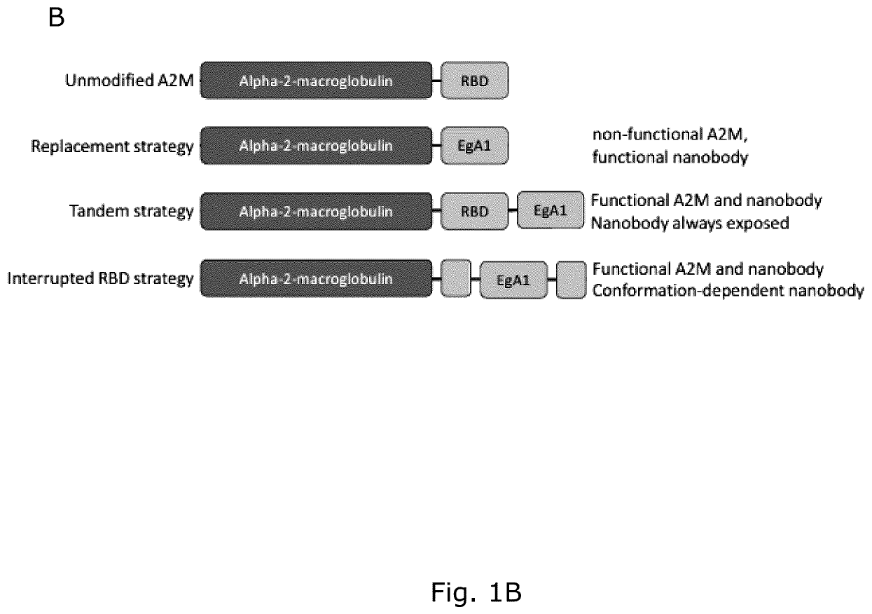

Alpha-2-macroglobulin (A2M) is a protein found at high concentrations (normally 1-5 g/L) in human plasma. A2M is a protease inhibitor with a well-characterized mechanism of action. First, proteases cleave an exposed and vulnerable stretch of sequence called the bait region, which is permissive to cleavage by most human proteases. Bait region cleavage triggers a conformational change in A2M that causes A2M to collapse around the protease, trapping the protease within A2M and preventing it from accessing additional large protein substrates (FIG. 1). Up to two proteases can be inhibited by a single A2M protein if cleavage is rapid and sequential. In addition to the trapping of the instigating protease(s), there are two additional consequences of the triggered conformational change: (i) a cryptic binding site on A2M for the LRP1 receptor is exposed, resulting in the binding of A2M-protease complex by cell surface LRP1 and the rapid clearance of these complexes, e.g. from circulation, by LRP1-expressing hepatocytes, and (ii) a reactive thiol ester moiety is exposed on A2M, allowing the formation of covalent bonds to the trapped protease.

The present invention describes the incorporation of biopharmaceutical moieties into A2M in such a manner that the binding ability of the biopharmaceutical moiety is regulated by the conformation of A2M. Biopharmaceutical moieties suitable for use with the present invention include therapeutic peptides, polypeptides or proteins such as antibodies (e.g., single-chain or single domain antibodies such as scFvs and nanobodies). In the native conformation of A2M, the incorporated biopharmaceutical moiety occupies a shielded position where it has a decreased ability to interact with its therapeutic target. After the conformation of A2M is altered by proteolytic cleavage of the bait region (or, alternatively, by aminolysis of the thiol ester of A2M using methylamine, which triggers a similar conformational change), the biopharmaceutical moiety demonstrates an increased ability to interact with its target. By modification of A2M's bait region sequence, specific proteases can be designated as able to cleave the bait region and trigger this conformational change. Altogether, this can be used to produce proteinaceous fusion constructs of A2M and a biopharmaceutical moiety (e.g., a therapeutic peptide, polypeptide or protein) that function as protease-activated prodrug versions of the biopharmaceutical moiety.

In one embodiment, the invention provides a proteinaceous prodrug construct, comprising: (a) an alpha-2-macroglobulin (A2M) protein, or a variant or fragment thereof, and (b) one or more drugs, wherein (i) the A2M protein, or the variant or fragment thereof comprises (1) a bait region with at least one protease cleavage site, and (2) a Receptor Binding Domain (RBD), (ii) the one or more drugs are positioned inside or in the vicinity of the RBD, and (iii) the A2M protein, or the variant or fragment thereof, shields the one or more drugs and is capable of altering conformation upon proteolytic cleavage of the at least one protease cleavage site, making the one or more drugs accessible.

In some embodiments, the present invention relates to a proteinaceous fusion construct comprising alpha-2-macroglobulin (A2M), fused to one or more drugs; or a modified A2M fused to one or more drugs; wherein the one or more drugs are positioned inside or in the vicinity of the RBD domain of A2M.

In some embodiments, the one or more drugs is positioned inside or in the vicinity of any one of loops 1-4 of the RBD. In some embodiments, the proteinaceous prodrug construct is a fusion protein. In some embodiments, the one or more drugs are positioned inside any one of loops 1-4 of the RBD. In some embodiments, the loop is loop 1. In some embodiments, the loop is loop 2. In some embodiments, the loop is loop 3. In some embodiments, the loop is loop 4. In some embodiments, the loop is modified, in relation to a wildtype loop sequence, by addition, substitution or deletion of one or more amino acids to accommodate the one or more drugs. In some embodiments, the one or more drugs replace one or more amino acids of the loop.

In one embodiment, the one or more drugs, is inaccessible when the bait region in alpha-2-macroglobulin (A2M) has not been proteolytically cleaved; and the one or more drugs, is accessible when the bait region in alpha-2-macroglobulin (A2M) has been proteolytically cleaved.

In one embodiment, the cleavage of the bait region can be effectuated by serine-, cysteine-, aspartic- and/or metalloproteinases.

The drug can be positioned on different locations within the sequence of the proteinaceous fusion construct.

The skilled person will be able to recognize the parts of the proteinaceous fusion construct, which originates from A2M. Thus, in embodiments where a drug is inserted into the sequence of A2M, the resulting fusion construct can be seen as a first part of A2M, a drug, and a second part of A2M. In such cases, the skilled person will be able to recognize the first- and the second part of A2M as a complete molecule. Thus, in a particular embodiment, sequence identity of A2M is to be calculated from two separate parts, based on the sequence deriving from A2M, and thus not including the one or more drugs.

In one embodiment, the A2M molecule is a mammalian A2M molecule or variant thereof, such as a human A2M molecule.

In one embodiment, the A2M molecule is a human A2M molecule, such as the sequence according to SEQ ID NO: 1, or a variant thereof. In some embodiments, the human A2M molecule is a variant that has been modified as described herein, e.g., the variant may comprise a modified bait region.

In some embodiments, the A2M molecule has at least about 70% sequence identity to the sequence according to SEQ ID NO: 1. In some embodiments, the A2M molecule has at least about 75% sequence identity to the sequence according to SEQ ID NO: 1. In one embodiment, the A2M molecule has at least about 80% sequence identity to the sequence according to SEQ ID NO: 1. In one embodiment, the A2M molecule has at least about 85% sequence identity to the sequence according to SEQ ID NO: 1. In one embodiment, the A2M molecule has at least about 90% sequence identity to the sequence according to SEQ ID NO: 1.

In one embodiment, the A2M molecule has at least about 91% sequence identity to the sequence according to SEQ ID NO: 1. In one embodiment, the A2M molecule has at least about 92% sequence identity to the sequence according to SEQ ID NO: 1. In one embodiment, the A2M molecule has at least about 93% sequence identity to the sequence according to SEQ ID NO: 1. In one embodiment, the A2M molecule has at least about 94% sequence identity to the sequence according to SEQ ID NO: 1. In one embodiment, the A2M molecule has at least 95% sequence identity to the sequence according to SEQ ID NO: 1. In one embodiment, the A2M molecule has at least about 96% sequence identity to the sequence according to SEQ ID NO: 1. In one embodiment, the A2M molecule has at least about 97% sequence identity to the sequence according to SEQ ID NO: 1. In one embodiment, the A2M molecule has at least about 98% sequence identity to the sequence according to SEQ ID NO: 1. In one embodiment, the A2M molecule has at least about 99% sequence identity to the sequence according to SEQ ID NO: 1.

In further embodiments, the A2M molecule is a human A2M molecule, such as the sequence according to SEQ ID NO: 1, with the proviso that
c. the bait region is modified as described above; and/or
d. one or more drugs (e.g., a therapeutic peptide, polypeptide or protein) are inserted into the RBD region, e.g., into loop 2, such as by removing one or more of the residues of loop 2 as described above.

In another embodiment, the A2M molecule has at least about 70% sequence identity to the sequence according to SEQ ID NO: 1, with the proviso that
c. the bait region is modified as described above; and/or
d. one or more drugs (e.g., a therapeutic peptide, polypeptide or protein) are inserted into the RBD region, e.g., into loop 2, such as by removing one or more of the residues of loop 2 as described above.

In one embodiment, the A2M molecule has at least about 80% sequence identity to the sequence according to SEQ ID NO: 1, with the proviso that
c. the bait region is modified as described above; and/or
d. one or more drugs (e.g., a therapeutic peptide, polypeptide or protein) are inserted into the RBD region, e.g., into loop 2, such as by removing one or more of the residues of loop 2 as described above.

In one embodiment, the A2M molecule has at least about 85% sequence identity to the sequence according to SEQ ID NO: 1, with the proviso that
c. the bait region is modified as described above; and/or
d. one or more drugs (e.g., a therapeutic peptide, polypeptide or protein) are inserted into the RBD region, e.g., into loop 2, such as by removing one or more of the residues of loop 2 as described above.

In one embodiment, the A2M molecule has at least about 90% (e.g., at least 91%, at least 92%, at least 93% or at least 95%) sequence identity to the sequence according to SEQ ID NO: 1, with the proviso that
c. the bait region is modified as described above; and/or
d. one or more drugs (e.g., a therapeutic peptide, polypeptide or protein) are inserted into the RBD region, e.g., into loop 2, such as by removing one or more of the residues of loop 2 as described above.

In one embodiment, the A2M molecule has at least about 95% (e.g., at least 96%, at least 97%, at least 98% or about 99%) sequence identity to the sequence according to SEQ ID NO: 1, with the proviso that
c. the bait region is modified as described above; and/or
d. one or more drugs (e.g., a therapeutic peptide, polypeptide or protein) are inserted into the RBD region, e.g., into loop 2, such as by removing one or more of the residues of loop 2 as described above.

In one embodiment, the one or more drugs is positioned between 1391 and 1405 in SEQ ID NO: 1.

In another embodiment, the one or more drugs is positioned after position 1335 in SEQ ID NO: 1.

In another embodiment, the one or more drugs is positioned before position 1474 in SEQ ID NO: 1.

In another embodiment, the one or more drugs are positioned between 1391 and 1405 in SEQ ID NO: 1 or after position 1335 but before position 1474 in A2M.

In another embodiment, the A2M molecule comprises one or more of the mutations K1393A, K1397A, T654C, and/or T661C.

K1393A and K1397A remove A2M's interactions with the receptors LRP1 and Grp78, respectively. LRP1 mediates clearance of cleaved A2M, Grp78 induces mitogenic signaling in cells when bound. Both of these receptor interactions are potentially problematic in a drug, as such it can be beneficial to remove these amino acids.

The T654C and T661C mutations introduce a disulfide which bridges the two disulfide-dimers of A2M, so that the entire A2M tetramer becomes stabilized by disulfide bonds. This prevents the splitting of A2M into its two halves, which can occur during physiological conditions such as inflammation (due to oxidative damage to A2M).

In an aspect of the invention, the invention relates to a proteinaceous fusion construct comprising alpha-2-macroglobulin (A2M), comprising a bait region with at least one protease cleavage site, said A2M being fused to a peptide drug positioned within residues 1392-1404, 1368-1379, or 1420-1426, of the Receptor Binding Domain (RBD) of A2M. In particular, in such an aspect it may occur that the peptide drug is inaccessible when the bait region in A2M has not been proteolytically cleaved; and the peptide drug, is accessible when the bait region in A2M has been proteolytically cleaved.

While the foregoing paragraphs describe the positioning of the one or more drugs within the RBD domain and the introduction of disulfide bridges in reference to A2M, a person of skill in the art of proteinaceous prodrug design will appreciate that other CPAMD proteins can take the place of A2M and can identify corresponding residues in these CPAMD proteins to implement the invention (e.g., using the residue numbers provided in Tables 1 and 2 as a guide).

### The Drug

The proteinaceous prodrug construct can comprise one or more drugs or biopharmaceutical moieties (e.g., a therapeutic peptide, polypeptide or protein).

In one embodiment, said one or more drugs is selected from the group consisting of: an antigen-targeting moiety (e.g., an antibody or an antibody mimetics), a cytokine, the extracellular region of a cell surface receptor, the extracellular region of a cell surface ligand, and a receptor agonist.

In another embodiment, said one or more drugs is selected from the group consisting of: toxins, enzymes, and protein conjugates with small molecule drugs analogous to ADCs. For example, the one or more drugs may contain appropriate sites for small molecule conjugation, for example cysteine residues.

In a further embodiment, said toxin(s) is selected from bacterially derived anthrax and diphtheria toxins.

In yet another embodiment, said one or more drugs is an antigen-targeting moiety, such as a single-chain variable fragment of antibody.

In one embodiment, said antigen-targeting moiety is selected from the group consisting of: antibody, nanobody, diabody, and single-chain variable fragment. In some embodiments, the antigen-targeting moiety is a single-chain or single-domain antibody. In particular embodiments, the antigen-targeting moiety is a single-chain variable fragment.

In another embodiment, said antigen-targeting moiety is selected from the group consisting of a monoclonal antibody, a recombinant antibody, a single chain antibody, a bispecific antibody, a nanobody, an antibody wherein the heavy chain and the light chain are connected by a flexible linker, an Fv molecule, an antigen binding fragment, a Fab fragment, a Fab′ fragment, a F(ab′)₂ molecule, a fully human antibody, a humanized antibody, and a chimeric antibody or a fragment or derivative thereof.

In some embodiments, the antigen-targeting moiety specifically binds to an antigen as an antagonist (e.g., the antigen-targeting moiety is capable of inhibiting the binding of a ligand to its receptor). In some embodiments, the antigen-targeting moiety specifically binds to an antigen as an agonist (e.g., the antigen-targeting moiety is capable of inducing signaling by binding to a receptor).

In some embodiments, the antigen-targeting moiety specifically binds to an antigen selected from the group consisting of BTLA, OX40, LAG3, NRP1, VEGF, HER2, CEA, CD19, CD20, Amyloid beta, HER3, IGF-1R, MUC1, EpCAM, CD22, VEGFR-2, PSMA, GM-CSF, CXCR4, CD30, CD70, FGFR2, BCMA, CD44, ICAM-1, Notch1, MHC, CD28, IL-1R1, TCR, Notch3, FGFR3, TGF-β, TGFBR1, TGFBR2, CD109, GITR, CD47, Alpha-synuclein, CD26, LRP1, CD52, IL-4Rα, VAP-1, EPO Receptor, Integrin av, TIM-3, Grp78, LIGHT, TLR2, TLR3, PAR-2, NRP2, GLP-1 receptor, Hedgehog, and Syndecan 1.

In one embodiment, the one or more drugs has a size of at the most 100 kDa, such as at the most 85 kDa, such as at the most 75 kDa, such as at the most 65 kDa, such as at the most 55 kDa, such as at the most 50 kDa, such as at the most 40 kDa, such as at the most 30 kDa, such as at least 10 kDa.

In one embodiment, the one or more drugs comprise at most 900 amino acids, such as at most 770 amino acids, such as at most 680 amino acids, such as at most 590 amino acids, such as at most 500 amino acids, such as at most 450 amino acids, such as at most 360 amino acids, such as the most 270 amino acids, such as at least 90 amino acids.

In one embodiment, the antigen targeting moiety is selected from the group consisting of anti-PD1, anti-PD-L1, anti-EGFR, anti-CTLA4, anti-CD137, anti-CD3, and anti-TNFα.

In one embodiment, the antigen targeting moiety is selected from the group consisting of Atezolizumab, EgA1, Ipilimumab, Nivolumab, KN035, Urelumab, Foralumab, Muromonab, and Adalimumab, or a therapeutically active scFv, fragment or variant thereof comprising one or more CDRs, all three heavy chain CDRs, all three light chain CDRs, all three heavy chain and all three light chain CDRs, a heavy chain variable region, and/or a light chain variable region of any of the foregoing antigen targeting moieties.

In some embodiments, the one or more drugs are selected from the group consisting of ANB032, rosnilimab, LY3361237, Encelimab, Cobolimab, Imsidolimab, Dostarlimab, or a therapeutically active scFv, fragment or variant thereof comprising one or more CDRs, all three heavy chain CDRs, all three light chain CDRs, all three heavy chain and all three light chain CDRs, a heavy chain variable region, and/or a light chain variable region of any of the foregoing antigen targeting moieties.

As outlined above, cytokines may be used as the drug in the present invention. Cytokines are described as a category of small proteins that induce cell signaling.

In one embodiment, said one or more drugs is a cytokine selected from the group consisting of chemokines, interferons, interleukins, lymphokines and tumor necrosis factors.

In another embodiment, said one or more drugs is a cytokine selected from the group consisting of IL1, IL1alpha, IL1beta, IL2, IL3, IL4, IL6, IL7, IL8, IL9, IL10, IL11, IL12, IL13, IL14, IL15, IL16, IL17, 118, IL19, IL20, IL21, IL22, IL23, IL24, IL25, IL26, IL27, IL28, IL29, IL30, IL31, IL32, IL33, IL34, IL35 and IL36.

In a further embodiment, said one or more drugs is a cytokine selected from the group consisting of IL2, IFN-α, IL-15, IL-21, IL-10, IL-12, IL-17, GM-CSF, TGF-β, CSF-1, insulin, GLP-1, HGH, VEGF, PDGF, BMP, EPO, G-CSF, IL-11, IFN-γ, and IFN-β.

In the preferred embodiment, said one or more drugs is IL2. IL 2 is tested in example 9.

In one embodiment, the antigen-targeting moiety is encoded by an amino acid sequence selected from the group consisting of SEQ ID NO: 27-43. In another embodiment, the antigen-targeting moiety has or comprises an amino acid sequence selected from the group consisting of SEQ ID NO: 27-43. In a further embodiment, the antigen-targeting moiety has an amino acid sequence having at least about 80% sequence identity, such as at least about 85%, 90%, or even about 95% sequence identity, to a sequence selected from the group consisting of SEQ ID NO: 27-43. If variance is introduced into the antigen-targeting moiety, it is preferred that the CDR sequences are not modified.

In another embodiment, a nucleic acid sequence encoding an antigen-targeting moiety is selected from the group consisting of: SEQ ID NO: 6, 8, 10, 12, 14, 16, 18, 20, 22, 24, and 26 or a fragment thereof having at least about 90% sequence identity to any of SEQ ID NO: 6, 8, 10, 12, 14, 16, 18, 20, 22, 24, and 26: particularly about 95% identity to SEQ ID NO: 6, 8, 10, 12, 14, 16, 18, 20, 22, 24, and 26. In another embodiment, the amino acid sequence is encoded by a nucleic acid sequence selected from the group consisting of: SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, and SEQ ID NO: 26 or a fragment thereof having at least about 90% sequence identity to any of SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, and SEQ ID NO: 26: particularly about 95% identity to SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, and SEQ ID NO: 26.

In a further embodiment, the proteinaceous prodrug construct according to invention comprises 1-5 drugs, such as 1-4, such as 1-3, such as 1-2. In a specific embodiment, the proteinaceous prodrug construct according to invention comprises 1 drug.

### The Bait Region

As previously described, the proteinaceous prodrug construct's unique protease-trapping mechanism of inhibition (FIG. 1) is initiated when a protease cleaves within the exposed and highly susceptible bait region.

In some embodiment, a proteinaceous prodrug construct in accordance with the invention comprises a CPAMD protein (e.g., A2M) with a modified bait region. In some embodiments, the bait region is modified to change the selection of proteases that are able to cleave it and trigger the conformational change of the CPAMD protein (e.g., A2M). For example, the bait region may be modified to be cleaved by a particular protease or class of proteases (e.g., MMPs such as MMP2).

The modification enables a construction of a CPAMD protein (e.g., A2M), comprising a bait region with no protease cleavage sites. The modification does not affect the structure and function of the CPAMD protein (e.g., A2M), but facilitates that proteases are not able to stimulate the conformational change in the CPAMD protein as seen in wild-type CPAMD proteins (e.g., A2M). A bait region that cannot be cleaved by proteases is referred to herein as a “tabula rasa bait region”. An a CPAMD protein (e.g., A2M) comprising a bait region that cannot be cleaved by proteases is referred to herein as a “tabula rasa” bait region.

In particular embodiments, at least one protease cleavage site is introduced into the tabula rasa bait region. By using such a modified bait region, it is possible to control which proteases are able to cleave and thereby introduce the conformational change to the proteinaceous prodrug construct.

For example, for the tabula rasa bait region to prevent cleavage by proteases, it may comprise an engineered amino acid sequence that is flexible and/or hydrophilic. In some embodiments, the engineered amino acid sequence comprises a sequence of glycine, serine, alanine, threonine, and/or proline residues. In some embodiments, the engineered amino acid sequence replaces all or a portion of a wildtype bait region. In some embodiments, the engineered amino acid sequence is about 15-51 amino acids, such as about 30-40, such as about 31-39, such as about 32-35. In a particular embodiment, the length of the engineered amino acid sequence is about 32-33 amino acids. In some embodiments, the engineered amino acid sequence replaces all of the wildtype bait region and has a length equivalent to the wildtype bait region.

In other embodiments, for the tabula rasa bait region to prevent cleavage by proteases, it is composed of a series of amino acid repeats. The series of amino acid repeats may replace part or all of the native bait region. Thus, in one embodiment, said tabula rasa bait region comprises a series of amino acid repeats. An example of a series of three amino acid repeats are Gly-Gly-Ser, Gly-Gly-Gly, Gly-Ser-Gly, Gly-Ser-Ser, Ser-Gly-Gly, Ser-Gly-Ser, Ser-Ser-Gly, Ser-Ser-Ser.

Each series of three amino acids are either repeated or combined with each other.

Thus, in one embodiment, tabula rasa bait region is comprised of one or more amino acid repeats, wherein the repeats are selected from the list consisting of Gly-Gly-Ser, Gly-Gly-Gly, Gly-Ser-Gly, Gly-Ser-Ser, Ser-Gly-Gly, Ser-Gly-Ser, Ser-Ser-Gly and Ser-Ser-Ser.

In another embodiment, the proteinaceous prodrug construct, comprises a tabula rasa bait region comprised of one or more amino acid repeats, wherein the repeats are amino acid triplets comprised by Ser, Gly, and Ala residues.

In a further embodiment, tabula rasa bait region is comprised of one or more amino acid repeats, wherein the repeats are selected from the list consisting of Gly-Gly-Ser, Gly-Gly-Gly, Gly-Ser-Gly, Gly-Ser-Ser, Ser-Gly-Gly, Ser-Gly-Ser, Ser-Ser-Gly, Ser-Ser-Ser and Ala.

In another embodiment, the proteinaceous prodrug construct, comprises a tabula rasa bait region comprised of one or more amino acid repeats, wherein the repeats are selected from the list consisting of Gly-Gly-Ser, Gly-Gly-Gly, Gly-Gly-Ala, Gly-Ser-Gly, Gly-Ser-Ser, Gly-Ser-Ala, Gly-Ala-Ser, Gly-Ala-Gly, Gly-Ala-Ala, Ser-Gly-Gly, Ser-Gly-Ser, Ser-Gly-Ala, Ser-Ser-Gly, Ser-Ser-Ser, Ser-Ser-Ala, Ser-Ala-Gly, Ser-Ala-Ser, Ser-Ala-Ala, Ala-Gly-Ser, Ala-Gly-Gly, Ala-Gly-Ala, Ala-Ser-Gly, Ala-Ser-Ser, Ala-Ser-Ala, Ala-Ala-Ser, Ala-Ala-Gly and Ala-Ala-Ala.

In one embodiment, the bait region comprises 5, such as 7, such as 9, such as 11, such as 13, such as 15, such as 17 repeats. In one particular embodiment, the bait region comprises 13 repeats.

In another embodiment, the bait region comprises about 5-17 repeats, such as about 7-15, such as about 9-13.

The total length of the bait region can vary between 15 and 51 amino acids.

Thus, in one embodiment, the length of the tabula rasa bait region is about 15-51 amino acids, such as about 30-40, such as about 31-39, such as about 32-35. In a particular embodiment, the length of the tabula rasa bait region is about 32-33 amino acids.

A specific embodiment of the tabula rasa bait region, consisting of 13 Gly-Gly-Ser repeats, can be seen in SEQ ID NO: 124. Thus, in one embodiment the tabula rasa bait region is SEQ ID NO: 124.

### Cleavage Sites

For the proteinaceous prodrug construct to be effective as a medicament, and to control the activity of the proteinaceous prodrug construct, individual protease cleavage sites can be introduced into the tabula rasa bait region. Thus, the skilled person can control which proteases that are able to cleave and thereby introduce the conformational change to the proteinaceous prodrug construct.

The invention is not limited to introducing a single protease cleavage site. In some embodiments, the bait region can have a number of cleavage sites, which are cleaved by different proteases.

Thus, in one embodiment said, bait region comprises one or more protease cleavage sites (e.g., two, three or four protease cleavage sites).

In another embodiment, said bait region comprises only one protease cleavage site.

In some embodiments, said bait region comprises only one protease cleavage site which can be cleaved by a protease selected from the group consisting of activated protein C, ADAM10, ADAM12, ADAM15, ADAM17/TACE, ADAM9, ADAMDEC1, ADAMTS1, ADAMTS4, ADAMTS5, BACE, BMP-1, Caspase 1, Caspase 10, Caspase 14, Caspase 2, Caspase 3, Caspase 4, Caspase 5, Caspase 6, Caspase 7, Caspase 8, Caspase 9, Cathepsin A, Cathepsin B, Cathepsin C, Cathepsin D, Cathepsin E, Cathepsin G, Cathepsin K, Cathepsin L, Cathepsin S, Cathepsin V/L2, Cathepsin X/Z/P, Chymase, Cruzipain, DESC1, DPP-4, Elastase, FAP, Granzyme B, Guanidinobenzoatase, Hepsin, HtrA1, Neutrophil Elastase, KLK10, KLK11, KLK13, KLK14, KLK4, KLK5, KLK6, KLK7, KLK8, Lactoferrin, Legumain, Marapsin, Matriptase-2, Meprin, MMP1, MMP8, MMP10, MMP11, MMP12, MMP13, MMP14, MMP15, MMP16, MMP17, MMP19, MMP2, MMP20, MMP23, MMP24, MMP26, MMP27, MMP3, MMP7, MMP8, MMP9, MT-SP1/Matriptase, Neprilysin, NS3/4A, Otubain-2, PACE4, Plasmin, PSA, PSMA, Renin, Thrombin, TMPRSS2, TMPRSS3, TMPRSS4, tPA, Tryptase, uPA, ADAM8, FVIIa, FIXa, Furin, Fxa, FXIa, FXIIa, and TAFI.

In a further embodiment, said bait region contains a single cleavable site selected from the group of SEQ ID NO: 96-123.

In some embodiments, said bait region contains only one single protease cleavage site which can be cleaved by a matrix metalloprotease (MMP). In a particular embodiment, said bait region contains only one single protease cleavage site which can be cleaved by a protease selected from the group consisting of MMP2, MMP9, MMP14, MMP1, MMP3, MMP13, MMP17, MMP11, MMP8, MMP10, and MMP19.

The bait region may also comprise two cleavage sites. Thus, in one embodiment said bait region comprises two protease cleavage sites.

In another embodiment, said bait region comprises exactly two cleavable sites, one of which is cleavable by the group of proteases consisting of MMP2, MMP9, MMP14, MMP1, MMP3, MMP13, MMP17, MMP11, MMP8, MMP10, and MMP19, and the other of which is cleavable by the group of proteases consisting of activated protein C, ADAM10, ADAM12, ADAM15, ADAM17/TACE, ADAM9, ADAMDEC1, ADAMTS1, ADAMTS4, ADAMTS5, BACE, BMP-1, Caspase 1, Caspase 10, Caspase 14, Caspase 2, Caspase 3, Caspase 4, Caspase 5, Caspase 6, Caspase 7, Caspase 8, Caspase 9, Cathepsin A, Cathepsin B, Cathepsin C, Cathepsin D, Cathepsin E, Cathepsin G, Cathepsin K, Cathepsin L, Cathepsin S, Cathepsin V/L2, Cathepsin X/Z/P, Chymase, Cruzipain, DESC1, DPP-4, Elastase, FAP, Granzyme B, Guanidinobenzoatase, Hepsin, HtrA1, Neutrophil Elastase, KLK10, KLK11, KLK13, KLK14, KLK4, KLK5, KLK6, KLK7, KLK8, Lactoferrin, Legumain, Marapsin, Matriptase-2, Meprin, MT-SP1/Matriptase, Neprilysin, NS3/4A, Otubain-2, PACE4, Plasmin, PSA, PSMA, Renin, Thrombin, TMPRSS2, TMPRSS3, TMPRSS4, tPA, Tryptase, uPA, ADAM8, FVIIa, FIXa, Furin, Fxa, FXIa, FXIIa and TAFI.

In another embodiment, said bait region comprises exactly two cleavable sites selected from the group of SEQ ID NO: 96-123.

In a further embodiment, the bait region is free from protease cleavage sites recognized by human proteases except MMPs. In some embodiments, said bait region contains one or more (e.g., at least two or three) protease cleavage sites which can be cleaved by one or more (e.g., at least two or three) MMPs.

In yet a further embodiment, the bait region is free from protease cleavage sites recognized by human proteases except for a single cleavage site.

As seen by the examples, the bait region can be highly modified, and the skilled person will be able to select any suitable cleavage site into the bait region, according to the needed specificity. Accordingly, in particular embodiments, a proteinaceous prodrug construct in accordance with the invention comprises a CPAMD protein (e.g., A2M) comprising a modified bait region that can be selectively cleaved by one or more proteases.

A protease site is “selectively cleavable” when cleavage occurs only or predominantly in the presence of one particular protease. A modified bait region may be engineered to comprise one or more (e.g., at least two or three) cleavage sites, wherein each of the cleavage sites is “selectively cleavable” by a different protease. For example, a modified bait region may be engineered to comprise one or two or three unique recognition sites, each specific for a different protease.

Exemplary MMP cleavage sites include the A21A, B74, C9 and S1. In a specific embodiment, the bait region comprises one or more (e.g., at least two or three) of the A21A, B74, C9 and/or the S1 cleavage sites. Exemplary modified bait regions comprising said cleavage sites can be seen in SEQ ID NO: 126-133. In another specific embodiment, the bait region comprises a lysine, such as in SEQ ID NO: 125.

In some embodiments, the modified bait region comprises an engineered amino acid sequence that is flexible and/or hydrophilic. In some embodiments, the engineered amino acid sequence comprises a sequence of glycine, serine, alanine, threonine, and/or proline residues. In some embodiments, the engineered amino acid sequence comprises a combination of glycine, serine, and/or alanine residues. In some embodiments, the engineered amino acid sequence replaces a wildtype bait region and has a length equivalent to the wildtype bait region.

In one embodiment, the wildtype bait region has been replaced by a combination of glycine, serine, and/or alanine residues with an equivalent length to the wildtype bait region.

Exemplary sequences, where cleavage sites have been inserted into the tabula rasa region can be seen in any of the sequences identified by SEQ ID NO: 125-133.

In another embodiment, only a part of the wildtype bait region has been replaced by the described tabula rasa region, such as in SEQ ID NO: 130 where the C-terminal quarter of the wildtype bait region is retained.

In another embodiment, one or more cleavage sites in the bait region have been replaced by a combination of glycine, serine, and/or alanine residues.

In one embodiment, the bait region comprises one or more repeats, such as at least 5, such as at least 6, such as at least 7, such as at least 8. In a particular embodiment, the length of the tabula rasa bait region is, at least about 10 repeats.

In one embodiment, the bait region has a size of about 8 kDa, such as at the most about 5 kDa, such as at the most about 4 kDa, such as at the most about 3 kDa, such as at the most about 2 kDa. In a particular embodiment, the bait region has a size of at the most about 2.5 kDa.

In one embodiment, the length of the bait region is about 15 to 51 amino acids. In one embodiment, the length of the bait region is about 30-40 amino acids, e.g., about 31-39 amino acids or 32-35 amino acids. In a particular embodiment, the total length of the bait region is about 32-33 amino acids.

In one embodiment, the bait region comprises an engineered amino acid sequence that is entirely flexible and/or hydrophilic, e.g., a random sequence of glycine, serine, alanine, threonine, and/or proline residues, and optionally one or more protease cleavage sites (e.g., MMP cleavage sites), such that the total length of the bait region, including the repeats and cleavage site(s) is about 15 to 51 amino acids, e.g., about 32-33 amino acids.

In one embodiment, when a protease cleaves the “bait region”, the protease is trapped inside the proteinaceous prodrug construct.

### RBD Domain

As explained above, the prodrug is generated by bringing the drug (e.g., a therapeutic peptide, polypeptide or protein) into contact with the RBD domain, such that the folding of the RBD domain, shields the drug, such that the drug is inaccessible. A therapeutic protein may be brought into contact with the RBD domain by inserting it into the RBD domain or by replacing a part of the RBD domain with the therapeutic protein.

Accordingly, in a typical embodiment of a proteinaceous prodrug construct of the invention, the drug (e.g., a therapeutic peptide, polypeptide or protein) is positioned inside the RBD in such a manner that the CPAMD protein (e.g., A2M) is capable of altering conformation upon proteolytic cleavage of a protease cleavage site comprised within the bait region, thereby making the drug accessible.

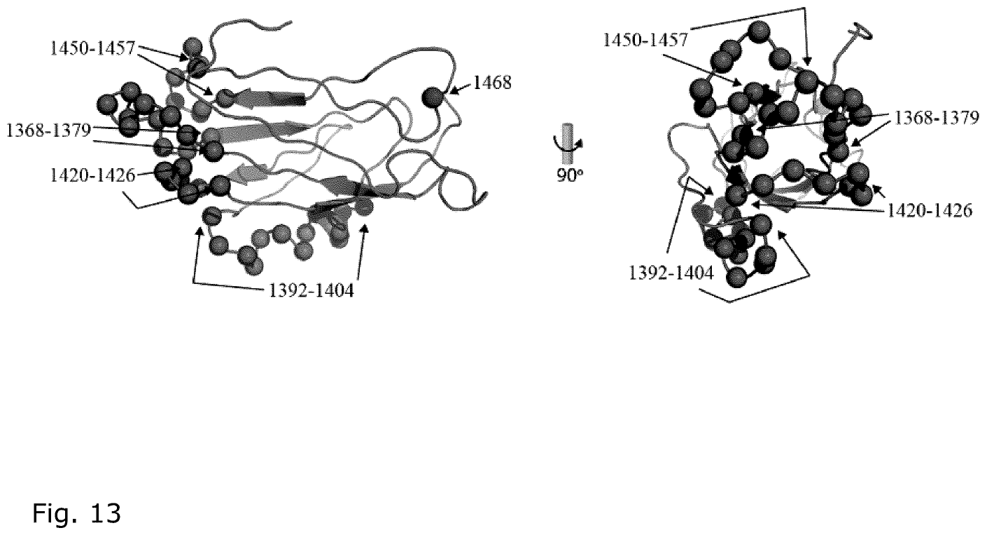

Given the size of the RBD domain, numerous suitable sites exist for insertion into the RBD domain. As visualized by FIG. 13, the RBD domain is largely comprised of beta sheets, and as shown in the examples of the present invention, the loops in between individual beta strands are suitable for insertion of the drug. For example, in the native human A2M protein, loop 1 is formed by amino acid residues 1368-1379, loop 2 by amino acid residues 1392-1404, loop 3 by amino acid residues 1420-1426, and loop 4 by amino acid residues 1450-1457.

In one embodiment, the drug is positioned in the RBD domain of A2M within loop 2 (at a position between residue 1391 and 1405, e.g., between 1392 and 1404, of native human A2M). In one embodiment, the drug is positioned in the RBD domain of A2M by replacing one or more amino acids corresponding to the region formed residues 1391 to 1405, or residues 1392 to 1404, of the native human protein. In one embodiment, the drug is positioned in the RBD domain of A2M between amino acids corresponding to residues 1391 to 1405 (e.g., residues 1392 to 1404) of the native human protein. In another embodiment, one or more of the amino acids corresponding to residues 1391, 1392, 1393, 1394, 1395, 1396, 1397, 1398, 1399, 1400, 1401, 1402, 1403, 1404, and/or 1405 of the native human protein is/are replaced by the drug. In another embodiment, the drug is positioned after one or more of the amino acids corresponding to residues 1391, 1392, 1393, 1394, 1395, 1396, 1397, 1398, 1399, 1400, 1401, 1402, 1403, or 1404 of the native human protein.

In one embodiment, the drug is positioned in the RBD domain of A2M within loop 1 (at a position between residue 1368-1379 of native human A2M). In one embodiment, one or more of the amino acids corresponding to residues 1368, 1369, 1370, 1371, 1372, 1373, 1374, 1375, 1376, 1377,

and/or 1378 of native human A2M is/are replaced by the drug. In another embodiment, the drug is positioned after one or more of the amino acids corresponding to residues 1368, 1369, 1370, 1371, 1372, 1373, 1374, 1375, 1376, 1377, or 1378 of native human A2M.

In one embodiment, the drug is positioned in the RBD domain of A2M in loop 3 (at a position between residue 1420-1426 of native human A2M). In another embodiment, one or more of the amino acids corresponding to residues 1420, 1421, 1422, 1423, and/or 1424 of native human A2M is/are replaced by the drug. In another embodiment, the drug is positioned after one or more of amino acids corresponding to the residues 1420, 1421, 1422, 1423, or 1424 of native human A2M.

In another embodiment, the drug is positioned in the vicinity of the RBD domain of A2M. In one embodiment, the drug is tethered to the C-terminus of A2M's RBD domain and brought into close proximity of residues 1391-1405 of the RBD domain through specific interactions, such as coiled-coil interactions between alpha helices.

While the positioning of the one or more drugs within the RBD domain is described in the foregoing paragraphs is reference to A2M, a person of skill in the art of proteinaceous prodrug design will appreciate that other CPAMD proteins can take the place of A2M and can identify corresponding residues in these CPAMD proteins to implement the invention (e.g., using the residue numbers provided in Table 1 as a guide).

The inventors have found that proteinaceous prodrug constructs in which a drug (e.g., a therapeutic peptide, polypeptide or protein) is positioned within loop 2 or 4 of the RBD domain of a CPAMD protein (e.g., by replacing one or more residues, or by direct insertion) can be expressed successfully at high levels (see, e.g., the proteinaceous fusion constructs referred herein as “ciRBD” and “miRBD”). For example, insertion of the drug between the amino acids corresponding to residues 1402 and 1403 of native human A2M has been found to be particularly advantageous. Replacing the amino acids corresponding to residues 1393-1395 of native human A2M with the drug may be similarly advantageous.

### Linkers

Linkers can be used to insert drugs into the proteinaceous prodrug construct. Any suitable linker may be used. In some embodiments, the linker is a (GGGGS)n (SEQ ID NO: 223) or a (GGS)n. In some embodiments, n=1, 2, 3, 4, 5, or 6.

### Exemplary Proteinaceous Fusion Constructs

In one embodiment, an exemplary proteinaceous fusion construct in accordance with the invention is encoded by an amino acid sequence selected from the group consisting of: SEQ ID NO: 5, SEQ ID NO: 7, SEQ ID NO: 9, SEQ ID NO: 11, SEQ ID NO: 13, SEQ ID NO: 15, and SEQ ID NO: 17, SEQ ID NO: 19, SEQ ID NO: 21, SEQ ID NO: 23, or SEQ ID NO: 25. In one embodiment, the proteinaceous fusion construct is comprised of an amino acid sequence selected from the group consisting of: SEQ ID NO: 5, SEQ ID NO: 7, SEQ ID NO: 9, SEQ ID NO: 11, SEQ ID NO: 13, SEQ ID NO: 15, and SEQ ID NO: 17, SEQ ID NO: 19, SEQ ID NO: 21, SEQ ID NO: 23, and SEQ ID NO: 25.

In another embodiment, the proteinaceous fusion construct has at least about 80% sequence identity, such as at least about 85% sequence identity, about 90% sequence identity, or even about 95% sequence identity, to a sequence selected from the group consisting of: SEQ ID NO: 5, SEQ ID NO: 7, SEQ ID NO: 9, SEQ ID NO: 11, SEQ ID NO: 13, SEQ ID NO: 15, and SEQ ID NO: 17, SEQ ID NO: 19, SEQ ID NO: 21, SEQ ID NO: 23, and SEQ ID NO: 25.

In one embodiment, the amino acid sequence is encoded by a nucleic acid sequence selected from the group consisting of: SEQ ID NO: 6, 8, 10, 12, 14, 16, 18, 20, 22, 24, and 26: or a fragment thereof having at least about 90% sequence identity to anyone of SEQ ID NO: 6, 8, 10, 12, 14, 16, 18, 20, 22, 24, and 26, particularly about 95% identity to anyone of SEQ ID NO: 6, 8, 10, 12, 14, 16, 18, 20, 22, 24, and 26. In one embodiment, the amino acid sequence is encoded by a nucleic acid sequence selected from the group consisting of: SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, and SEQ ID NO: 26: or a fragment thereof having at least SEQ ID NO: about 90% sequence identity to anyone of SEQ ID NO: 6, SEQ ID NO:8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, and SEQ ID NO: 26, particularly about 95% identity to anyone of SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, and SEQ ID NO: 26.

### Exemplary Nucleic Acids

In one aspect, the present invention relates to a nucleic acid encoding a proteinaceous fusion construct according to the invention.

In one embodiment, the nucleic acid according to the invention encodes a proteinaceous fusion construct according to anyone of SEQ ID NO: 5, SEQ ID NO: 7, SEQ ID NO: 9, SEQ ID NO: 11, SEQ ID NO: 13, SEQ ID NO: 15, and SEQ ID NO: 17, SEQ ID NO: 19, SEQ ID NO: 21, SEQ ID NO: 23, or SEQ ID NO: 25.

In another embodiment, the nucleic acid sequence is selected from the group consisting of: SEQ ID NO: 6, 8, 10, 12, 14, 16, 18, 20, 22, 24, and 26: or a fragment thereof having at least about 90% sequence identity to anyone of SEQ ID NO: 6, 8, 10, 12, 14, 16, 18, 20, 22, 24, and 26, particularly about 95% identity to anyone of SEQ ID NO: 6, 8, 10, 12, 14, 16, 18, 20, 22, 24, and 26. In another embodiment, the nucleic acid sequence is selected from the group consisting of: SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, and SEQ ID NO: 26: or a fragment thereof having at least SEQ ID NO: about 90% sequence identity to anyone of SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, and SEQ ID NO: 26, particularly about 95% identity to anyone of SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, and SEQ ID NO: 26.

### Vectors

For a proteinaceous fusion construct to be expressed, the nucleic acid according to the invention is or can be inserted into an expression vector, which is usually a plasmid or a virus designed to control gene expression in a cell. The vector is engineered to contain regulatory sequences that act as enhancers or promotor for an efficient expression of the desired coding sequence carried by the vector. In a non-limiting example, the use of a naked circular plasmid with the key features necessary for expression, including promotor, coding sequence of interest and polyadenylation signal is provided.

Further, to enable an easy production, which might take place using E. coli bacteria, the plasmid comprises a selection marker. This enables production in a bacterium with or without using conventional bacterial resistance selection.

In another embodiment, the present invention relates to a vector comprising the nucleic acid according to the invention.

In one embodiment, the nucleic acid encoding the proteinaceous fusion construct is operatively linked to a promotor and optionally, additionally regulatory sequences that regulate expression of said nucleic acid.

In one embodiment, the vector is a eukaryotic expression vector, particularly a mammalian, e.g., a human expression vector.

In one embodiment, the vector is selected from the group consisting of plasmids, cosmids, phages, bacterial artificial chromosomes (BAC), phagemids, and P1-derived artificial chromosomes.

In one embodiment, the vector is a plasmid.

In one embodiment, said plasmid is selected from the group consisting of TA cloning vectors, Gateway cloning vectors, restriction cloning vectors, Topo cloning vectors, pET vector system, and pBAD vector systems.

### Host Cells

The vector according to the invention is or can be inserted into a host cells for expression of proteinaceous fusion construct according to the invention.

In one aspect, the present invention relates to a host cell comprising a vector according to the invention.

The cells can be either prokaryotic, like bacteria, or eukaryotic cells.

In one embodiment, the host cell is selected from the group consisting of: bacteria and eukaryotes; typically the host cell is a eukaryote.

In another embodiment, the host cell is yeast.

In a typical embodiment, the host cell is a mammalian cell, e.g., a CHO (Chinese Hamster) cell.

In one embodiment, said host cell is human.

In another embodiment, said host cell is the HEK293 cell line or descends from the HEK293 cell line.

### Compositions

A further aspect of the present disclosure relates to a composition, comprising a proteinaceous prodrug construct as described herein. The composition may also comprise a nucleic acid, a vector or a host cell as described herein.

In one embodiment of the present disclosure, the composition comprises a pharmaceutically acceptable carrier. Such a composition can also be referred to as a pharmaceutical composition.

### Therapeutic Uses

The proteinaceous fusion construct according to the invention can be used in the treatment of disease. In further aspects, the composition, a nucleic acid, a vector or a host cell as described herein, can be used in the treatment of disease.

In one aspect, the present invention relates to the proteinaceous prodrug construct, for use in therapy, e.g., as a medicament. In a further aspect, the present invention relates to the composition, a nucleic acid, a vector or a host cell as described herein, for use in therapy, e.g., as a medicament.

In some embodiments, the proteinaceous prodrug construct according to the invention, is for use in treating a disease or disorder of the nervous system, the eye, the circulatory system, the respiratory system, the digestive system, or the skin. In some embodiments, the disease or disorder is a neoplasm, a blood disorder, a metabolic disorder, an autoimmune disease, an immunodeficiency, or an infectious disease. In some embodiments, the neoplasm is a cancer selected from brain cancer, glioblastoma, lung cancer, colorectal cancer, skin cancer, malignant melanoma, pancreas cancer, bladder cancer, liver cancer, breast cancer, eye cancer, and prostate cancer, the cancer is a haematological cancer, such as selected from the group consisting of multiple myeloma, acute myeloblastic leukemia, chronic myelogenic leukemia, acute lymphoblastic leukemia, and chronic lymphocytic leukemia, or the cancer is malignant melanoma, breast cancer, non-small cell lung cancer, pancreatic cancer, head & neck cancer, liver cancer, sarcoma, and B cell lymphoma. In some embodiments, the autoimmune disease is selected from arthritis (e.g., rheumatoid arthritis or psoriatic arthritis), multiple sclerosis, systemic lupus erythematosus, and inflammatory bowel disease.

In one embodiment, the proteinaceous prodrug construct according to the invention, is for use in the treatment of cancer. In another embodiment, the proteinaceous prodrug construct according to the invention, is for use in the treatment of arthritis. In a further embodiment, the composition, a nucleic acid, a vector or a host cell according to the invention, is for use in the treatment of cancer. In yet a further embodiment, the composition, a nucleic acid, a vector or a host cell according to the invention, is for use in the treatment of arthritis.

The proteinaceous prodrug construct, the composition, the nucleic acid, the vector or the host cell as described herein can also be used in methods of treatment. Thus, in another aspect, the disclosure relates to a method of treatment, the method comprising administering a therapeutic amount of the proteinaceous prodrug construct, the composition, the nucleic acid, the vector or the host cell as described herein to a subject in need thereof. The subject in need thereof may be a subject suffering from cancer or arthritis.

In one embodiment, the cancer is a solid tumor. In some embodiments, the cancer is selected from the list consisting of brain cancer, glioblastoma, lung cancer, colorectal cancer, skin cancer, malignant melanoma, pancreas cancer, bladder cancer, liver cancer, breast cancer, eye cancer, and prostate cancer.

In another embodiment, said cancer is a hematological cancer, such as selected from the group consisting of multiple myeloma, acute myeloblastic leukemia, chronic myelogenous leukemia, acute lymphoblastic leukemia, and chronic lymphocytic leukemia.

In a further embodiment, said cancer is malignant melanoma, breast cancer, non-small cell lung cancer, pancreatic cancer, head & neck cancer, liver cancer, sarcoma, or B cell lymphoma.

In some embodiment, a proteinaceous prodrug construct in accordance with the invention comprises a CPAMD protein (e.g., A2M) with a modified bait region. In some embodiments, the bait region is modified to change the selection of proteases that are able to cleave it and trigger the conformational change of the CPAMD protein (e.g., A2M). For example, the bait region is modified to be cleaved by a particular protease or class of proteases (e.g., MMPs such as MMP2).

In one embodiment, the cancer expresses one or more proteases, specific for a cleavage site in the bait region of the CPAMD protein (e.g., A2M). In particular embodiments, a proteinaceous prodrug construct in accordance with the invention comprises a CPAMD protein (e.g., A2M) comprising a modified bait region that can be selectively cleaved by one or more proteases expressed by the cancer.

In one embodiment, the cancer expresses one or more proteases selected from the list consisting of activated protein C, ADAM10, ADAM12, ADAM15, ADAM17/TACE, ADAM9, ADAMDEC1, ADAMTS1, ADAMTS4, ADAMTS5, BACE, BMP-1, Caspase 1, Caspase 10, Caspase 14, Caspase 2, Caspase 3, Caspase 4, Caspase 5, Caspase 6, Caspase 7, Caspase 8, Caspase 9, Cathepsin A, Cathepsin B, Cathepsin C, Cathepsin D, Cathepsin E, Cathepsin G, Cathepsin K, Cathepsin L, Cathepsin S, Cathepsin V/L2, Cathepsin X/Z/P, Chymase, Cruzipain, DESC1, DPP-4, Elastase, FAP, Granzyme B, Guanidinobenzoatase, Hepsin, HtrA1, Neutrophil Elastase, KLK10, KLK11, KLK13, KLK14, KLK4, KLK5, KLK6, KLK7, KLK8, Lactoferrin, Legumain, Marapsin, Matriptase-2, Meprin, MMP1, MMP8, MMP10, MMP11, MMP12, MMP13, MMP14, MMP15, MMP16, MMP17, MMP19, MMP2, MMP20, MMP23, MMP24, MMP26, MMP27, MMP3, MMP7, MMP8, MMP9, MT-SP1/Matriptase, Neprilysin, NS3/4A, Otubain-2, PACE4, Plasmin, PSA, PSMA, Renin, Thrombin, TMPRSS2, TMPRSS3, TMPRSS4, tPA, Tryptase, uPA, ADAM8, FVIIa, FIXa, Furin, FXa, FXIa, FXIIa, and TAFI.

### Subject and Administration

The “subject” as described herein comprises humans of all ages, other primates (e.g., cynomolgus monkeys, rhesus monkeys); mammals in general, including commercially relevant mammals such as cattle, pigs, horses, sheep, goats, mink, ferrets, hamsters, cats, dogs; and/or birds. In a typical embodiment, the subjects are humans.

The term “subject” also includes healthy subjects of the population and, in particular, healthy subjects, who are exposed to pathogens and in need of protection against infection, such as health personnel.

Further, pathogenic infections caused by a virus of the respiratory system can be particularly serious in elderly and weak patients and patients with chronic or congenital dysfunction of the respiratory system, such as asthma, cystic fibrosis, or chronic obstructive pulmonary disease (COPD).

Thus, in an embodiment of the present invention, the subject is selected from the group consisting of; humans of all ages, other primates (e.g., cynomolgus monkeys, rhesus monkeys); mammals in general, including commercially relevant mammals, such as cattle, pigs, horses, sheep, goats, mink, ferrets, hamsters, cats and dogs, as well as birds.

In a particular embodiment, the subject is a human.

### Method for Producing a Proteinaceous Fusion Construct

In one aspect, the present invention relates to a method for producing a proteinaceous fusion construct according to the invention, the method comprising:
introducing into a host cell, an expression vector according to the invention
growing the host cell under conditions that allows for expression of the proteinaceous fusion construct from the vector; and
purifying the proteinaceous fusion construct.

### Numbered Embodiments

The invention is now further described in reference to the following numbered embodiments:

1. A proteinaceous fusion construct comprising alpha-2-macroglobulin (A2M), fused to one or more drugs; or a modified A2M fused to one or more drugs;
wherein the one or more drugs are positioned inside or in the vicinity of the RBD domain of A2M.

2. A proteinaceous fusion construct comprising alpha-2-macroglobulin (A2M), comprising a bait region with at least one protease cleavage site, said A2M being fused to a peptide drug, such as one or more drugs, positioned within residues 1392-1404, 1368-1379, or 1420-1426, of the Receptor Binding Domain (RBD) of A2M.

3. The proteinaceous fusion construct according to embodiment 1 or 2,
a. wherein the one or more drugs, is inaccessible when the bait region in alpha-2-macroglobulin (A2M) has not been proteolytically cleaved; and
b. wherein the one or more drugs, is accessible when the bait region in alpha-2-macroglobulin (A2M) has been proteolytically cleaved.

4. The proteinaceous fusion construct according to anyone of the preceding embodiments, wherein said one or more drugs is selected from the group consisting of: an antigen-targeting moiety, a cytokine, the extracellular region of a cell surface receptor, the extracellular region of a cell surface ligand, and/or a receptor agonist.

5. The proteinaceous fusion construct according to any of embodiment 1 or embodiments 3-4, wherein said bait region comprises one or more protease cleavage sites.

6. The proteinaceous fusion construct according to any of the preceding embodiments, wherein the bait region is free from protease cleavage sites recognized by human proteases except for a single cleavage site.

7. The proteinaceous fusion construct according to any of the preceding embodiments, wherein one or more cleavage sites in the bait region have been replaced by a combination of glycine, serine, and/or alanine residues.

8. The proteinaceous fusion construct according to any of embodiment 1 or embodiments 3-7, wherein the drug is positioned inside the RBD domain of A2M at a position between residue 1335 and 1474, or wherein the drug is positioned in the spatial vicinity of the RBD domain of A2M.

9. The proteinaceous fusion construct according to any of the preceding embodiments, wherein the proteinaceous fusion construct is encoded by an amino acid sequence, or is an amino acid sequence, selected from the group consisting of: SEQ ID NO: 5, SEQ ID NO: 7, SEQ ID NO: 9, SEQ ID NO: 11, SEQ ID NO: 13, SEQ ID NO: 15, and SEQ ID NO: 17, SEQ ID NO: 19, SEQ ID NO: 21, SEQ ID NO: 23, or SEQ ID NO: 25.

10. The proteinaceous fusion construct according to anyone of the preceding embodiments, wherein the A2M molecule is a mammalian A2M molecule or variant thereof, such as a human A2M molecule.

11. A nucleic acid encoding a proteinaceous fusion construct according to any of the preceding embodiments.

12. A vector comprising the nucleic acid according to embodiment 10.

13. A vector according to embodiment 12, wherein the nucleic acid encoding the proteinaceous fusion construct is operatively linked to a promotor and optionally, additionally regulatory sequences that regulate expression of said nucleic acid.

14. A host cell comprising a vector according to any of embodiments 12-13, preferably, wherein the host cell is selected from the group consisting of: bacteria and eukaryote.

15. The proteinaceous fusion construct according to any of embodiment 1-10, for use as a medicament.

16. A method for producing the proteinaceous fusion construct according to anyone of the preceding embodiments, the method comprising:
introducing into the host cell according to embodiment 14 or into any suitable host cell, the expression vector according to anyone of embodiments 12-13;
growing the host cell under conditions that allows for expression of the proteinaceous fusion construct from the vector; and
purifying the proteinaceous fusion construct.

### EQUIVALENTS

Although methods and materials similar or equivalent to those described herein can be used in the practice or testing of the present invention, suitable methods and materials are described below. All publications, patent applications, patents, and other references mentioned herein are incorporated by reference in their entirety. The references cited herein are not admitted to be prior art to the claimed invention. In addition, the materials, methods, and examples are illustrative only and are not intended to be limiting.

### EXAMPLES

The invention will now be described in further detail in the following non-limiting examples.

### Example 1—Overview of the Invention

This shows the overall design and mechanism of the invention, which relates to a technology for producing protease-activated prodrug versions of biopharmaceuticals. With reference to FIG. 1A, a proteinaceous prodrug construct (1) comprises a CPAMD protein, e.g., human alpha-2-macroglobulin (A2M) (2), fused to one or more drugs (3) in such a manner that the drugs accessibility is dependent on the conformational state of the CPAMD protein (e.g., A2M) (2). The CPAMD protein (e.g., A2M) (2) is transformed from an initial “native” conformation to an “activated” conformation by one or more proteases (4). The drugs (3) may be genetically fused to the CPAMD protein (e.g., A2M) (2) at a position where it is inaccessible to its therapeutic target in the “native” conformation (I) of the CPAMD protein (e.g., A2M) (2), but is accessible in the CPAMD protein's (e.g., A2M's) (2) “activated” conformation (II). In this way, the activity of the one or more drugs (3) is spatially restricted to tissues where one or more proteases (4) that can activate the CPAMD protein (e.g., A2M) (2) are present and proteolysis-competent. As the CPAMD protein (e.g., A2M) (2) can be modified to be activated by one or more designated proteases (4), this technology allows the targeting of drugs to tissues expressing disease-associated proteases, e.g., diseased tissue, thereby potentially improving the efficacy of a drug while minimizing side effects arising from target binding in healthy tissues.

### Example 2—Production of Proteinaceous Fusion Constructs of A2M and Antibodies

### Aim

This data shows the expression and purification of A2M-antibody constructs as correctly folded tetrameric proteins, where A2M assumes a functional native conformation with a thiol ester.

### Materials and Methods

### Expression and Purification of A2M-Antibody Fusion Constructs

The nucleotide sequences encoding A2M-antibody fusion constructs and the corresponding amino acid sequences are given (SEQ ID NO: 5-22).

Proteinaceous fusion constructs were expressed in HEK293 FreeStyle cells using a standard transient transfection protocol. Briefly, 25 kDa linear polyethyleneimine (Polysciences) and plasmid DNA were incubated for 10 min in antibiotic-free FreeStyle medium (Thermo Fisher Scientific) at a 4:1 w/w PEI: DNA ratio, then slowly dripped into a culture of cells at a density of 1 million cells per mL, to a final DNA concentration of 1 μg per mL culture. After 4 days, the supernatant was harvested by spinning down the cells at 1500×g and adding pH 7.4 HEPES to a final concentration of 50 mM.

Purification of the constructs was performed using an established protocol for purifying A2M. Supernatants were first run through a Zn²⁺-loaded Chelating HiTrap column (GE Healthcare) and eluted with 50 mM EDTA, 150 mM NaCl, 100 mM sodium acetate, pH 7.4. The EDTA eluate was dialyzed against 20 mM HEPES at pH 7.4, then loaded onto a HiTrap Q column (GE Healthcare) and eluted by a gradient of 0-400 mM NaCl (with a constant 20 mM HEPES at pH 7.4). Fractions containing A2M were pooled, concentrated by ultrafiltration, and purified by size exclusion chromatography on a Sephacryl S-300 HR (GE Healthcare), using a 20 mM HEPES, 150 mM NaCl, pH 7.4 running buffer (HEPES-buffered saline, HBS).

### SDS-PAGE and Pore Limited Native PAGE

Native pore limited PAGE was performed as previously described (36), using homemade gels in TBE buffer (89 mM Tris, 89 mM boric acid, 2 mM EDTA) with an acrylamide gradient of 5-10% for A2M analysis and 10-15% for C3 analysis. Pore limited electrophoresis gels were run overnight at 100 V in TBE buffer.

Denaturing SDS-PAGE was performed using the discontinuous 2-amino-2-methyl-1,3-propanediol and glycine buffer system on homemade 5-15% acrylamide gradient gels. Samples were reduced with 25 mM DTT at 95° C. for 5 minutes.

Reaction of A2M with Methylamine and Proteases

To aminolyze A2M's thiol ester, methylamine (pH 8) was added to 250 mM and incubated for at 16 hours at 37° C. To assess the cleavage of A2M by thermolysin, thermolysin was added to a 2.2:1 mol/mol ratio of protease:A2M and incubated for five minutes at 37° C. The digestion was then inhibited using EDTA (10 mM, 15 minutes, room temperature).

### Results

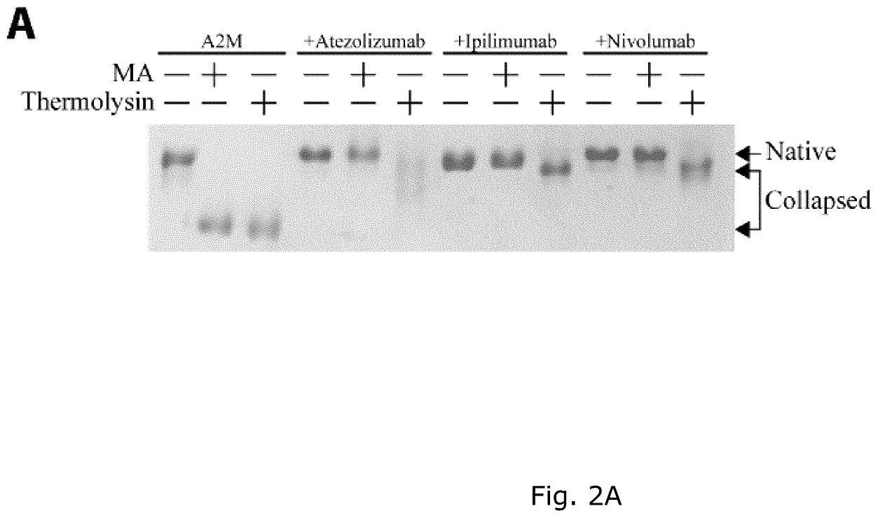

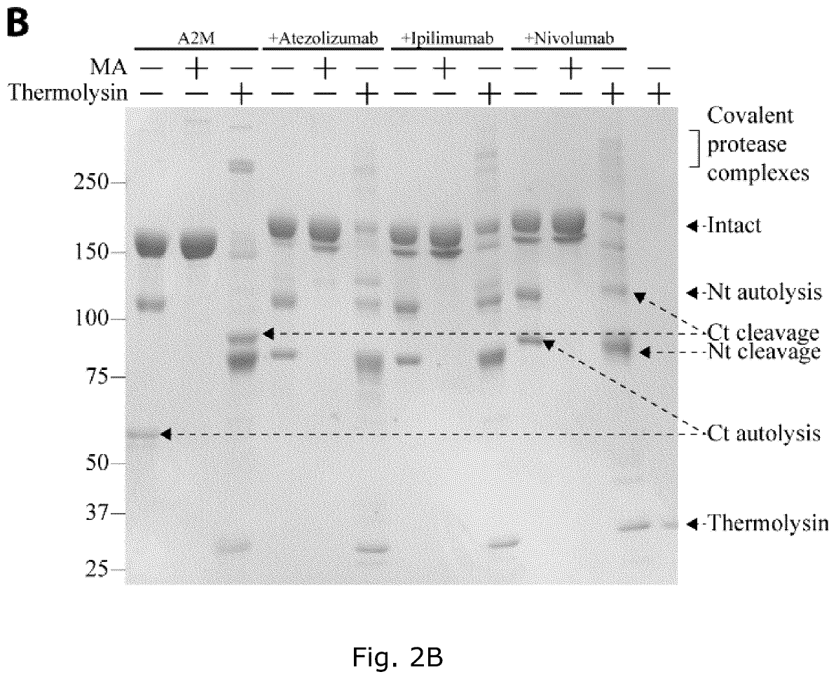

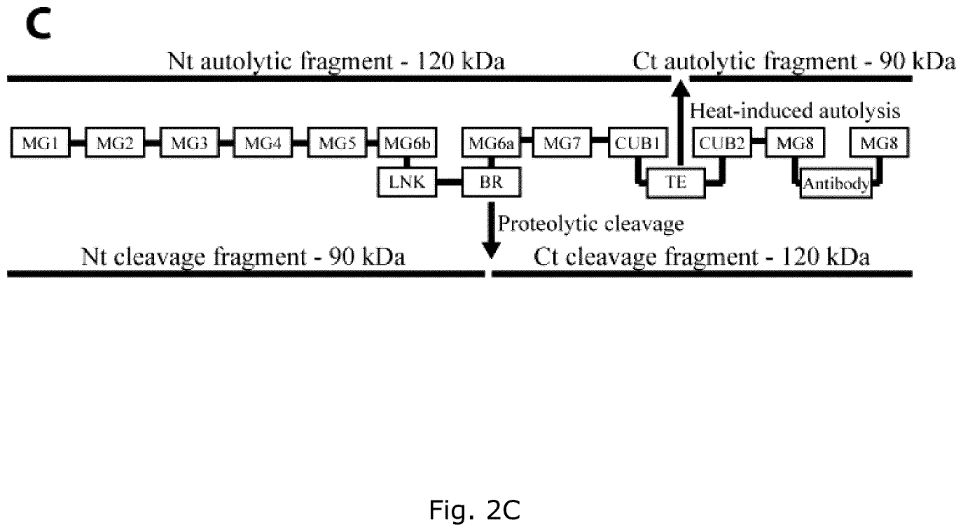

Proteinaceous fusion constructs were produced with yields of several mg/L in transient HEK293F transfections. After purification, the constructs migrated as native homotetramers in native PAGE (FIG. 2A), and as ≈190 kDa monomeric subunits in reducing, denaturing SDS-PAGE (FIG. 2B). The presence of a thiol ester in the constructs was verified by the presence of the characteristic heat-induced fragmentation at the site of the thiol ester, generating the Nt and Ct autolytic fragments visible in SDS-PAGE (FIG. 2B-C). These Nt and Ct autolytic fragments disappeared if the thiol ester was aminolyzed with methylamine prior to SDS-PAGE analysis. Proteolytic processing of the bait region was assessed by treatment with thermolysin. The bait regions of the constructs were preferentially cleaved by thermolysin, resulting in the formation of the Nt and Ct cleavage fragments (FIG. 2B-C). Bait region cleavage induced a conformational change in the constructs that increased their migration in native PAGE, although not to the same extent as in wildtype A2M (FIG. 2A); this is because the exposed antibody fragments increase the electrophoretic resistance experienced by activated A2M-antibody constructs.

### Conclusion

Proteinaceous fusion constructs of A2M and antibody scFvs are produced as homotetrameric proteins. In these proteins, the A2M component is functionally normal, as it assumes a native conformation, forms a thiol ester, and is preferentially cleaved in its bait region by proteases.

### Example 3—Conformational Dependence of Binding in Biolayer Interferometry

This example shows how the conformational change of the proteinaceous fusion construct is able to control the activity of the drug. In the native state, the drugs are not exposed and thus, inactive. In the active state, the drug is exposed and able to interact with its target.

### Aim

To determine the antigen-binding capacity of fusion constructs of A2M and antibodies, in binding experiments using purified antigens that are immobilized to biosensors, and to determine the extent to which this antigen-binding capacity is affected by the conformation of A2M.

### Materials and Methods

### Proteins Used for Binding Studies

Antigens to the antibodies under investigation were recombinantly expressed in HEK293F cells using a standard transient transfection protocol (see Example 1). The antigens were expressed with the leader peptide of A2M, N-terminal StrepII tags, and a C-terminal Fc region from human IgG1 (uniprot ID P01857, residues 100-330, SEQ ID NO: 40). The residues included for each antigen were as follows, using numbering before removal of the signal peptide:
EGFR (uniprot ID P00533, residues 25-645, SEQ ID NO: 37)
PD-L1 (uniprot ID Q9NZQ7, residues 19-239, SEQ ID NO: 38)
PD-1 (uniprot ID Q15116, residues 26-150 with a Cys93Ser mutation, SEQ ID NO: 39)
CTLA-4 (uniprot ID P16410, residues 36-161, SEQ ID NO: 40).
CD3γε (uniprot ID P09693, residues 23-103 of the γ chain followed by a 26-residue glycine-serine linker and uniprot ID P07766, residues 23-118 of the ε chain, SEQ ID NO: 41)
4-1BB (uniprot ID Q07011, residues 24-186, SEQ ID NO: 42)

The final sequences of these antigens in fusion as expressed are given both as amino acid and nucleotide sequences (SEQ ID NO: 45-58).

An additional antigen, TNFα (uniprot P01375, residues 77-233) was expressed as a StrepII-tagged protein but without a C-terminal Fc region (SEQ ID NO: 59, 60). It was also purified by StrepTactin affinity chromatography and size exclusion chromatography on a Superdex 200 Increase to isolate TNFα trimers.

Supernatants containing the expressed antigens were purified using StrepTactin affinity chromatography (Iba Life Sciences), followed by size exclusion chromatography on a Superdex 200 Increase (GE Healthcare).

The A2M-antibody fusion constructs were produced as stated in Example 1. Where stated, native A2M-antibodies were purified by affinity depletion of pre-activated A2M; see Example 5 for further details. The amino acid and nucleotide sequences of the A2M-antibodies are given (SEQ ID NO: 5-22).

Reaction of A2M-Antibodies with Methylamine and Proteases

When A2M-antibodies were treated with methylamine, 200 mM of methylamine (pH 8) was added to the A2M-antibody and it was incubated for 16 hours at 37° C. When A2M-antibodies were treated with thermolysin, thermolysin from Geobacillus stearothermophilus (Sigma-Aldrich) was added to the A2M-antibody at a 2.2:1 molar ratio of protease:A2M and incubated for 5 minutes at 37° C., after which point thermolysin was inhibited by the addition of 25 mM EDTA.

### Biolayer Interferometry

HEPES-buffered saline (HBS; 20 mM HEPES, 150 mM NaCl, pH 7.4) was used as the buffer in all biolayer interferometry experiments. Antigens were immobilized onto anti-human Fc capture biosensors (AHC biosensors; Fortebio) at 30 nM in HBS for 20 minutes. A2M-antibody fusion constructs were then incubated with the antigen-coated biosensors at various concentrations to measure association, followed by measurement of dissociation in HBS. Where stated, A2M-antibody fusion constructs were activated by methylamine or protease treatment, using the same method as in Example 1.

### Results

The conformational dependence of antigen binding by eight different A2M-antibodies was assessed by biolayer interferometry. Antigens were expressed as fusion proteins with the human IgG1 Fc region, allowing antigens to be immobilized onto the biosensor surface using anti-human Fc capture biosensors in a standardized manner. A2M-antibodies were then allowed to associate with their immobilized antigens, either without any treatment of the A2M-antibody or with the induction of A2M's conformational change using methylamine aminolysis and/or proteolysis by thermolysin. In some cases, the A2M-antibodies were enriched for the native conformation of A2M by affinity depletion using an antigen (PD-L1) or LRP1 resin, as noted in the figure legend and elaborated in Example 4.

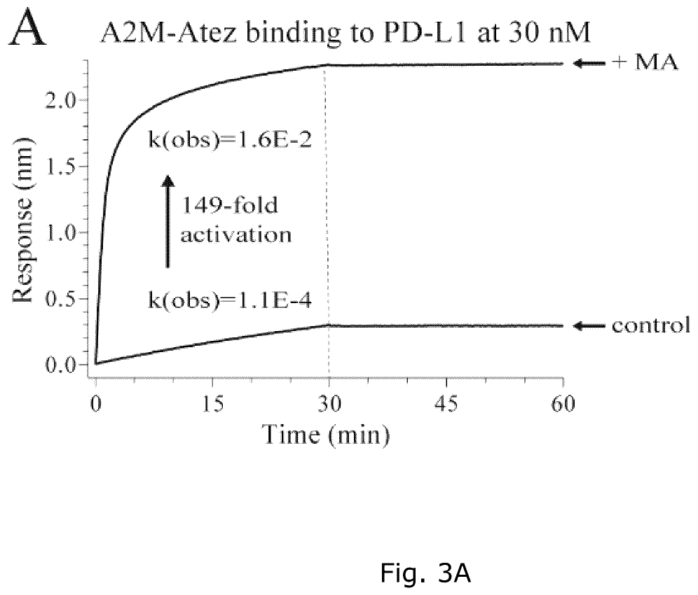

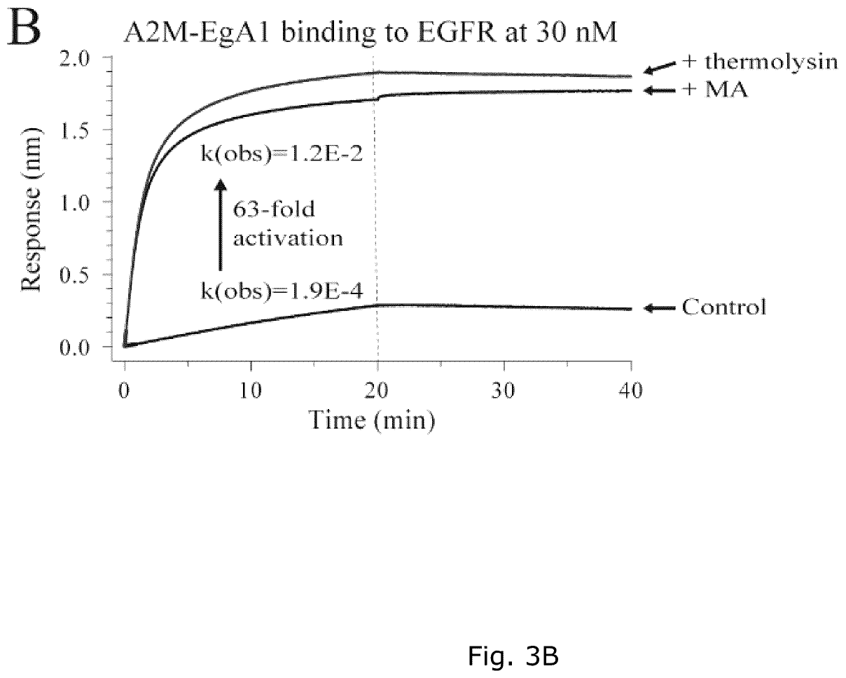

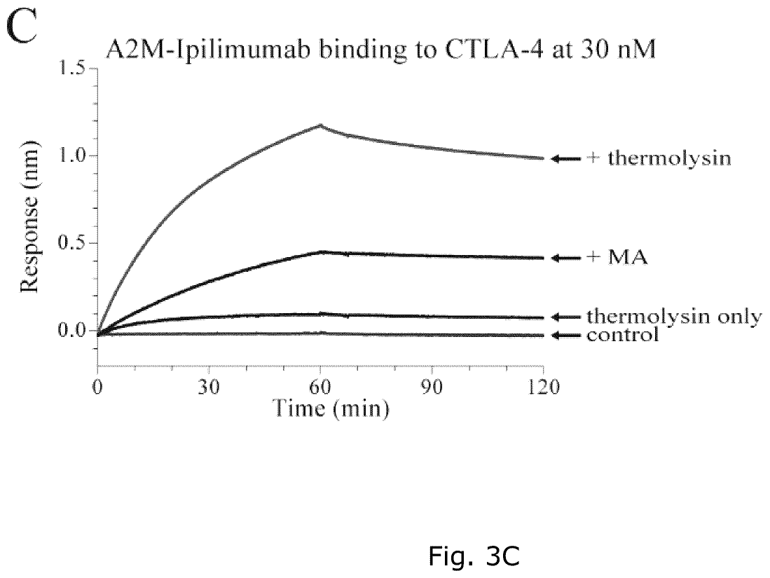

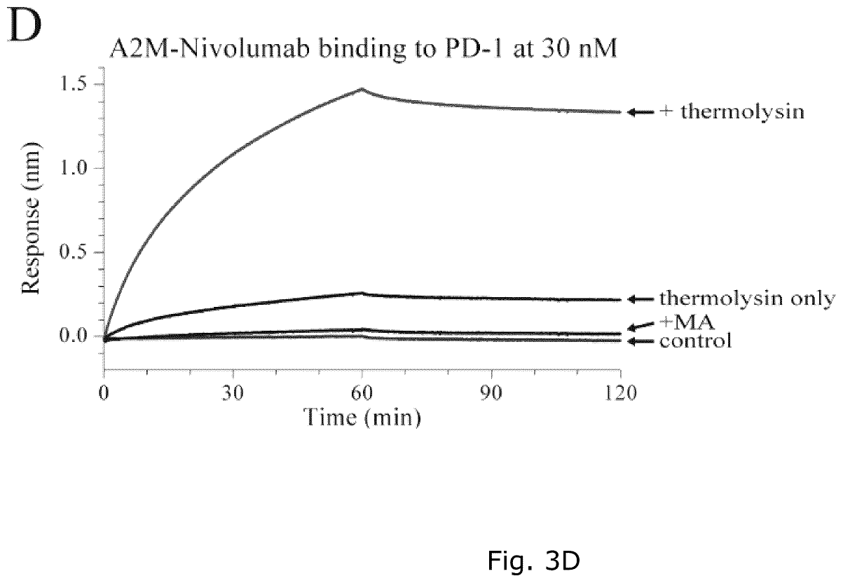

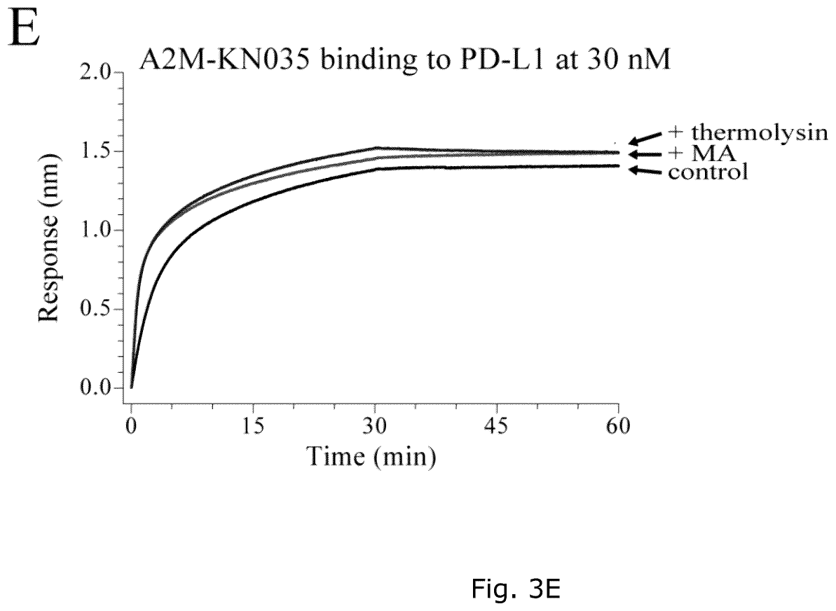

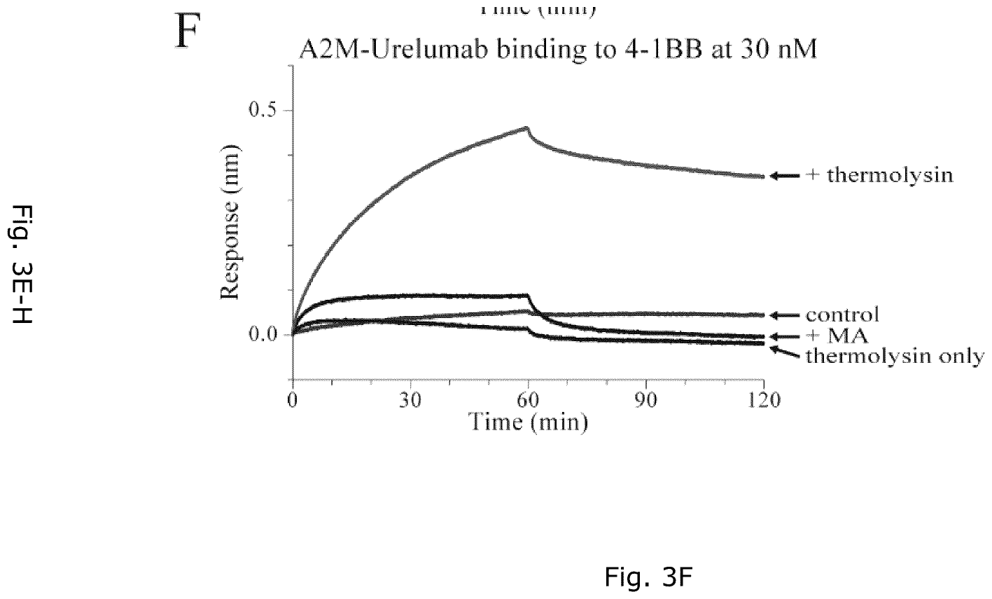

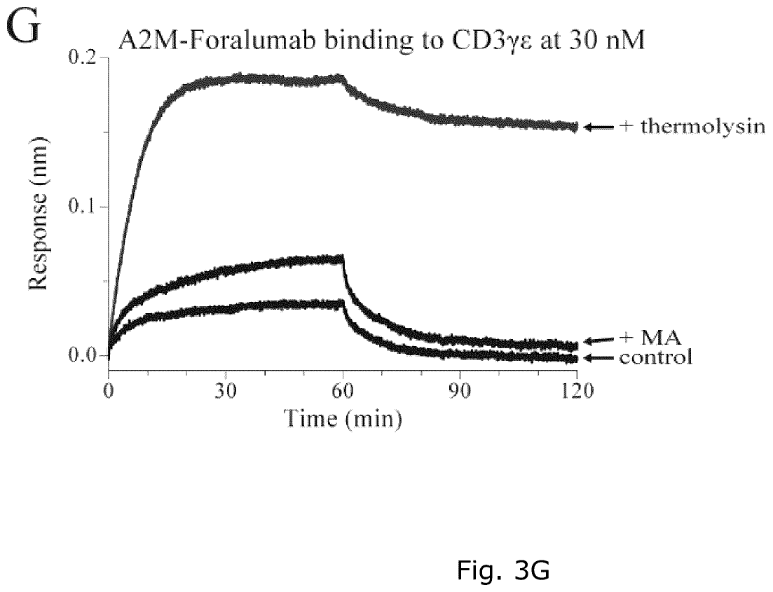

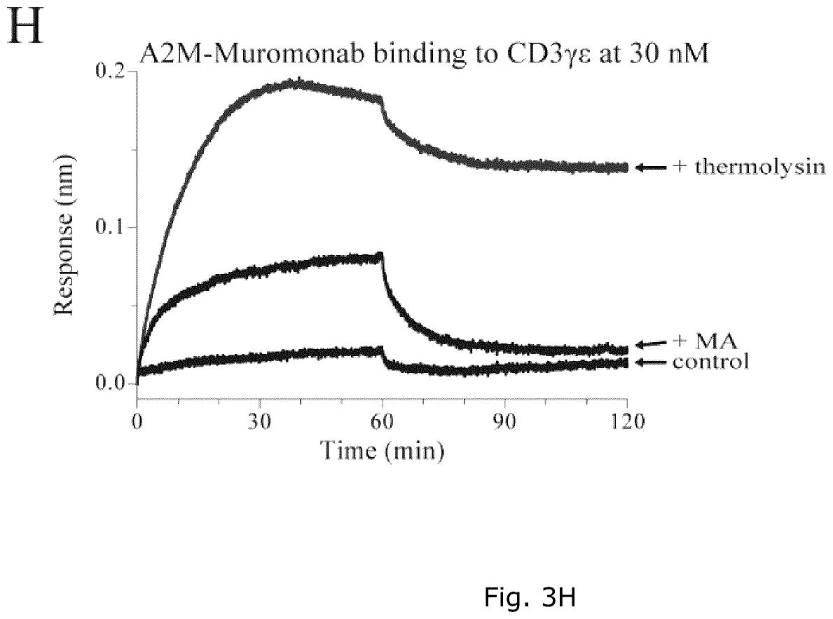

For all investigated antibodies, antigen binding was strongly dependent on the conformation of A2M (FIG. 3A-I), with thermolysin proteolysis of A2M consistently giving the highest binding response. Methylamine treatment produced variable binding responses; in some cases, methylamine induced a binding response similar to proteolysis (FIG. 3B, 3I), whereas in other cases it produced an intermediate response (FIG. 3C, 3F-H) or a neglible response (FIG. 3D). When the native conformation of an A2M-antibody had been enriched by affinity depletion, little to no antigen binding was detected in the native sample without methylamine/proteolysis (FIG. 3A-D, 3F).

### Conclusion

Antibodies incorporated into A2M fusion constructs retain the ability to bind their cognate antigen. This antigen binding is determined by the conformation of A2M, with little to no antigen binding in the native conformation of A2M. Activation of A2M by proteolytic cleavage greatly increases antigen binding, whereas activation by methylamine treatment varied depending on the A2M-antibody in question.

### Example 4—Enrichment of Native A2M-Antibody Constructs by Affinity Depletion

This example shows how modification of the proteinaceous fusion construct can be used to control where the drug becomes exposed. Depending on the cleavage site, the drug can only be exposed at the location where proteases recognizing that cleavage site are present. When a specific protease is present and cleaves the cleavage site, the conformation of the proteinaceous fusion construct is changed from “naïve” to “active”.

### Aim

Recombinantly expressed A2M-antibody fusion constructs are not exclusively produced with A2M in its native conformation; a minor component is produced in a pre-activated state. Here, it is investigated whether this pre-activated component can be removed by affinity depletion using the antibody's cognate antigen, the activated A2M receptor LRP1, or kappa light chain-binding Protein L.

### Materials and Methods

### Proteins Used

A2M-antibodies were produced as described in Example 1. Recombinant LRP1 (residues 20-974, SEQ ID NO: NO 63-64) was produced as a StrepII-tagged fusion protein with the human IgG1 Fc region, as described for the antigens in Example 2.

### Resin Preparation

A resin coated with LRP1 was prepared using amine reactive chemistry. A total of 200 mg of NHS-activated agarose (Pierce) and 600 μg of recombinant LRP1 in 0.15 M triethylammonium bicarbonate, 0.15 M HEPES, pH 8.3 were mixed on a rotator at room temperature for 2 hours.

Following incubation, the resin was washed twice in HBS and the reaction was quenched with 50 mM Tris-HCl, pH 8 for 20 minutes, followed by a final washing step with HBS.

A resin coated with PD-L1 was prepared as described for LRP1.

Protein L-coated agarose was purchased from Pierce (Thermo Scientific).

### Affinity Depletion

To deplete pre-activated A2M, A2M-antibody fusion constructs in HBS at up to 2 mg/mL were incubated with resin at room temperature overnight while shaken using a helicopter rotor. For LRP1-based depletion, 10 mM of CaCl₂) were added to the HBS. After overnight incubation, the supernatant was recovered and the resin was regenerated using HBS with 25 mM of EDTA in the case of LRP1, or using acidic elution with a pH 2.7, 10 mM KH₂PO₄ buffer for PD-L1 and Protein L.

The recovered supernatant was tested using biolayer interferometry, as described in Example 2.

### Results

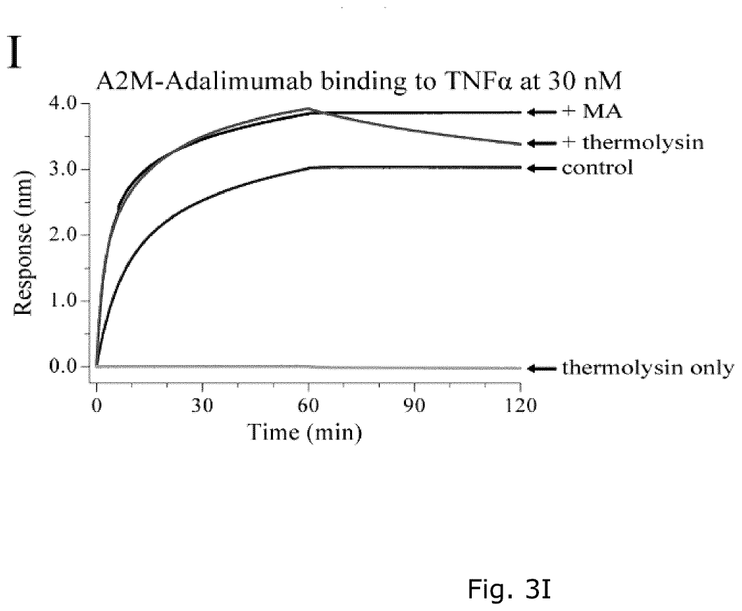

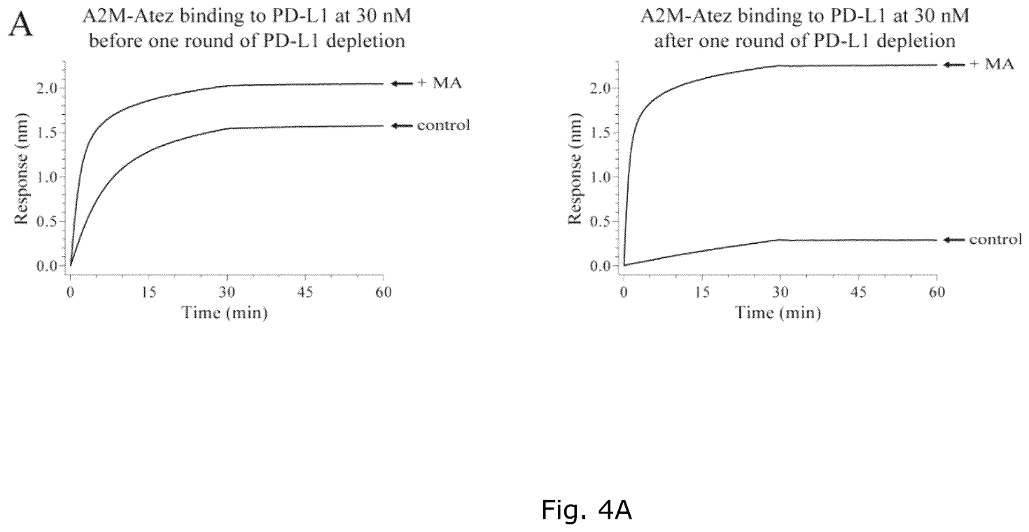

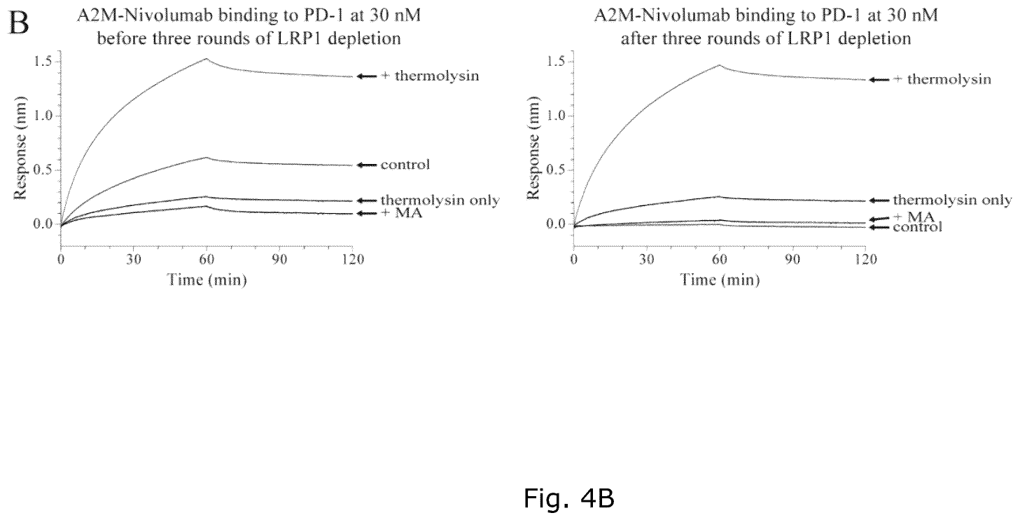

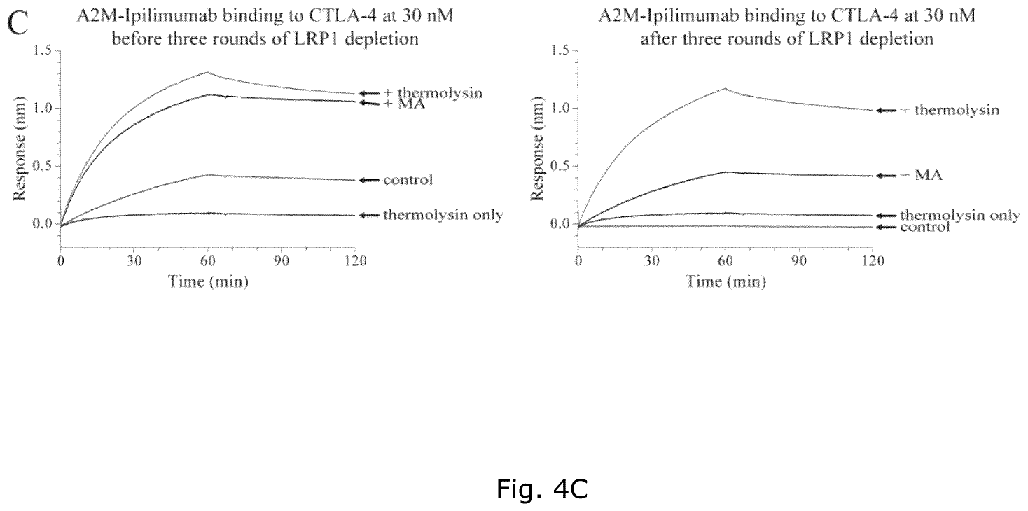

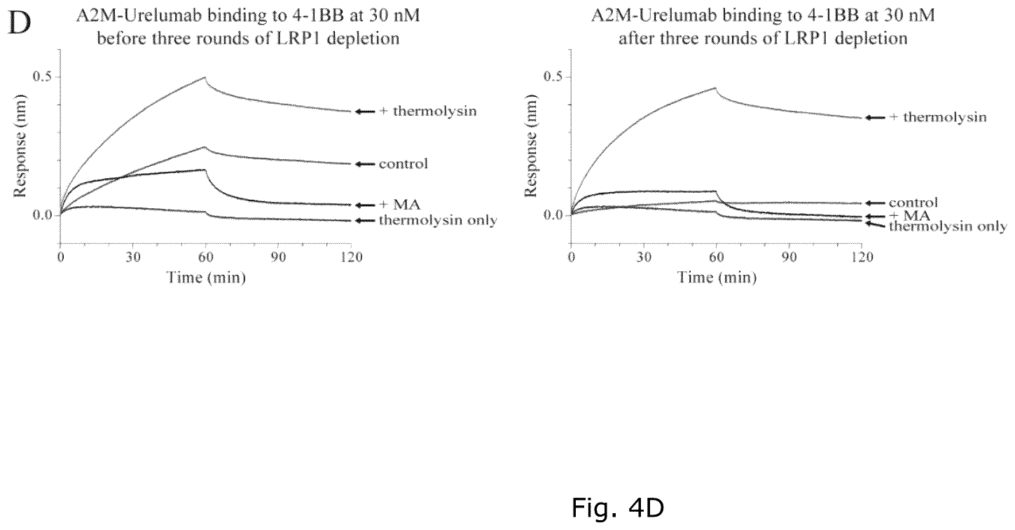

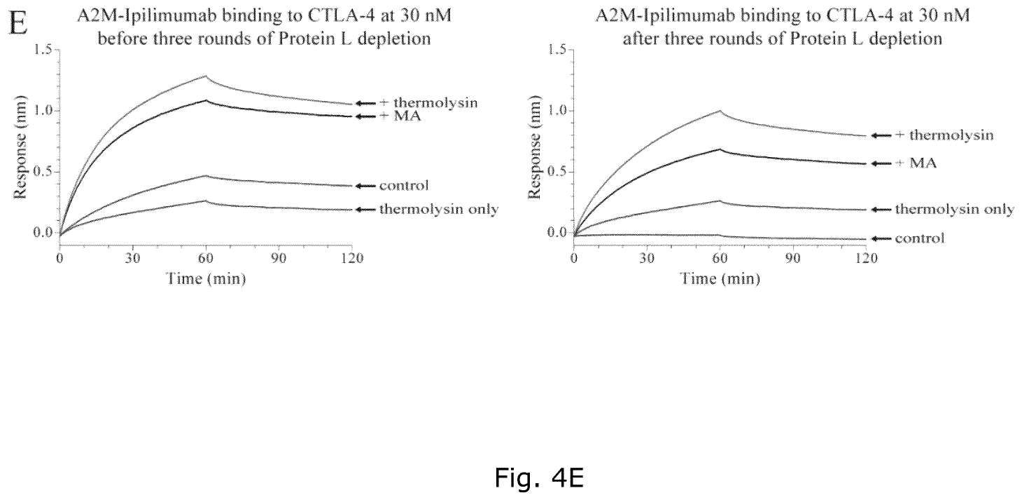

A2M-Atezolizumab was incubated with a resin coated with its cognate antigen, PD-L1. A single round of depletion was performed. The binding of A2M-Atezolizumab to PD-L1 before and after this depletion was then assessed using biolayer interferometry. Whereas A2M-Atezolizumab from before and after depletion bound similarly to PD-L1 upon methylamine treatment, antigen binding by the untreated sample after depletion was greatly decreased compared to the untreated sample before depletion, indicating that PD-L1 depletion had enriched the content of A2M-antibodies with inaccessible antibodies (FIG. 4A).

A2M-Ipilimumab, A2M-Nivolumab, and A2M-Urelumab were incubated with a resin coated with LRP1, a receptor that specifically binds to activated A2M but not to native A2M. Three rounds of depletion were performed for each A2M-antibody, after which biolayer interferometry was used to assess their binding to CTLA-4, PD-1, or 4-1BB, respectively (FIG. 4B-D). Antigen binding by the untreated A2M-antibodies before LRP1 depletion was approximately 25% of the maximum binding defined by thermolysin activation for all three antibodies. After LRP1 depletion, no antigen binding was detectable in the untreated A2M-Ipilimumab and A2M-Nivolumab samples and very little antigen binding was detectable in the untreated A2M-Urelumab sample, whereas equivalent binding was observed for the thermolysin-activated A2M-antibodies before and after LRP1 depletion. These data show that LRP1 depletion was able to deplete A2M-antibodies in which the antibodies were prematurely capable of antigen binding, further demonstrating that the conformation of A2M and the antibody accessibility are correlated.

A2M-Ipilimumab was also depleted using a Protein L-coated resin, which specifically binds to the κ light chain of human antibodies. Three rounds of depletion were performed. Biolayer interferometry showed that Protein L-based depletion was able to remove antigen binding in the untreated A2M-Ipilimumab sample (FIG. 4E).

### Conclusion

A2M-antibodies in their native and activated conformations can be distinguished by affinity depletion based on their binding to their antigen, to LRP1, or to Protein L. This binding can be used to remove activated A2M-antibodies and prepare native A2M-antibodies to a higher purity. Binding experiments comparing antigen binding before and after depletion show that the enrichment of native A2M-antibodies leads to minimal or no detectable antigen binding by the native protein, demonstrating that antigen binding by untreated A2M-antibodies is caused by contamination by non-native A2M-antibodies.

### Example 5—Investigating Immune Checkpoint Blockade in a Cell Assay

### Aim

In order to investigate whether A2M-antibodies demonstrate conformation-dependent target binding in a cellular context and retain the biological activity of their parent antibodies, A2M-Atezolizumab was investigated in a PD-1/PD-L1 blockade bioassay.

### Materials and Methods

### Proteins Used

A2M-Atezolizumab was expressed and purified as described for A2M-antibody fusion constructs in Example 2. Native A2M-Atezolizumab was enriched using PD-L1-based affinity depletion, as described in Example 4. Methylamine-treated A2M-Atezolizumab was prepared by 16 hours of incubation with 200 mM of methylamine at 37° C., followed by desalting back into HBS on a PD-10 column. The Atezolizumab scFv was also expressed in fusion with a human IgG1 Fc region, with N-terminal StrepII tags, and this Atezolizumab-hFc was purified using the same protocol as for antigen-hFc fusion constructs described in Example 3, namely StrepTactin affinity chromatography followed by size exclusion chromatography.

### Cell-Based Assessment of Immune Checkpoint Blockade

The ability of A2M-Atezolizumab and Atezolizumab-hFc to block the PD-1/PD-L1 pathway on human T cells was tested using the PD-1/PD-L1 Blockade Bioassay developed by Promega. Jurkat cells were cultured in RPMI 1640 medium supplemented with penicillin/streptomycin and 10% fetal bovine serum, while CHO-K1 cells were cultured in DMEM medium supplemented with penicillin/streptomycin and 10% fetal bovine serum. The day before performing the assay, 40*10³ CHO-K1 cells per well were seeded onto a 96-well plate. On the day of the assay, medium was removed from the wells and replaced by 40 μL of antibody solution diluted in assay buffer (RPMI 1640 medium with 1% fetal bovine serum) and 40 μL with 50*10³ Jurkat cells in assay buffer. The plates were then incubated at 37° C. for 6 hours, after which point 80 μL of Bio-Glo Reagent (Promega) were added to each well and luminescence was measured in a plate reader. Each antibody concentration and controls were tested in triplicate wells. The luminescence signal is given as averaged normalized luminescence for the three wells, with the background (measured from wells which did not receive any antibody) subtracted and the response normalized to the highest measured luminescence from the assay (with background subtracted).

### Results

The PD-1/PD-L1 Blockade bioassay developed by Promega was used to investigate conformation-dependent PD-L1 blocking by A2M-Atezolizumab. This bioassay uses the human Jurkat T cell line expressing human PD-1, as well as a luciferase reporter gene driven by an NFAT response element, to represent human T cells. CHO-K1 cells expressing human PD-L1 and an engineered surface protein that activates cognate TCRs in an antigen-independent manner are used to represent PD-L1⁺ target cells. The TCR-activating CHO-K1s would activate the Jurkat cells and induce a NFAT-driven luciferase response, except that this response is inhibited by PD-1-mediated signaling due to the engagement of PD-1 on the Jurkat cells by PD-L1 on the CHO-K1 cells. If either PD-1 or PD-L1 is blocked by an antibody, the luciferase response is restored.

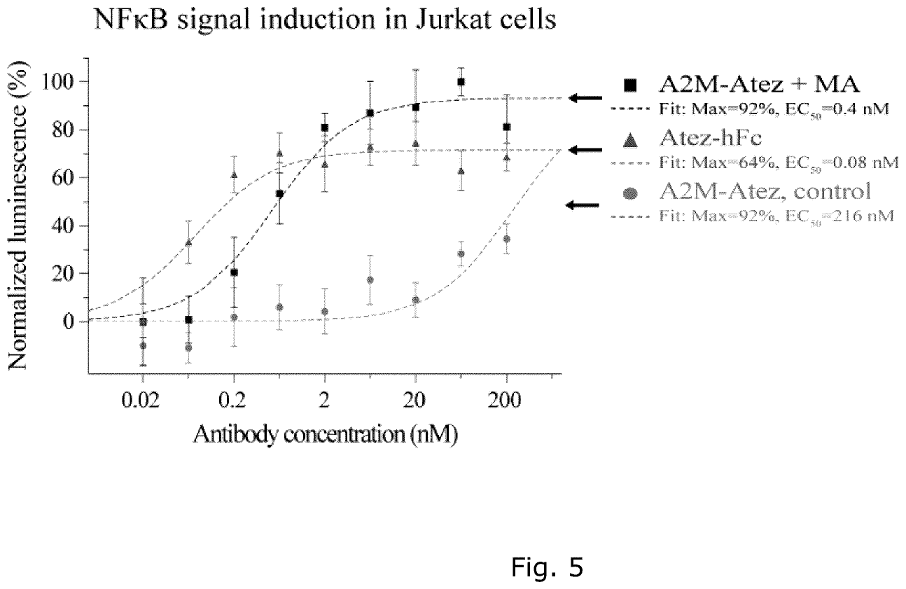

A titration series of A2M-Atezolizumab (from 20 pM to 200 nM) in its native conformation and methylamine-treated collapsed conformation was used to block PD-L1 on the surface of CHO-K1 cells. An IgG-resembling construct produced by fusing the Atezolizumab scFv to a human Fc region was included for comparison. A2M-Atezolizumab in both conformations and the Atezolizumab-hFc all produced a concentration-dependent luminescence response (FIG. 5). The maximum responses of methylamine-treated A2M-Atezolizumab and Atezolizumab-hFc were similar, whereas a saturated response for native A2M-Atezolizumab was not reached at the highest measured concentration of 200 nM (and its maximum response was therefore assumed to be the same as for methylamine-treated A2M-Atezolizumab). Both methylamine-treated A2M-Atezolizumab and Atezolizumab-hFc demonstrated sub-nanomolar EC₅₀ values of 400 and 80 pM, respectively, whereas native A2M-Atezolizumab had an EC50 value of 216 nM. There was therefore an approximately 500-fold difference in the activity of A2M-Atezolizumab in its native and activated conformations.

### Conclusion

A2M-Atezolizumab demonstrated a conformation-dependent ability to block PD-L1 and restore NFκB signaling in PD-1⁺ T cells in a cellular assay of immune checkpoint blockade. This demonstrates that A2M-Atezolizumab shows conformation-dependent binding to cell surface PD-L1 and that it retains the PD-L1-blocking functionality of the parent Atezolizumab antibody.

### Example 6—Modification of the A2M Bait Region to Target Specific Proteases

### Aim

The sequence of A2M's bait region determines whether it can be cleaved by a given protease, and thereby determines which proteases are able to activate A2M (and be trapped by A2M). The bait region of wildtype A2M can be cleaved by almost all human proteases and it would be advantageous to restrict the bait region's cleavage to designated proteases, to more specifically target diseased tissues. We first investigated whether the bait region can be replaced with a minimal sequence that does not contain cleavage sites for the majority of human proteases. We then investigated whether protease cleavage sites could be re-introduced into this minimal sequence, with the intent of producing bait region sequences with improved specificity for a single protease or family of proteases (in this example, matrix metalloproteases (MMPs)).

### Materials and Methods

### Proteins Used

A2M proteins with modified bait region sequences were expressed in HEK293F cells and purified as described for A2M-antibodies in Example 2. The amino acid sequences of these A2M proteins are given in SEQ ID NO: 65-73.

N-terminally StrepII-tagged proMMP2 (uniprot ID P08253, SEQ ID NO: 61-62) was expressed and purified using StrepTactin affinity chromatography and size exclusion chromatography, as described for StrepII-tagged hFc fusion proteins in Example 3.

ProMMP2 was activated using 1 mM APMA by incubating for 15 minutes at 37° C., followed by desalting into HBS with 10 mM CaCl2 using a PD-10 column (GE Healthcare).

### SDS-PAGE and Pore Limited Native Page

Native pore limited PAGE was performed as previously described (36), using homemade gels in TBE buffer (89 mM Tris, 89 mM boric acid, 2 mM EDTA) with an acrylamide gradient of 5-10% for A2M analysis and 10-15% for C3 analysis. Pore limited electrophoresis gels were run overnight at 100 V in TBE buffer.

Denaturing SDS-PAGE was performed using the discontinuous 2-amino-2-methyl-1,3-propanediol and glycine buffer system on homemade 5-15% acrylamide gradient gels (37). Samples were reduced with 25 mM DTT at 95° C. for 5 minutes.

Reaction of A2M with Methylamine and Proteases

To aminolyze A2M's thiol ester, methylamine (pH 8) was added to 250 mM and incubated for at least 45 minutes at 37° C. To assess the cleavage of A2M by trypsin and LysC, proteases were added to a 2.2:1 mol/mol ratio of protease:A2M and incubated for five minutes at 37° C. The digestion was then inhibited using the serine protease inhibitor PMSF (2 mM, 15 minutes, room temperature). To assess the cleavage of A2M by MMP2, MMP2 was added to A2M in HBS with 10 mM CaCl2 to a 6:1 mol/mol ratio of MMP2:A2M, incubated for 15 minutes at 37° C., and then inhibited using 20 mM EDTA. When cleaving A2M using other human proteases, incubation lasted one hour at 37° C. in HBS with 10 mM CaCl2, and PMSF or EDTA was used to inhibit serine proteases and metalloproteases, respectively.

### Determining A2M's Inhibition of Protein Substrate Cleavage by MMP2

The inhibition of MMP2 by A2M was investigated using a fluorescently labelled gelatin substrate. 1.4 pmol (7.5 nM) of MMP2 was reacted with 0-2.7 pmol (0-15 nM) of A2M in 50 mM HEPES, 100 mM NaCl, 5 mM CaCl₂) pH 8 for 15 min at 37° C. DQ Gelatin From Pig Skin (Invitrogen) was added to a final concentration of 0.1 mg/ml. The fluorescence (excitation at 485 nm and emission at 520 nm) of the unquenched digestion products of DQ gelatin after 10 min at 37° C. were measured in a FLUOstar Omega plate reader (BMG LABTECH). All reactions were performed in triplicates.

### Results

Bait Region Substitution with 13 Gly-Gly-Ser Triplets Produces A2M that is Tetrameric, Native, and Inducible.

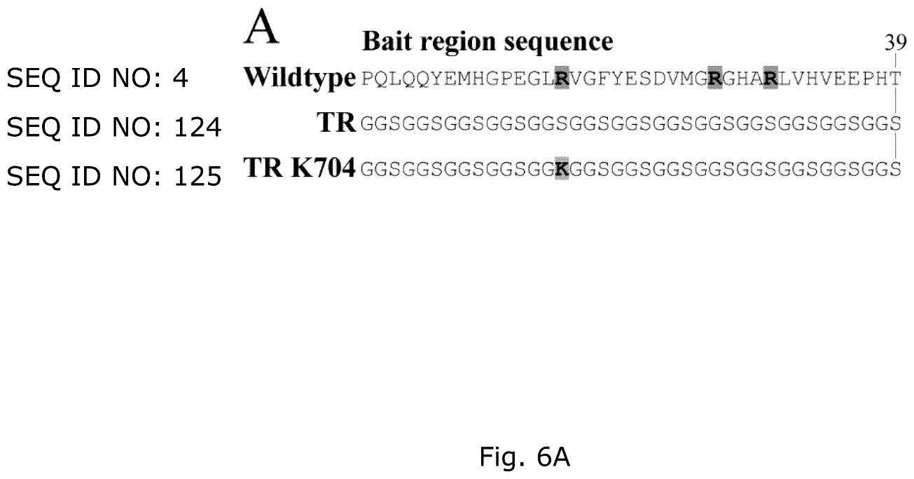

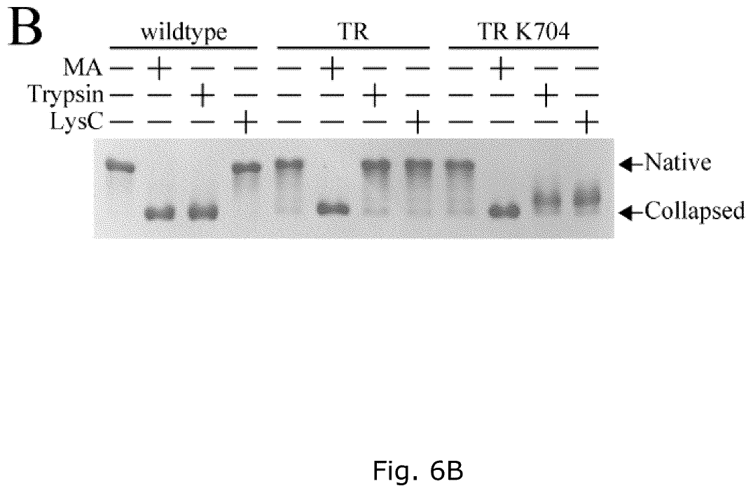

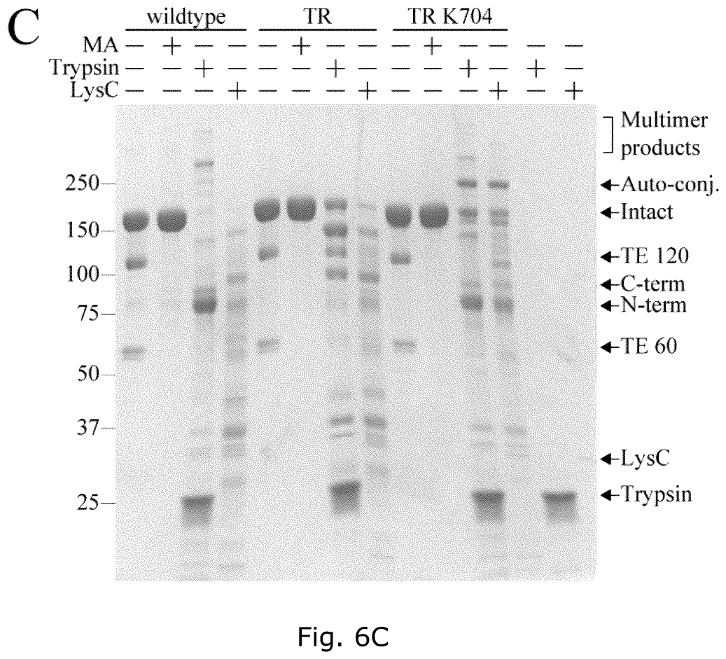

To remove essentially all protease cleavage sites from the bait region and determine the extent to which it tolerates modification, we replaced the 39-residue wildtype A2M bait region sequence with 13 Gly-Gly-Ser repeats, chosen for their solubility and low susceptibility to proteolysis (FIG. 6A). The resulting “tabula rasa” (TR) bait region was incorporated into recombinant A2M, resulting in A2M that was predominantly tetrameric and in its native conformation as assessed by native PAGE, with an intact thiol ester apparent from the formation of characteristic heat-induced autolysis products in SDS-PAGE (FIG. 6B-C). Upon aminolysis of its thiol ester with methylamine, TR A2M underwent a conformational collapse indistinguishable from that of wildtype A2M, as determined by pore-limited native PAGE; however, TR A2M was not cleaved in its bait region by either trypsin or LysC, and remained in its native conformation despite proteolysis by these proteases outside of its bait region (FIG. 6B-C). If a lysine residue was introduced into the TR bait region at position 704, the resulting bait region could be cleaved by both trypsin and LysC, resulting in protease conjugation and A2M's characteristic conformational change (FIG. 6A-C).

Identification of an MMP2-Cleavable Bait Region Sequence with Improved Selectivity.

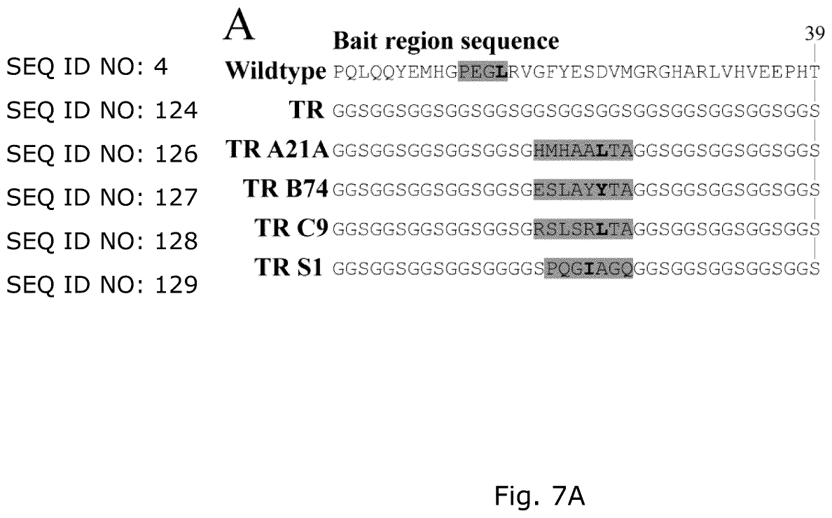

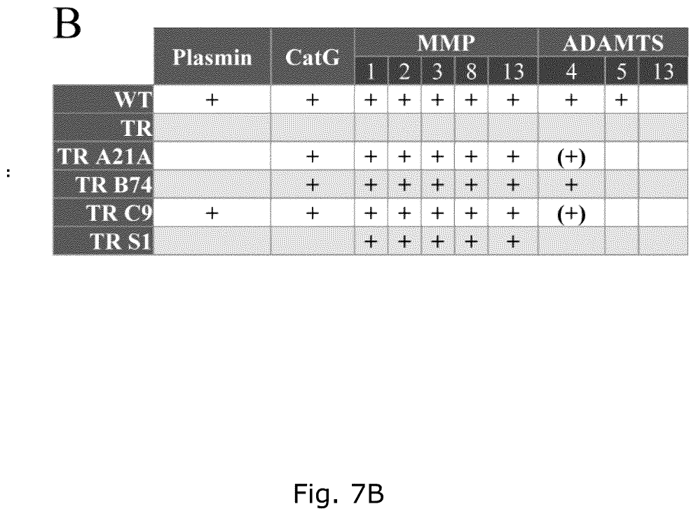

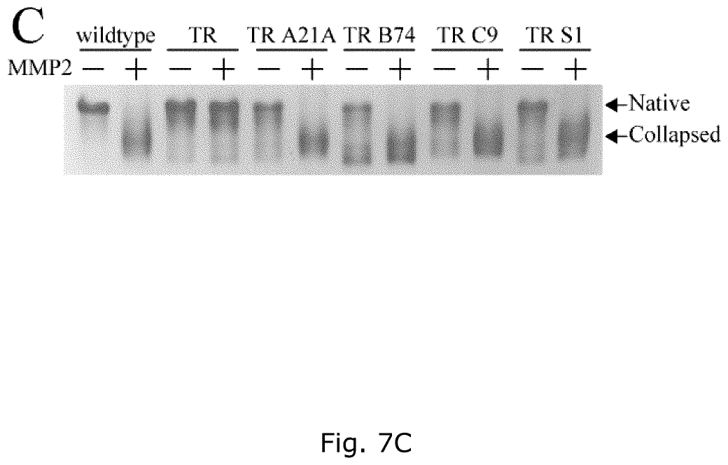

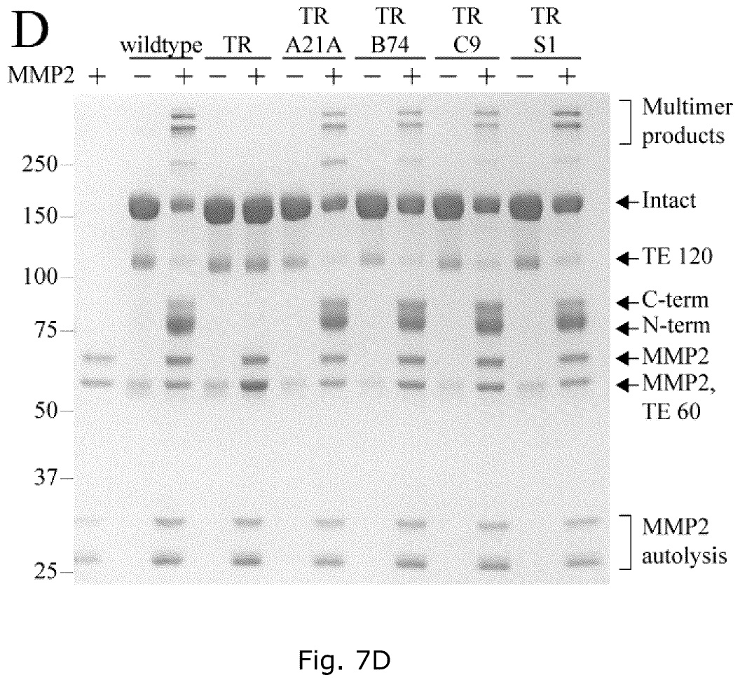

Four TR bait regions incorporating substrate sequences for human MMP2 were designed (FIG. 7A). All four TR-based MMP2 substrate bait regions and the wildtype bait region were cleaved by MMP2, but not the initial TR A2M (FIG. 7B-D). Incomplete bait region cleavage and intermediate electrophoretic mobility of A2M: MMP2 complexes was observed both for wildtype A2M and the four MMP2 substrate TR A2Ms.1

We then tested whether the four MMP2 substrate bait regions were cleaved by nine additional human proteases (plasmin, cathepsin G, MMP1, MMP3, MMP8, MMP13, ADAMTS4, ADAMTS5, and ADAMTS13) using reducing SDS-PAGE to assess bait region cleavage and the formation of high-MW conjugation products (FIG. 7B). All assessed proteases except ADAMTS13 (which is highly specific towards von Willebrand factor) were able to cleave wildtype A2M, while none were able to cleave TR A2M (FIG. 7B). The incorporation of any of the four MMP2 substrate sequences into the TR bait region conveyed cleavage by all tested MMPs, whereas they were differently cleaved by non-MMP proteases; for example, the C9 substrate was the only one containing an arginine residue and was the only A2M to be cleaved by plasmin (FIG. 7A-B). The S1 substrate was only cleaved by MMPs (FIGS. 7A-B), and the A2M TR S1 protein was therefore selected for further optimization as an A2M with an improved MMP specificity relative to wildtype A2M.

### The Native Content of Tabula Rasa-Based A2Ms is Improved by Shortening the Bait Region by Seven Residues or Restoring the 10 C-Terminal Wildtype Residues.

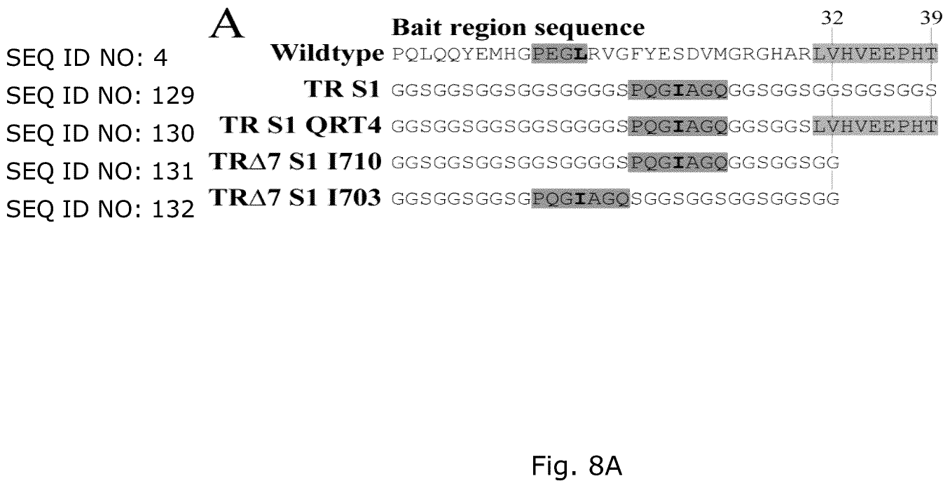

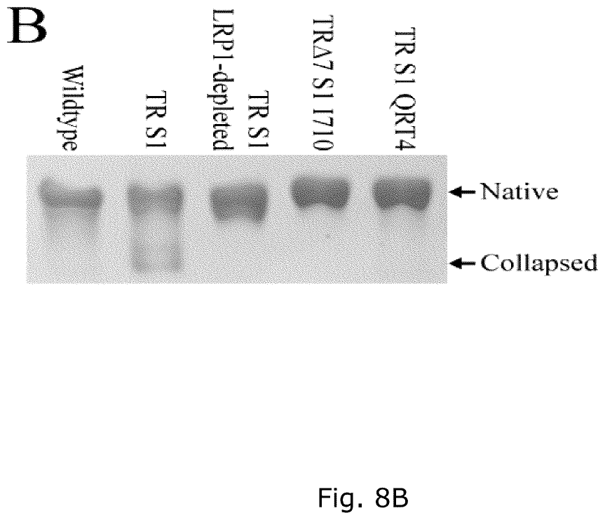

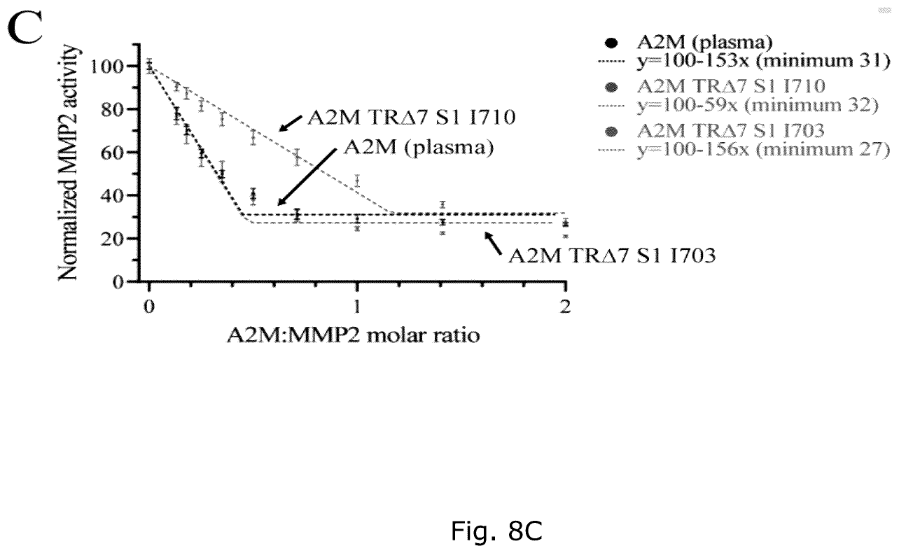

The initial TR A2M proteins were expressed with an increased amount of non-native A2M compared to wildtype A2M (FIG. 6A, 7C). To resolve this issue, we tested two altered tabula rasa bait regions where the first, TRΔ7, was shortened by 7 residues to a total length of 32 residues and the second, TR QRT4, re-introduced the C-terminal quarter of the wildtype bait region (FIG. 8A). Both TRΔ7 and TR QRT4 improved the native content of their resulting A2Ms to that of wildtype A2M (FIG. 8B). A position of the S1 substrate sequence in the TRΔ7 bait region was identified that conveyed an efficiency of MMP2 inhibition that was indistinguishable from that of wildtype A2M (FIG. 8C), showing that this shortened bait region can produce fully functionally A2M.

### Conclusion

The bait region of A2M could be completely replaced by glycine and serine residues without compromising the structure and function of A2M, although a glycine-serine bait region that was shortened to 32 residues was found to give an improved yield of native A2M. The glycine-serine bait region was not cleavable by 10 tested human proteases. Upon incorporation of the S1 substrate for MMP2 into the bait region, 5 human MMPs were able to cleave the bait region, while 5 non-MMPs remained unable to cleave. This demonstrates that the glycine-serine bait region can be used as the foundation for making bait regions with an improved specificity to a protease or family of proteases (such as MMPs).

### Example 7—Bait Region Modification of A2M-Antibodies

### Aim of Study

In Example 6, bait region sequences based on the “tabular rasa” (TR) bait region which replaces the wildtype bait region with glycine and serine residues were found to produce A2M proteins which were more specifically cleaved and activated by target proteases. Furthermore, a TR bait region that was shortened by 7 residues (TRΔ7) to a length of 32 residues was found to convey an increased yield of native A2M, and the placement of the S1 substrate for MMP2 into TRΔ7 at a specific position (TRΔ7 S1 I703) was found to convey an inhibition of MMP2 that was equivalent to that of the wildtype A2M bait region. Here, we investigated whether A2M-antibodies incorporating TR bait regions with MMP2 substrate sites could be activated by MMP2 in the same manner as A2M-antibodies with wildtype bait regions.

### Materials and Methods

### Proteins Used

A2M-Atezolizumab with the wildtype bait region (SEQ ID NO: 7-8), the TRΔ7 S1 I703 bait region (SEQ ID NO: 74-75), or the TRΔ7 S1 I703 P704 bait region (SEQ ID NO: 76-77) were expressed in HEK293F cells and purified as described for A2M-antibodies in Example 2. ProMMP2 was expressed, purified, and activated as described in Example 6.

### Cleavage of A2M by Proteases

To cleave A2M-antibodies with MMP2, MMP2 was added in HBS with 10 mM CaCl2 to a 4:1 mol/mol ratio of MMP2:A2M, incubated for 15 minutes at 37° C., and then inhibited using 20 mM EDTA. To cleave A2M-antibodies with thermolysin, thermolysin was added in HBS with 10 mM CaCl2 to a 2.2:1 mol/mol ratio of thermolysin:A2M, incubated for 2 minutes at 37° C., and then inhibited using 20 mM EDTA.

### Biolayer Interferometry

Biolayer interferometry was used to investigate the interaction between A2M-Atezolizumab with different bait regions using the method described in Example 3.

### SDS-PAGE and Pore Limited Native PAGE

Native pore limited PAGE was performed as previously described (36), using homemade gels in TBE buffer (89 mM Tris, 89 mM boric acid, 2 mM EDTA) with an acrylamide gradient of 5-10% for A2M analysis and 10-15% for C3 analysis. Pore limited electrophoresis gels were run overnight at 100 V in TBE buffer.

Denaturing SDS-PAGE was performed using the discontinuous 2-amino-2-methyl-1,3-propanediol and glycine buffer system on homemade 5-15% acrylamide gradient gels (37). Samples were reduced with 25 mM DTT at 95° C. for 5 minutes.

### Results

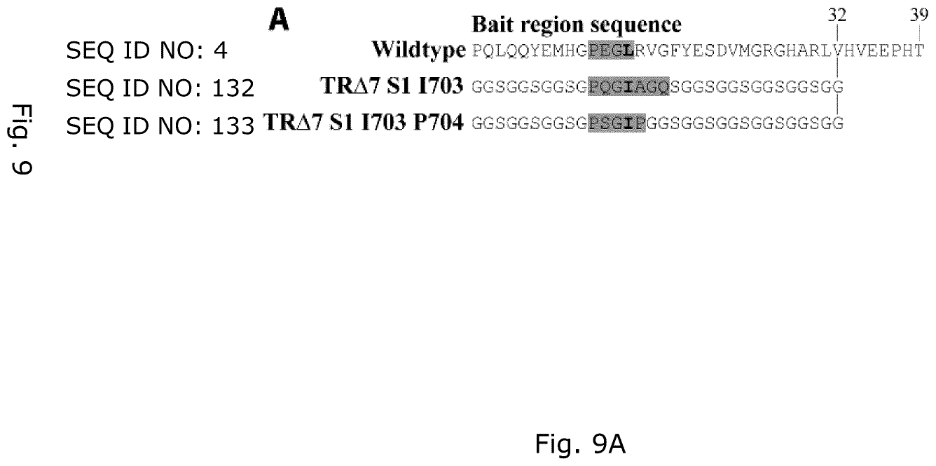

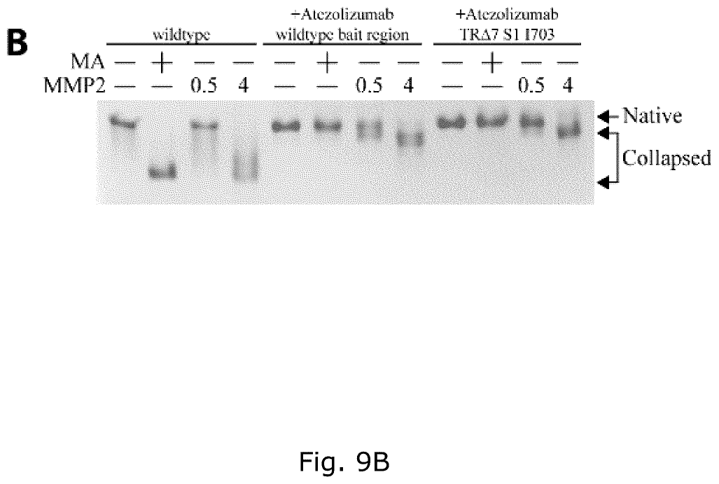

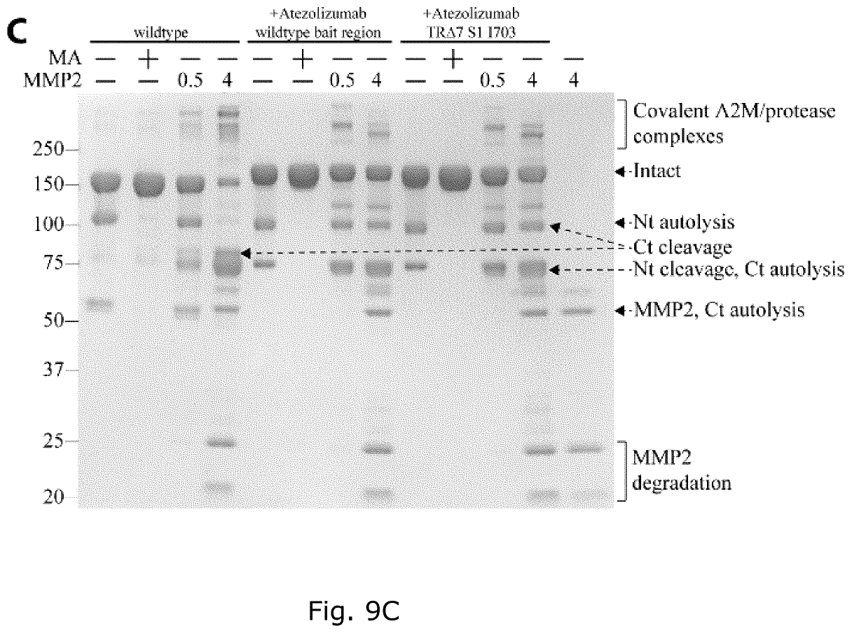

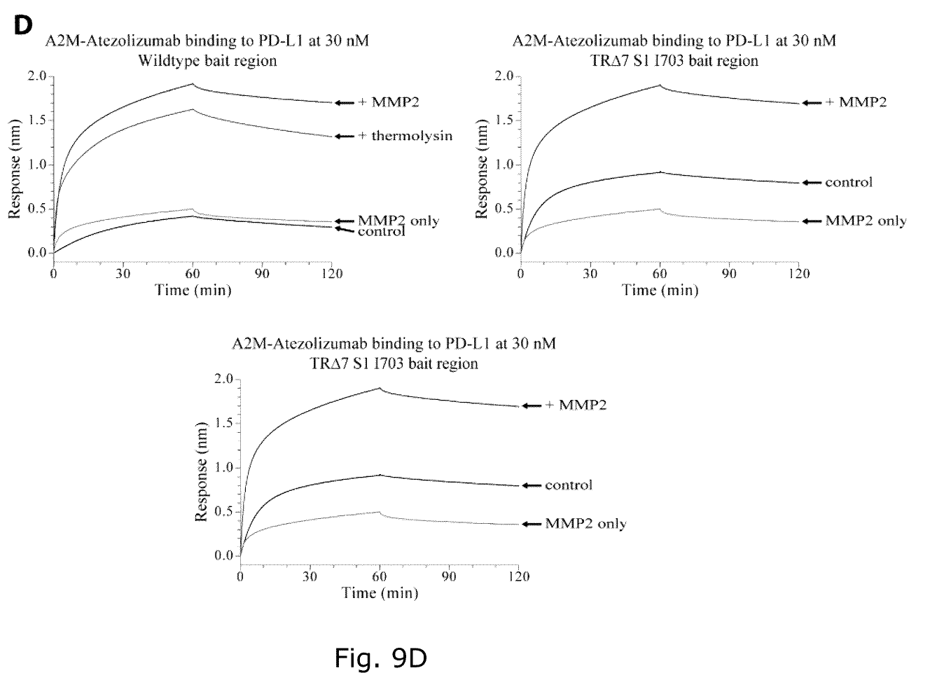

To assess the functionality of A2M-antibodies with engineered bait regions, A2M-Atezolizumab was expressed with either a wildtype bait region, the TRΔ7 S1 I703 bait region (an optimized MMP2-substrate bait region described in Example 6), or the TRΔ7 S1 I703 P704 bait region which minimizes the MMP2 cleavage site and prevents cleavage of residue I703 by serine proteases by adding a P′1-position proline residue (FIG. 9A). All three A2M-Atezolizumab proteins were bait region cleaved when treated with MMP2, as assessed by their conformational change in native PAGE (FIG. 9B) and cleavage of the A2M subunit in reducing SDS-PAGE (FIG. 9C). These results show that A2M-antibodies with engineered MMP2-substrate bait regions are cleaved in their bait regions by MMP2.

Biolayer interferometry was used to investigate the effect of MMP2 cleavage on the A2M-Atezolizumab proteins' binding to immobilized PD-L1. MMP2 cleavage was found to convey a similar antigen binding to that induced by thermolysin cleavage, for A2M-Atezolizumab with a wildtype bait region (FIG. 9D). Both A2M-Atezolizumab proteins with engineered bait regions showed similar antigen binding upon their cleavage with MMP2 (FIG. 9D). These results show that bait region cleavage by MMP2 induces antigen binding in A2M-antibodies, both with wildtype bait regions and with engineered MMP2-substrate bait regions.

### Conclusion

Engineered bait regions, as described in Example 5, can be incorporated into A2M-antibodies without disrupting their conformationally dependent antigen binding, and are still preferentially cleaved by target proteases such as MMP2. MMP2 cleavage is able to induce antigen binding in A2M-antibodies with wildtype bait regions or engineered bait regions.

### Example 8—Incorporation of the Extracellular Region of PD1 Receptor into A2M

### Aim of Study

Here, we investigate whether the extracellular region of the human PD1 receptor can be incorporated into A2M (in the same manner as antibodies, as shown in previous examples) and whether the resulting A2M-PD1 fusion protein binds to the PD1 receptor's ligand, PD-L1, in a manner that is dependent on the conformation of A2M.

### Material and Methods

### Proteins Used

The A2M-PD1 fusion construct was expressed and purified as described for A2M-antibodies in Example 1. The extracellular region of PD1 that was incorporated into A2M was the same sequence used for testing A2M-nivolumab in Example 2, i.e. uniprot ID Q15116, residues 26-150 with a Cys93Ser mutation. The amino acid and nucleotide sequence of A2M-PD1 is given in SEQ ID NO: 25-26. PD-L1 fused to a human Fc region was prepared as described in Example 2.

### SDS-PAGE and Pore Limited Native PAGE

A2M-PD1 was analyzed by reducing SDS-PAGE using the protocol described in Example 2.

Reaction of A2M-Antibodies with Methylamine and Proteases

A2M-PD1 was treated with methylamine or the metalloprotease thermolysin in order to change its conformation, as described in Example 3.

### Biolayer Interferometry

Biolayer interferometry was used to investigate the binding of A2M-PD1 in its untreated, methylamine-, or thermolysin-treated conformations to PD-L1 immobilized on the surface of biosensors, as described in Example 3.

### Results

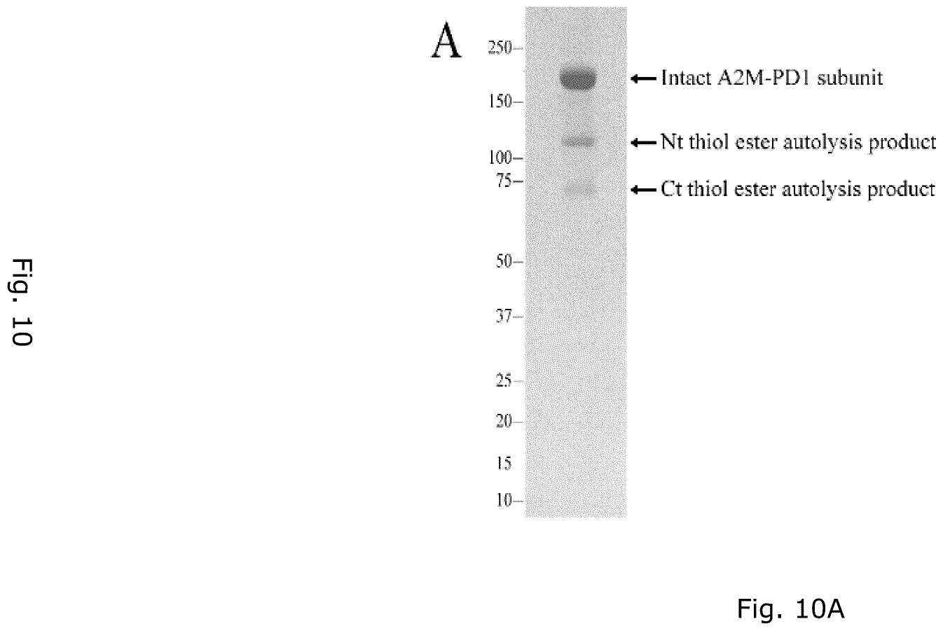

Using the same fusion strategy that was used to incorporate antibody scFvs and nanobodies into A2M, the extracellular region of human PD1 was incorporated into A2M and the resulting A2M-PD1 was expressed and purified using standard A2M protocols (FIG. 10A).

The conformational dependence of PD-L1 binding by A2M-PD1 was then assessed by biolayer interferometry. PD-L1 binding by A2M-PD1 was strongly dependent on the conformation of A2M (FIG. 10B). A control sample with untreated A2M-PD1 showed a minor degree of binding, as native A2M-PD1 was not purified from non-native A2M-PD1 prior to this experiment, e.g. using LRP1-based depletion as described in Example 3. In contrast, both methylamine- and thermolysin-treated A2M showed greatly enhanced binding responses, and these responses were very similar to each other. When A2M-PD1 in its native conformation was enriched by three rounds of LRP1-based depletion of non-native A2M-PD1, no binding by untreated A2M-PD1 was detectable.

### Conclusion

PD1 could be incorporated into A2M, resulting in functional A2M that was capable of undergoing its typical methylamine- and thermolysin-induced conformational changes and PD1 that was capable of binding to its ligand, PD-L1. Furthermore, the binding of PD1 to PD-L1 was dependent on the conformation of A2M, with no detectable binding of A2M-PD1 in its native conformation to PD-L1.

### Example 9—Incorporation of the IL2 Cytokine into A2M

### Aim

Here, we investigate whether the IL2 cytokine can be incorporated into A2M (in the same manner as antibodies, as shown in previous examples) and whether the resulting A2M-IL2 fusion protein binds to an IL 2 receptor, IL-2Rα, in a manner that is dependent on the conformation of A2M.

### Material and Methods

### Proteins Used

The A2M-IL2 fusion construct was expressed and purified as described for A2M-antibodies in Example 1. The IL2 cytokine that was incorporated into A2M used the wildtype human sequence (uniprot P60568, residues 21-153). The amino acid and nucleotide sequence of A2M-IL2 is given in SEQ ID NO: 23-24.

The extracellular region of the IL2 receptor IL-2Rα (uniprot P01589, residues 22-238, with a Cys213Ala mutation, SEQ ID NO: 43) was expressed as a Strep-Tagged human Fc fusion protein (SEQ ID NO: 57-58), as described for other antigens in Example 2, and purified in the same manner as well.

### SDS-PAGE and Pore Limited Native PAGE

A2M-IL2 was analyzed by reducing SDS-PAGE using the protocol described in Example 2.

Reaction of A2M-Antibodies with Methylamine and Proteases

A2M-IL2 was treated with methylamine or the metalloprotease thermolysin in order to change its conformation, as described in Example 3.

### Biolayer Interferometry

Biolayer interferometry was used to investigate the binding of A2M-IL2 in its untreated, methylamine-, or thermolysin-treated conformations to IL-2Rα immobilized on the surface of biosensors, as described in Example 3.

### Results

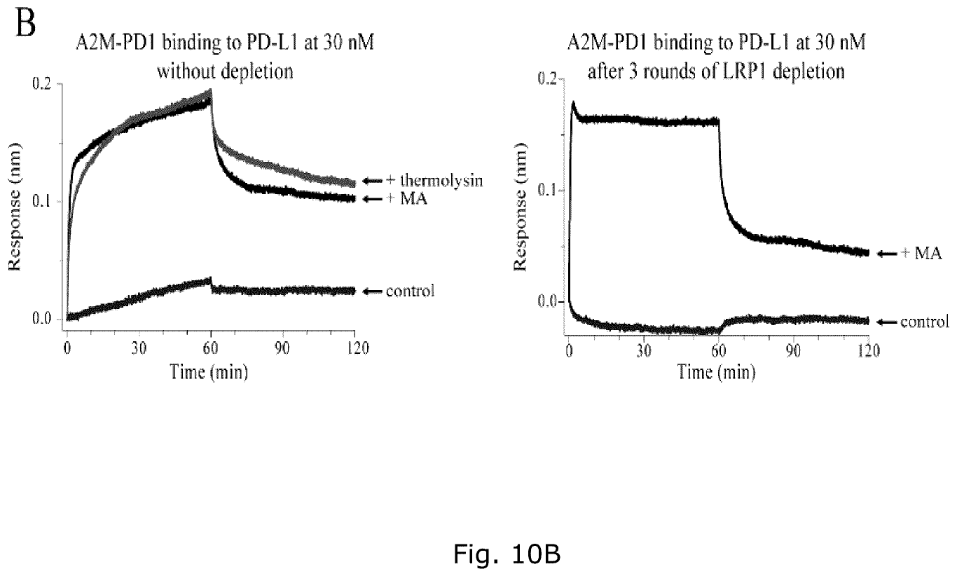

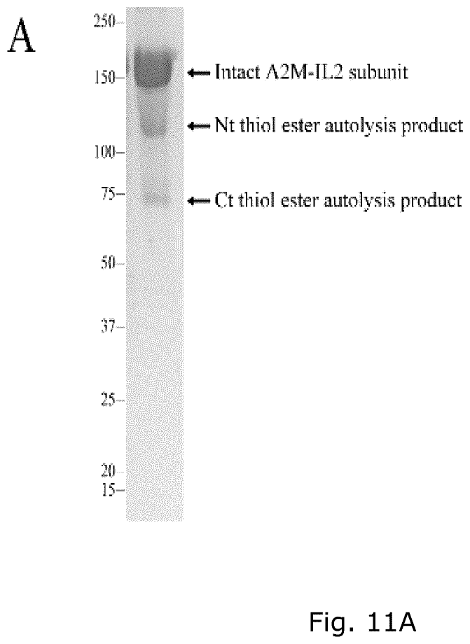

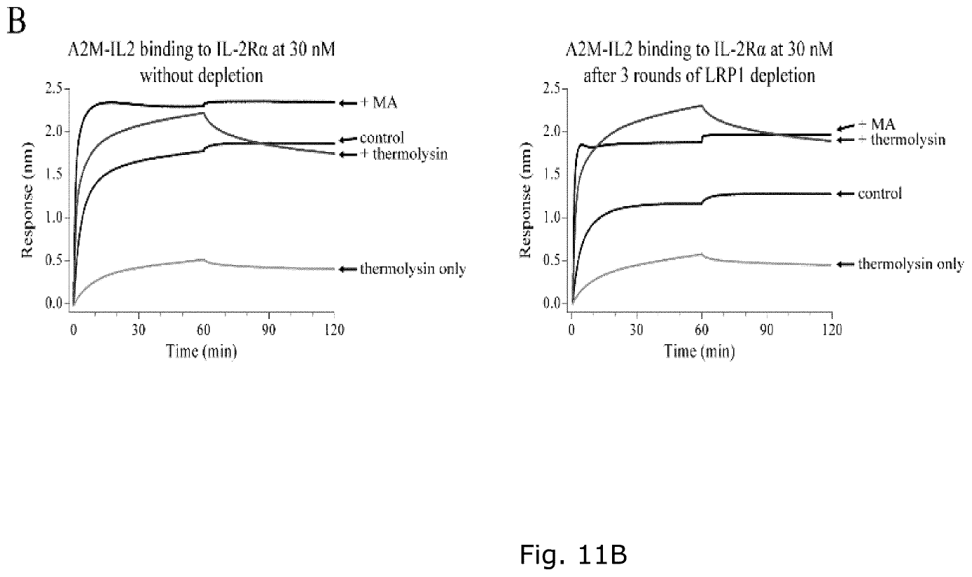

Using the same fusion strategy that was used to incorporate antibody scFvs and nanobodies into A2M, the human cytokine IL2 was incorporated into A2M and the resulting A2M-IL2 was expressed and purified using standard A2M protocols (FIG. 11A).

The conformational dependence of IL-2Rα binding by A2M-IL2 was then assessed by biolayer interferometry. IL-2Rα binding by A2M-IL2 was dependent on the conformation of A2M (FIG. 11B). A control sample with untreated A2M-IL2 showed a moderate degree of binding, even after using LRP1-based depletion of non-native A2M-IL2. Cleavage of the A2M bait region with thermolysin conferred an intermediately increased rate of association and saturation of binding, whereas aminolysis of the A2M thiol ester with methylamine conferred an even greater increase in both association rate and binding saturation. The degree of binding (as determined by k_{obs} values) is approximately 10-fold increased by methylamine treatment, compared to A2M-IL2 in its native conformation.

### Conclusion

IL2 could be incorporated into A2M, resulting in functional A2M-IL2 that underwent its typical methylamine- and thermolysin-induced conformational changes and IL2 that was capable of binding to the receptor IL-2Rα. Furthermore, this receptor binding by A2M-IL2 was dependent on the conformation of A2M, with increased receptor binding observed upon collapse of the A2M conformation by bait region cleavage or thiol ester aminolysis.

### Example 10—Investigating Other Fusion Strategies for the Incorporation of Biopharmaceutical Moieties into A2M

### Aim

The previous examples investigating proteinaceous fusion constructs of A2M and biopharmaceutical moieties use the ciRBD approach, where the biopharmaceutical moieties are inserted between A2M residues 1402 and 1403. Here, we investigate whether target binding that is dependent on the conformation of A2M can be achieved by four other approaches: fusion, iRBD, miRBD, and tRBD.

### Materials and Methods

### Proteins Used

All fusion constructs of A2M and biopharmaceutical moieties were expressed and purified as described for A2M-antibody fusion constructs in Example 2. No depletion of non-native A2M was performed. The amino acid and nucleotide sequences of A2M-fusion-EgA1 (SEQ ID NO: 94-95), A2M-iRBD-EgA1 (SEQ ID NO: 84-85), A2M-miRBD-EgA1 (SEQ ID NO: 86-87), A2M-miRBD-Atezolizumab (SEQ ID NO: 88-89), A2M-miRBD-KN035 (SEQ ID NO: 90-91), and A2M-tRBD-EgA1 (SEQ ID NO: 92-93) are given. EGFR and PD-L1 in fusion with a human FC region (SEQ ID NO: 45-48) were produced as described in Example 3.

Reaction of A2M-Antibodies with Methylamine and Proteases

A2M-antibodies were reacted with methylamine or proteases as described in Example 3.

### Biolayer Interferometry

Biolayer interferometry was performed as described in example 3.

### Results

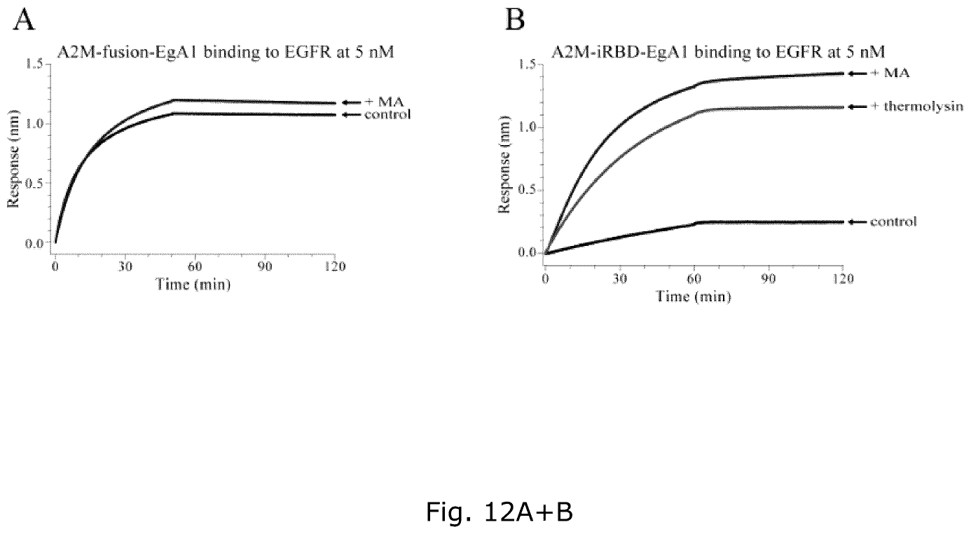

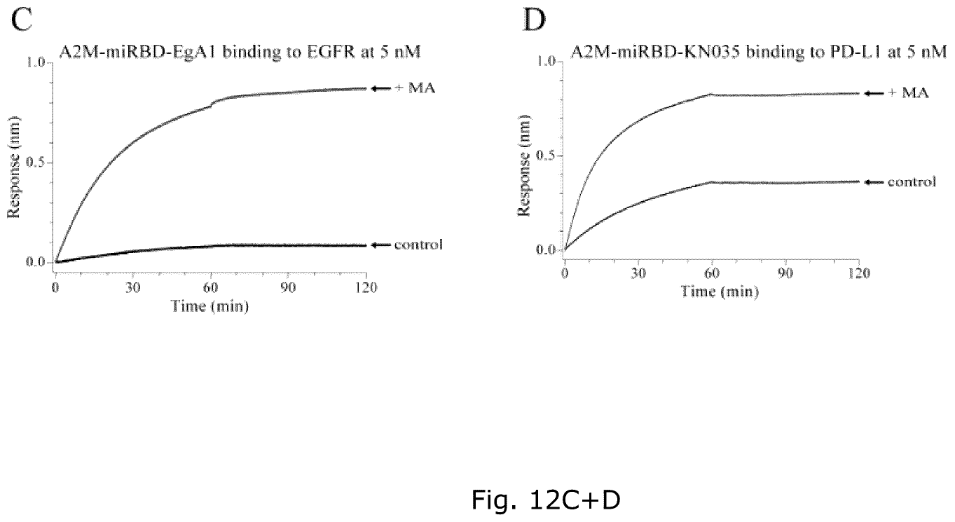

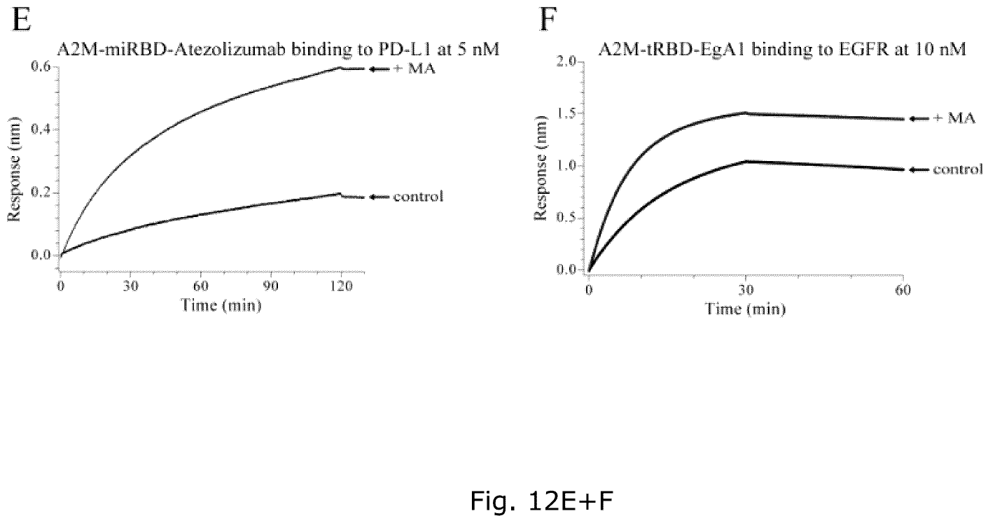

To determine whether shielding of biopharmaceutical moieties in A2M could be accomplished by their incorporation into A2M in other ways than the ciRBD approach used previously, four new fusion approaches were tested. In the first, the EgA1 nanobody was expressed immediately following the C-terminus of A2M's RBD domain to produce the A2M-fusion-EgA1 protein. A2M-fusion-EgA1 did not demonstrate conformational dependence of EgA1's binding to EGFR (FIG. 12A), indicating that not all positions adjacent to the RBD domain are shielded in the native conformation of A2M.

In the second approach, iRBD, the EgA1 nanobody was inserted into the RBD domain replacing A2M residues 1392-1403. The resulting A2M-iRBD-EgA1 protein showed a high degree of conformational dependence (FIG. 12B), comparable to that of the ciRBD approach (see Example 3). This indicates that the region of A2M's RBD domain adjacent to residues 1393-1403 is an appropriate site for the incorporation of biopharmaceutical moieties to achieve conformational dependence. Accordingly, the third approach, miRBD, also achieved conformational dependence by incorporation of either the EgA1 nanobody, the KN035 nanobody, or the Atezolizumab scFv at a position replacing A2M residues 1393-1395 (FIG. 12B-E).

In the fourth approach, tRBD, coiled-coil interactions were used to bring an incorporated biopharmaceutical moiety (EgA1) into proximity of the RBD residues 1393-1403. The EgA1 nanobody was incorporated at a position C-terminal to the RBD domain with an alpha-helix sequence designed to be complementary to A2M residues 1393-1403 at its N-terminus. The alpha-helix sequence is attached to the RBD domain with a 15-residue linker, in order to permit the alpha-helix to interact with residues 1393-1403. Furthermore, modifications to A2M residues 1393-1403 were made to enhance the designed complementary coiled-coil interactions. The resulting A2M-tRBD-EgA1 protein demonstrated conformational dependence of the EgA1/EGFR interaction (FIG. 12F), indicating that the coiled-coil interactions were able to bring the EgA1 nanobody into proximity with residues 1393-1403 and that this proximity conveyed at least partial shielding of the EgA1 nanobody in the native conformation of A2M.

### Conclusion

Investigations of the fusion, iRBD, miRBD, and tRBD approaches to producing fusion constructs of A2M and biopharmaceutical moieties showed that positions that are proximal to A2M RBD residues 1393-1403 due to either direct fusion at this site (as seen in the ciRBD, iRBD, and ciRBD approaches) or localization of the moiety to this position through other means (e.g. through coiled-coil interactions, as demonstrated by A2M-tRBD-EgA1) convey conformationally dependent target binding for many different tested biopharmaceutical moieties (16 in total, considering all ciRBD, iRBD, miRBD, and tRBD fusion constructs).

### Example 11—Study of Insertion Sites in the RBD Region

### Aim

To identify sites for drug insertion in the RBD of A2M which will convey conformation-dependent accessibility.

### Materials and Methods

Figures were prepared using the PyMol Molecular Graphics System software (version 2.3.0).

### Results

In the iRBD, miRBD, and ciRBD approaches to creating A2M-based prodrugs, residues 1392 to 1403 of A2M's RBD domain are either replaced with the drug sequence (as well as N- and C-terminal linkers) or the drug is inserted between residues 1402-1403 without altering any residues of A2M. Residues 1391-1405 or 1392-1404 comprise a loop or linker region between strands of beta-sheet structure, and such loops are well-suited for modification, in contrast to the beta-sheet sequence where modifications are more likely to affect the folding of the domain.

Furthermore, the orientation of the loop is also critical to achieving a conformationally dependent drug position, as fusion of a drug at the opposite side of the RBD at position 1474 (in the A2M-fusion-EgA1 construct) produced an always-accessible drug.

After a structural evaluation of the RBD domain, three additional loop regions were identified that are suitable for drug insertion, namely the regions comprising residues 1368-1379 (loop 1), 1420-1426 (loop 3), and 1450-1457 (loop 4), in addition to the empirically tested region comprising residues 1392-1404 (loop 2) (FIG. 13). All three loops extend between beta-strands and are oriented in a similar direction to loop 2 (1392-1404), where they face inwards towards the interior of the A2M tetramer.

### Conclusion

In addition to the region comprising residues 1392-1404 (loop 2), three additional loops comprising residues 1368-1379 (loop 1), 1420-1426 (loop 3), and 1450-1457 (loop 4) of A2M were identified that are considered usable for replacement by or direct insertion of one or more drugs, in order to convey conformational-dependent binding of their therapeutic target.

### SEQUENCE LISTING

The present specification makes reference to a Sequence Listing (submitted electronically as an XML file named “SEQUENCE LIST 77582PC01 26 01 23” on 31 Jan. 2023). The XML file was generated on 26 Jan. 2023 and is 426 KB in size. The entire contents of the sequence listing are herein incorporated by reference.

The sequence listing contains the following sequences:
SEQ ID NO: 1—Recombinant wildtype A2M—Protein sequence
SEQ ID NO: 2—Recombinant wildtype A2M—DNA sequence
SEQ ID NO: 3—RBD domain (aa 1335-1474 of wildtype A2M)—Protein sequence
SEQ ID NO: 4—Bait region (aa 690-728 of wildtype A2M)—Protein sequence
SEQ ID NO: 5—A2M ciRBD EgA1—Protein sequence
SEQ ID NO: 6—A2M ciRBD EgA1—DNA sequence
SEQ ID NO: 7—A2M ciRBD Atezolizumab K1393A K1397A—Protein sequence
SEQ ID NO: 8—A2M ciRBD Atezolizumab K1393A K1397A—DNA sequence
SEQ ID NO: 9—A2M ciRBD KN035 K1393A K1397A—Protein sequence
SEQ ID NO: 10—A2M ciRBD KN035 K1393A K1397A—DNA sequence
SEQ ID NO: 11—A2M ciRBD Nivolumab—Protein sequence
SEQ ID NO: 12—A2M ciRBD Nivolumab—DNA sequence
SEQ ID NO: 13—A2M ciRBD Ipilimumab—Protein sequence
SEQ ID NO: 14—A2M ciRBD Ipilimumab—DNA sequence
SEQ ID NO: 15—A2M ciRBD Foralumab—Protein sequence
SEQ ID NO: 16—A2M ciRBD Foralumab—DNA sequence
SEQ ID NO: 17—A2M ciRBD Muromonab—Protein sequence
SEQ ID NO: 18—A2M ciRBD Muromonab—DNA sequence
SEQ ID NO: 19—A2M ciRBD Urelumab—Protein sequence
SEQ ID NO: 20—A2M ciRBD Urelumab—DNA sequence
SEQ ID NO: 21—A2M ciRBD Adalimumab—Protein sequence
SEQ ID NO: 22—A2M ciRBD Adalimumab—DNA sequence
SEQ ID NO: 23—A2M ciRBD IL2—Protein sequence
SEQ ID NO: 24—A2M ciRBD IL2—DNA sequence
SEQ ID NO: 25—A2M ciRBD PD1—Protein sequence
SEQ ID NO: 26—A2M ciRBD PD1—DNA sequence
SEQ ID NO: 27—EgA1 nanobody—Protein sequence
SEQ ID NO: 28—Atezolizumab scFv (VH_VL)—Protein sequence
SEQ ID NO: 29—KN035 nanobody—Protein sequence
SEQ ID NO: 30—Nivolumab scFv (VH_VL)—Protein sequence
SEQ ID NO: 31—Ipilimumab scFv (VH_VL)—Protein sequence
SEQ ID NO: 32—Foralumab scFv (VH_VL)—Protein sequence
SEQ ID NO: 33—Muromonab scFv (VH_VL)—Protein sequence
SEQ ID NO: 34—Urelumab scFv (VH_VL)—Protein sequence
SEQ ID NO: 35—Adalimumab scFv (VH_VL)—Protein sequence
SEQ ID NO: 36—IL2 cytokine—Protein sequence
SEQ ID NO: 37—EGFR extracellular region—Protein sequence
SEQ ID NO: 38—PDL-1 extracellular region—Protein sequence
SEQ ID NO: 39—PD-1 extracellular region, C93S mutation—Protein sequence
SEQ ID NO: 40—CTLA-4 extracellular region—Protein sequence
SEQ ID NO: 41—CD3γε extracellular region—Protein sequence
SEQ ID NO: 42—4—1BB extracellular region—Protein sequence
SEQ ID NO: 43—IL-2Rα extracellular region, C213A mutation—Protein sequence
SEQ ID NO: 44—Human IgG1 Fc region—Protein sequence
SEQ ID NO: 45—EGFR extracellular region—Protein sequence
SEQ ID NO: 46—EGFR extracellular region-DNA sequence
SEQ ID NO: 47—PDL-1 extracellular region—Protein sequence
SEQ ID NO: 48—PDL-1 extracellular region—DNA sequence
SEQ ID NO: 49—PD-1 extracellular region, C93S mutation—Protein sequence
SEQ ID NO: 50—PD-1 extracellular region, C93S mutation—DNA sequence
SEQ ID NO: 51—CTLA-4 extracellular region—Protein sequence
SEQ ID NO: 52—CTLA-4 extracellular region—DNA sequence
SEQ ID NO: 53—CD3γε extracellular region—Protein sequence
SEQ ID NO: 54—CD3γε extracellular region—DNA sequence
SEQ ID NO: 55—4—1BB extracellular region—Protein sequence
SEQ ID NO: 56—4—1BB extracellular region—DNA sequence
SEQ ID NO: 57—IL-2Rα extracellular region, C213A—Protein sequence
SEQ ID NO: 58—IL-2Rα extracellular region, C213A—DNA sequence
SEQ ID NO: 59—TNFα cytokine, StrepII tags and A2M leader peptide—Protein sequence
SEQ ID NO: 60—TNFα cytokine, StrepII tags and A2M leader peptide—DNA sequence
SEQ ID NO: 61—pro MMP2, with N-terminal Strep tags—Protein sequence
SEQ ID NO: 62—pro MMP2, with N-terminal Strep tags—DNA sequence
SEQ ID NO: 63—LRP1 cluster 1B with Nt Strep tags and Ct hFc—Protein sequence
SEQ ID NO: 64—LRP1 cluster 1B with Nt Strep tags and Ct hFc—DNA sequence
SEQ ID NO: 65—A2M with modified bait region, TR—Protein sequence
SEQ ID NO: 66—A2M with modified bait region, TR K704—Protein sequence
SEQ ID NO: 67—A2M with modified bait region, TR A21A—Protein sequence
SEQ ID NO: 68—A2M with modified bait region, TR B74—Protein sequence
SEQ ID NO: 69—A2M with modified bait region, TR C9—Protein sequence
SEQ ID NO: 70—A2M with modified bait region, TR S1—Protein sequence
SEQ ID NO: 71—A2M with modified bait region, TRΔ7 S1 I710—Protein sequence
SEQ ID NO: 72—A2M with modified bait region, TR S1 QRT4—Protein sequence
SEQ ID NO: 73—A2M with modified bait region, TRΔ7 S1 I703—Protein sequence
SEQ ID NO: 74—A2M ciRBD Atez, K1393A K1397A T654C T661C, bait region TRΔ7 S1 I703—Protein sequence
SEQ ID NO: 75—A2M ciRBD Atez, K1393A K1397A T654C T661C, bait region TRΔ7 S1 I703—DNA sequence
SEQ ID NO: 76—A2M ciRBD Atez, K1393A K1397A T654C T661C, bait region TRΔ7 S1 I703 P704—Protein sequence
SEQ ID NO: 77—A2M ciRBD Atez, K1393A K1397A T654C T661C, bait region TRΔ7 S1 I703 P704—DNA sequence
SEQ ID NO: 78—The N-terminal linker of the ciRBD format—Protein sequence
SEQ ID NO: 79—The C-terminal linker of the ciRBD format—Protein sequence
SEQ ID NO: 80—The N-terminal linker of the iRBD format—Protein sequence
SEQ ID NO: 81—The C-terminal linker of the iRBD format—Protein sequence
SEQ ID NO: 82—One tested N-terminal linker of the miRBD format—Protein sequence
SEQ ID NO: 83—One tested C-terminal linker of the miRBD format—Protein sequence
SEQ ID NO: 84—A2M iRBD N15/C12 EgA1—Protein sequence
SEQ ID NO: 85—A2M iRBD N15/C12 EgA1—DNA sequence
SEQ ID NO: 86—A2M miRBD N18/C15 EgA1—Protein sequence
SEQ ID NO: 87—A2M miRBD N18/C15 EgA1—DNA sequence
SEQ ID NO: 88—A2M miRBD N18/C15 Atezolizumab—Protein sequence
SEQ ID NO: 89—A2M miRBD N18/C15 Atezolizumab—DNA sequence
SEQ ID NO: 90—A2M miRBD N18/C15 KN035—Protein sequence
SEQ ID NO: 91—A2M miRBD N18/C15 KN035—DNA sequence
SEQ ID NO: 92—A2M tRBD15b EgA1—Protein sequence
SEQ ID NO: 93—A2M tRBD15b EgA1—DNA sequence
SEQ ID NO: 94—A2M with EgA1 nanobody immediately after RBD C-terminus—Protein sequence
SEQ ID NO: 95—A2M with EgA1 nanobody immediately after RBD C-terminus—DNA sequence
SEQ ID NO: 96—Cleavage site for MMP (A21A)—Protein sequence
SEQ ID NO: 97—Cleavage site for MMP (B74)—Protein sequence
SEQ ID NO: 98—Cleavage site for MMP (C9)—Protein sequence
SEQ ID NO: 99—Cleavage site for MMP (S1)—Protein sequence
SEQ ID NO: 100—Cleavage site for MMP (S1P)—Protein sequence
SEQ ID NO: 101—MMP cleavage site and R from wildtype bait region—Protein sequence
SEQ ID NO: 102—Cleavage site—Protein sequence
SEQ ID NO: 103—Cleavage site—Protein sequence
SEQ ID NO: 104—Cleavage site—Protein sequence
SEQ ID NO: 105—Cleavage site—Protein sequence
SEQ ID NO: 106—Cleavage site—Protein sequence
SEQ ID NO: 107—Cleavage site—Protein sequence
SEQ ID NO: 108—Cleavage site—Protein sequence
SEQ ID NO: 109—Cleavage site—Protein sequence
SEQ ID NO: 110—Cleavage site—Protein sequence
SEQ ID NO: 111—Cleavage site—Protein sequence
SEQ ID NO: 112—Cleavage site—Protein sequence
SEQ ID NO: 113—Cleavage site—Protein sequence
SEQ ID NO: 114—Cleavage site—Protein sequence
SEQ ID NO: 115—Cleavage site—Protein sequence
SEQ ID NO: 116—Cleavage site—Protein sequence
SEQ ID NO: 117—Cleavage site—Protein sequence
SEQ ID NO: 118—Cleavage site—Protein sequence
SEQ ID NO: 119—Cleavage site—Protein sequence
SEQ ID NO: 120—Cleavage site—Protein sequence
SEQ ID NO: 121—Cleavage site—Protein sequence
SEQ ID NO: 122—Cleavage site—Protein sequence
SEQ ID NO: 123—Cleavage site—Protein sequence
SEQ ID NO: 124—Tabula rasa region (TR)—Protein sequence
SEQ ID NO: 125—TR K704—Protein sequence
SEQ ID NO: 126—TR A21A—Protein sequence
SEQ ID NO: 127—TR B74—Protein sequence
SEQ ID NO: 128—TR C9—Protein sequence
SEQ ID NO: 129—TRS1—Protein sequence
SEQ ID NO: 130—TR S1 QRT4—Protein sequence
SEQ ID NO: 131—TRΔ7 S1 I710—Protein sequence
SEQ ID NO: 132—TRΔ7 S1 I703—Protein sequence
SEQ ID NO: 133—TRΔ7 S1 I703 P704—Protein sequence
SEQ ID NO: 134—CPAMD1 (a.k.a. C3)—NP_000055.2—Protein sequence
SEQ ID NO: 135—CPAMD2 (a.k.a. C4A)—NP_009224.2—Protein sequence
SEQ ID NO: 136—CPAMD3 (a.k.a. C4B)—NP_001002029.3—Protein sequence
SEQ ID NO: 137—CPAMD4 (a.k.a. C5)—NP_001726.2—Protein sequence
SEQ ID NO: 138—CPAMD5 (a.k.a. A2M)—NP_000005.3—Protein sequence
SEQ ID NO: 139—CPAMD6 (a.k.a. PZP)—NP_002855.2—Protein sequence
SEQ ID NO: 140—CPAMD7 (a.k.a. CD109)—NP_598000.2—Protein sequence
SEQ ID NO: 141—CPAMD8—NP_056507.3—Protein sequence
SEQ ID NO: 142—CPAMD9 (a.k.a. A2ML1)—NP_653271.3—Protein sequence
SEQ ID NO: 143—Ovostatin 1—Q6IE37.2—Protein sequence
SEQ ID NO: 144—Ovostatin 2—Q6IE36.2—Protein sequence
SEQ ID NO: 145—188—see table 1
SEQ ID NO: 189—210—see table 2
SEQ ID NO: 211—222—see table 3
SEQ ID NO: 223—GS linker

1. A proteinaceous prodrug construct, comprising:
(a) a complement 3- and pregnancy zone protein-like, alpha-2-macroglobulin domain-containing (CPAMD) protein or a fragment thereof, and
(b) one or more drugs,
wherein:
(i) the CPAMD protein or fragment thereof comprises (1) a bait region with at least one protease cleavage site, and (2) a Receptor Binding Domain (RBD),
(ii) the one or more drugs are positioned inside or in the vicinity of the RBD, and
(iii) the CPAMD protein or fragment thereof shields the one or more drugs and is capable of altering conformation upon proteolytic cleavage of the at least one protease cleavage site, making the one or more drugs accessible.

2. The proteinaceous prodrug construct according to claim 1, wherein the one or more drugs is positioned inside or in the vicinity of any one of loops 1-4 of the RBD.

3. The proteinaceous prodrug construct according to claim 1, wherein the proteinaceous prodrug construct is a fusion protein.

4. The proteinaceous prodrug construct according to claim 2, wherein the one or more drugs are positioned inside any one of loops 1-4 of the RBD, and optionally wherein the loop is modified, in relation to a wildtype loop sequence, by addition, substitution or deletion of one or more amino acids to accommodate the one or more drugs.

5. The proteinaceous prodrug construct according to claim 4, wherein the one or more drugs replace one or more amino acids of the loop.

6. The proteinaceous prodrug construct according claim 2, wherein the one or more drugs is positioned in the vicinity of any one of loops 1-4 of the RBD, and wherein the proteinaceous prodrug construct comprises a first interaction domain and the one or more drugs comprise a second interaction domain, wherein the first and second interaction domains form a complex positioning the one or more drugs in the vicinity of the loop.

7. The proteinaceous prodrug construct according to claim 1, wherein the proteinaceous prodrug construct is capable of forming a multimer.

8. The proteinaceous prodrug construct according to claim 1, wherein the CPAMD protein is selected from A2M, PZP, Ovostatin 1, Ovostatin 2, CPAMD1, CPAMD2, CPAMD3, CPAMD4, CPAMD7, CPAMD8, CPAMD9, and functional homolog thereof.

9. The proteinaceous prodrug construct according to claim 8, wherein the CPAMD protein is human A2M, or a functional homolog thereof.

10. The proteinaceous prodrug construct according to claim 9, wherein:
(i) the functional homolog of human A2M has an amino acid sequence that is at least about 80% identical to the amino acid sequence set forth in SEQ ID NO: 1;
(ii) the human A2M has an amino acid sequence that is at least about 95% identical to, or identical to, the amino acid sequence set forth in SEQ ID NO: 1.

11. The proteinaceous prodrug construct according to claim 9, wherein the one or more drugs are positioned within a region comprising amino acid 1368-1379, 1392-1404, 1420-1426, or 1450-1457 of human A2M.

12. The proteinaceous prodrug construct according to claim 1, wherein the one or more drugs are selected from the group consisting of: an antigen-targeting moiety, a receptor ligand, the extracellular region of a cell surface receptor, the extracellular region of a cell surface ligand, and a receptor agonist.

13. The proteinaceous prodrug construct according to claim 1, wherein the bait region is modified to be selectively cleaved by one or more proteases.

14. The proteinaceous prodrug construct according to claim 1, wherein the bait region has been modified to be free from protease cleavage sites recognized by human proteases except for a single cleavage site.

15. The proteinaceous prodrug construct according to claim 1, wherein the one or more drugs is an antibody, or antigen-binding fragment thereof, that specifically binds to antigen selected from the group consisting of IL-2, EGFR, PDL-1, PD-1, CTLA-4, CD3γε, 4—1BB, IL-2Rα, and TNFα.

16. The proteinaceous prodrug construct according to claim 15, wherein the one or more drugs are selected from the group consisting of Atezolizumab, EgA1, Ipilimumab, Nivolumab, KN035, Urelumab, Foralumab, Muromonab, Adalimumab, and therapeutically active antigen-binding fragments or variants of each.

17. The proteinaceous prodrug construct according to claim 1, wherein the one or more drugs is a cytokine, or a therapeutically active fragment or variant thereof, selected from the group consisting of IL1, IL1alpha, IL1beta, IL2, IL3, IL4, IL6, IL7, IL8, IL9, IL10, IL11, IL12, IL13, IL14, IL15, IL16, IL17, 118, IL19, IL20, IL21, IL22, IL23, IL24, IL25, IL26, IL27, IL28, IL29, IL30, IL31, IL32, IL33, IL34, IL35, IL36, GM-CSF, TGF-β, CSF-1, insulin, GLP-1, HGH, VEGF, PDGF, BMP, EPO, G-CSF, IL-11, IFN-α, IFN-β and IFN-γ.

18. The proteinaceous prodrug construct according to claim 1, wherein the proteinaceous prodrug construct is encoded by an amino acid sequence selected from the group consisting of: SEQ ID NO: 5, SEQ ID NO:7, SEQ ID NO:9, SEQ ID NO:11, SEQ ID NO: 13, SEQ ID NO:15, and SEQ ID NO:17, SEQ ID NO:19, SEQ ID NO:21, SEQ ID NO: 23, or SEQ ID NO:25.

19. A nucleic acid encoding a proteinaceous prodrug construct according to claim 1.

20. A vector comprising the nucleic acid according to claim 19.

21. A host cell comprising a vector according to claim 20.

22. A method of treating or preventing a disease or disorder in a subject in need thereof, wherein the method comprises administering a therapeutically effective amount of the proteinaceous prodrug construct according to claim 1 to the subject.

23. A method of treating or preventing a disease or disorder in a subject in need thereof, wherein the method comprises administering a therapeutically effective amount of the nucleic acid according to claim 19 to the subject.

24. A method of treating or preventing a disease or disorder in a subject in need thereof, wherein the method comprises administering a therapeutically effective amount of the host cell of claim 21 to the subject.

25. The method of claim 22, wherein the disease or disorder is a neoplasm, a blood disorder, a metabolic disorder, an autoimmune disease, an immunodeficiency, or an infectious disease.

26. A method for producing the proteinaceous prodrug construct according to claim 1, the method comprising:
(i) introducing into a host cell an expression vector comprising a nucleic acid encoding the proteinaceous prodrug construct;
(ii) growing the host cell under conditions that allow for expression of the proteinaceous prodrug construct from the vector; and
(iii) purifying the proteinaceous prodrug construct.
